SEQUENCE LISTING (60 sequences)

SEQ ID NO: 1 - protein, 574 aa, Homo sapiens
MCPRAARAPATLLLALGAVLWPAAGAWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHTDEMFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGRIKFSTGSHCHGSFSLIFLSLWAVIFVLYQ

SEQ ID NO: 2 - protein, 5 aa, Artificial Sequence
  heavy complementarity-determining region
SYNMY

SEQ ID NO: 3 - protein, 17 aa, Artificial Sequence
  heavy complementarity-determining region
  MISC_FEATURE (13)..(13): X = Gln or Leu
  MISC_FEATURE (14)..(14): X = Lys or Thr
YIDPYNGGSSYNXXFKG

SEQ ID NO: 4 - protein, 11 aa, Artificial Sequence
  heavy complementarity-determining region
  MISC_FEATURE (3)..(3): X = Asp or Gly
GYXNYKAWFAY

SEQ ID NO: 5 - protein, 11 aa, Artificial Sequence
  light complementarity-determining region
  MISC_FEATURE (7)..(7): X = Thr or Ser
KASQSVXNDVA

SEQ ID NO: 6 - protein, 7 aa, Artificial Sequence
  light complementarity-determining region
  MISC_FEATURE (4)..(4): X = Thr or Asn
YASXRYT

SEQ ID NO: 7 - protein, 8 aa, Artificial Sequence
  light complementarity-determining region
QQDYSSLT

SEQ ID NO: 8 - protein, 17 aa, Artificial Sequence
  heavy complementarity-determining region
YIDPYNGGSSYNLTFKG

SEQ ID NO: 9 - protein, 11 aa, Artificial Sequence
  heavy complementarity-determining region
GYGNYKAWFAY

SEQ ID NO: 10 - protein, 11 aa, Artificial Sequence
  light complementarity-determining region
KASQSVSNDVA

SEQ ID NO: 11 - protein, 7 aa, Artificial Sequence
  light complementarity-determining region
YASTRYT

SEQ ID NO: 12 - protein, 17 aa, Artificial Sequence
  heavy complementarity-determining region
YIDPYNGGSSYNQKFKG

SEQ ID NO: 13 - protein, 11 aa, Artificial Sequence
  heavy complementarity-determining region
GYNNYKAWFAY

SEQ ID NO: 14 - protein, 11 aa, Artificial Sequence
  light complementarity-determining region
KASQSVTNDVA

SEQ ID NO: 15 - protein, 7 aa, Artificial Sequence
  light complementarity-determining region
YASNRYT

SEQ ID NO: 16 - protein, 330 aa, Artificial Sequence
  heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 330 aa, Artificial Sequence
  heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 330 aa, Artificial Sequence
  heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 330 aa, Artificial Sequence
  heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - DNA, 45 bp, Artificial Sequence
  primer
tacgactcacaagcttgccgccaccatgtgtccccgagccgcgcg

SEQ ID NO: 21 - DNA, 57 bp, Artificial Sequence
  primer
ccgccccgactctagatcagtgatggtgatgatggtgcttgatccgaccttcaactg

SEQ ID NO: 22 - protein, 553 aa, Homo sapiens
MCPRAARAPATLLLALGAVLWPAAGAWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHTDEMFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGRIKHHHHHH

SEQ ID NO: 23 - protein, 120 aa, Artificial Sequence [sequence omitted]

SEQ ID NO: 24 - protein, 107 aa, Artificial Sequence [sequence omitted]

SEQ ID NO: 25 - protein, 450 aa, Artificial Sequence
  heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 214 aa, Artificial Sequence
  light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 450 aa, Artificial Sequence
  heavy chain
QVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 28 - protein, 214 aa, Artificial Sequence
  light chain
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 120 aa, Artificial Sequence
  heavy chain variant
QIQLVQSGAEVKKPGASVKVSCKASGYTFTSYNMYWVRQAPGQRLEWMGYIDPYNGGSSYNLTFKGRVTITRDKSASTAYMELSSLRSEDTAVYYCARGYGNYKAWFAYWGQGTLVTVSS

SEQ ID NO: 30 - protein, 120 aa, Artificial Sequence
  heavy chain variant
QIQLVQSGAEVKKPGASVKVSCKASGYAFTSYNMYWVRQAPGQSLEWMGYIDPYNGGSSYNLTFKGRVTITVDKSASTAYMELSSLRSEDTAVYYCARGYGNYKAWFAYWGQGTLVTVSS

SEQ ID NO: 31 - protein, 120 aa, Artificial Sequence
  heavy chain variant
QIQLVQSGAEVKKPGASVKVSCKASGYAFTSYNMYWVRQAPGQSLEWIGYIDPYNGGSSYNLTFKGKATITVDKSASTAYMELSSLRSEDTAVYYCARGYGNYKAWFAYWGQGTLVTVSS

SEQ ID NO: 32 - protein, 120 aa, Artificial Sequence
  heavy chain variant
QIQLVQSGAEVKKPGASVKVSCKASGYAFASYNMYWVRQAPGQSLEWIGYIDPYNGGSSYNLTFKGKATLTVDKSASTAYMELSSLRSEDTAVYYCARGYGNYKAWFAYWGQGTLVTVSS

SEQ ID NO: 33 - protein, 106 aa, Artificial Sequence
  light chain variant
DIQMTQSPSSLSASVGDRVTITCKASQSVSNDVAWYQQKPGKAPKLLIYYASTRYTGVPSRFSGSGYGTDFTFTISSLQPEDIATYYCQQDYSSLTFGQGTKLEIK

SEQ ID NO: 34 - protein, 106 aa, Artificial Sequence
  light chain variant
DIQMTQSPSSLSASVGDRVTITCKASQSVSNDVAWYQQKPGKAPKLLIYYASTRYTGVPDRFSGSGYGTDFTFTISSLQPEDIATYYCQQDYSSLTFGQGTKLEIK

SEQ ID NO: 35 - protein, 106 aa, Artificial Sequence
  light chain variant
DVQMTQSPSSLSASVGDRVTITCKASQSVSNDVAWYQQKPGKAPKLLIYYASTRYTGVPDRFSGSGYGTDFTFTISSLQPEDIATYYCQQDYSSLTFGQGTKLEIK

SEQ ID NO: 36 - protein, 106 aa, Artificial Sequence
  light chain variant
DVQMTQSPSSLSASVGDRVTITCKASQSVSNDVAWYQQKPGKAPKLLIYYASTRYTGVPDRFSGSGYGTDFTFTISSLQPEDIATYFCQQDYSSLTFGQGTKLEIK

SEQ ID NO: 37 - protein, 120 aa, Artificial Sequence
  heavy chain variant
QIQLVQSGAEVKKPGASVKVSCKASGYTFASYNMYWVRQAPGQRLEWIGYIDPYNGGSSYNLTFKGRVTLTRDKSASTAYMELSSLRSEDTAVYYCARGYGNYKAWFAYWGQGTLVTVSS

SEQ ID NO: 38 - protein, 450 aa, Artificial Sequence
  heavy chain
QIQLVQSGAEVKKPGASVKVSCKASGYTFASYNMYWVRQAPGQRLEWIGYIDPYNGGSSYNLTFKGRVTLTRDKSASTAYMELSSLRSEDTAVYYCARGYGNYKAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 213 aa, Artificial Sequence
  light chain
DIQMTQSPSSLSASVGDRVTITCKASQSVSNDVAWYQQKPGKAPKLLIYYASTRYTGVPSRFSGSGYGTDFTFTISSLQPEDIATYYCQQDYSSLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 120 aa, Artificial Sequence
  heavy chain
EIQLQQSGPELVKPGASVKVSCKASGYAFTSYNMYWVKQSHGKRLEWIGYIDPYNGGSSYNQKFKGKATLTVDKSSSTAYMHLNNLTSEDSAVYYCARGYNNYKAWFAYWGQGTLVTVSA

SEQ ID NO: 41 - protein, 106 aa, Artificial Sequence
  light chain
DVVMTQTPKFLLVSAGDRVTITCKASQSVTNDVAWYQQKPGQSPKLLIYYASNRYTGVPDRFTGSGYGTDFTFTISTMQAEDLAVYFCQQDYSSLTFGAGTKLELK

SEQ ID NO: 42 - protein, 120 aa, Artificial Sequence
  heavy chain
QIQLVQSGAEVKKPGASVKVSCKASGYTFASYNMYWVRQAPGQRLEWIGYIDPYNGGSSYNQKFKGRVTLTRDKSASTAYMELSSLRSEDTAVYYCARGYNNYKAWFAYWGQGTLVTVSS

SEQ ID NO: 43 - protein, 106 aa, Artificial Sequence
  light chain
DIQMTQSPSSLSASVGDRVTITCKASQSVTNDVAWYQQKPGKAPKLLIYYASNRYTGVPSRFSGSGYGTDFTFTISSLQPEDIATYYCQQDYSSLTFGQGTKLEIK

SEQ ID NO: 44 - protein, 106 aa, Artificial Sequence
  light chain
DIQMTQSPSSLSASVGDRVTITCKASQSVTNDVAWYQQKPGKAPKLLIYYASNRYTGVPDRFSGSGYGTDFTFTISSLQPEDIATYYCQQDYSSLTFGQGTKLEIK

SEQ ID NO: 45 - protein, 106 aa, Artificial Sequence
  light chain
DVQMTQSPSSLSASVGDRVTITCKASQSVTNDVAWYQQKPGKAPKLLIYYASNRYTGVPDRFSGSGYGTDFTFTISSLQPEDIATYYCQQDYSSLTFGQGTKLEIK

SEQ ID NO: 46 - protein, 106 aa, Artificial Sequence
  light chain
DVQMTQSPSSLSASVGDRVTITCKASQSVTNDVAWYQQKPGKAPKLLIYYASNRYTGVPDRFSGSGYGTDFTFTISSLQPEDIATYFCQQDYSSLTFGQGTKLEIK

SEQ ID NO: 47 - protein, 450 aa, Artificial Sequence
  heavy chain
QIQLVQSGAEVKKPGASVKVSCKASGYTFASYNMYWVRQAPGQRLEWIGYIDPYNGGSSYNQKFKGRVTLTRDKSASTAYMELSSLRSEDTAVYYCARGYNNYKAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 213 aa, Artificial Sequence
  light chain
DIQMTQSPSSLSASVGDRVTITCKASQSVTNDVAWYQQKPGKAPKLLIYYASNRYTGVPSRFSGSGYGTDFTFTISSLQPEDIATYYCQQDYSSLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 120 aa, Artificial Sequence
  variable heavy complementarity-determining region
EFQLQQSGPELVKPGASVKVSCKASGYAFTSYNMYWVKQSHGKRLEWIGYIDPYNGGSSYNQKFKGKATLTVDKSSSTAYMHLNNLTSEDSAVYYCARGYNNYKAWFAYWGQGTLVTVSA

SEQ ID NO: 50 - protein, 106 aa, Artificial Sequence
  variable light complementarity-determining region
SIVMTQTPKFLLVSAGDRVTITCKASQSVTNDVAWYQQKPGQSPKLLIYYASNRYTGVPDRFTGSGYGTDFTFTISTMQAEDLAVYFCQQDYSSLTFGAGTKLELK

SEQ ID NO: 51 - protein, 120 aa, Artificial Sequence
  variable heavy complementarity-determining region
EIQLQQSGPELVKPGASVKVSCKASGYAFTSYNMYWVKQSHGKSLEWIGYIDPYNGGTSYNQKFKGKATLTVDKSSSTAYMHLNSLTSEDSAVYYCARGYGNYKAWFAYWGQGTLVTVSA

SEQ ID NO: 52 - protein, 106 aa, Artificial Sequence
  variable light complementarity-determining region
DAVMTQTPKFLLVSAGDRVTITCKASQSVTNDVAWYQQKPGQSPKLLIYYASNRYTGVPDRFTGSGYGTDFTFTISTVQAEDLAVYFCQQDYSSLTFGAGTKLELK

SEQ ID NO: 53 - protein, 126 aa, Artificial Sequence
  variable heavy complementarity-determining region
EVQLQQSGPELVKPGASVKVSCKASGYAFASYNMNWVKQSHGKSLDWIGYIDPYNGGSSYNLTFKGKATLTVDKSSTTAYMHLNSLTSEDSAVYYCARGYGNYKAWFAYWGQGTLVTVSAASTKGP

SEQ ID NO: 54 - protein, 112 aa, Artificial Sequence
  variable light complementarity-determining region
SIVMTPTPKFLLVSAGDRVTITCKASQSVSNDVAWYQQKPGQSPKLLIYYASTRYTGVPDRFTGSGYGTDFTFTISTVQAEDLAVYFCQQDYSSLTFGAGTKLELKRTVAAP

SEQ ID NO: 55 - protein, 126 aa, Artificial Sequence
  variable heavy complementarity-determining region
QIQLQQSGPELVKPGASVKVSCKASGYAFASYNMNWVKQSHGKSLDWIGYIDPYNGGSSYNLTFKGKATLTVDKSSTTAYMHLNSLTSEDSAVYYCARGYGNYKAWFAYWGQGTLVTVSAASTKGP

SEQ ID NO: 56 - protein, 112 aa, Artificial Sequence
  variable light complementarity-determining region
DVVMTQTPKFLLVSAGDRVTITCKASQSVSNDVAWYQQKPGQSPKLLIYYASTRYTGVPDRFTGSGYGTDFTFTISTVQAEDLAVYFCQQDYSSLTFGAGTKLELKRTVAAP

SEQ ID NO: 57 - protein, 124 aa, Artificial Sequence
  variable heavy complementarity-determining region
EVQLLESGGGLVQPGGSLRLSCAASGFTFHRYAMSWVRQAPGKGLEWVSAISGSGMNTYYADSVKGRFTISRDNSKNTLYLQMNSLRAEDTAVYYCARGGLYGSGSYLSDFDLWGRGTLVTVSS

SEQ ID NO: 58 - protein, 107 aa, Artificial Sequence
  variable light complementarity-determining region
EIVLTQSPATLSVSPGERATLSCRASQSVGSNLAWYQQKPGQAPRLLIYGASTRATGIPARFSGSGSGTEFTLTISSLQSEDFAVYYCQQHNAFPYTFGGGTKVEIK

SEQ ID NO: 59 - protein, 120 aa, Artificial Sequence
  variable heavy complementarity-determining region
EVQLQESGPGLVKPSETLSLTCAVSGYSITSGYYWNWIRQPPGKKLEWMGYINYGGSNGYNPSLKSRITISRDTSKNQFSLKLSSVTAADTAVYYCARDYDAYYEALDDWGQGTTVTVSS

SEQ ID NO: 60 - protein, 106 aa, Artificial Sequence
  variable light complementarity-determining region
EIVLSQSPATLSLSPGERATLSCRASSRVNYMHWYQQKPGQSPRPWISATSNLASGVPARFSGSGSGTSYTLTISSLEPEDFAVYYCQQWSSNPPTFGGGTKVEIK

(19) United States
(12) Patent Application Publication
(10) Pub. No.: US 2024/0270865 A1
(43) Pub. Date: Aug. 15, 2024

(54) CANCER TREATMENT METHODS USING ANTI-CD73 ANTIBODIES

(71) Applicant: INNATE PHARMA, MARSEILLE (FR)

(72) Inventors: LAURENT GAUTHIER, MARSEILLE (FR); CARINE PATUREL, MARCY L'ETOILE (FR); IVAN PERROT, MARSEILLE (FR)

(21) Appl. No.: 18/020,955

(22) PCT Filed: Aug. 6, 2021
(86) PCT No.: PCT/EP2021/072020

§ 371 Date: Feb. 13, 2023

Related U.S. Application Data
(60) Provisional application No. 63/065,085, filed on Aug. 13, 2020.

(51) Int. Cl. C07K 16/28; A61K 31/337; A61K 39/00; A61P 35/00; C07K 16/32

(52) U.S. Cl. CPC C07K 16/2896; A61K 31/337; A61P 35/00; C07K 16/32; A61K 2039/507; A61K 2039/545; C07K 2317/24; C07K 2317/33; C07K 2317/56; C07K 2317/76

(57) ABSTRACT

The disclosure relates to methods and compositions for the treatment of cancer. Specifically, the disclosure relates to methods for administering anti-CD73 antibodies, and to methods for using anti-CD73 antibodies for the treatment of cancers, preferably HER2-positive gastric adenocarcinoma or a gastroesophageal junction adenocarcinoma in an individual.

### CROSS-REFERENCE TO RELATED APPLICATIONS

This application is the U.S. national stage application of International Patent Application No. PCT/EP2021/072020, filed Aug. 6, 2021, which claims the benefit of U.S. Provisional Application No. US 63/065,085 filed 13 Aug. 2020, which is incorporated herein by reference in its entirety, including any drawings.

### REFERENCE TO SEQUENCE LISTING

The present application is being filed along with a Sequence Listing in electronic format. The Sequence Listing is provided as a file entitled “Seq-List-replace.txt”, created Aug. 2, 2023, which is 81,940 bytes in size. The information in the electronic format of the Sequence Listing is incorporated herein by reference in its entirety.

### FIELD OF THE DISCLOSURE

The disclosure relates to methods and compositions for the treatment of cancer. Specifically, the disclosure relates to methods for administering anti-CD73 antibodies, and to methods for using anti-CD73 antibodies for the treatment of cancers.

### BACKGROUND

CD73 (ecto-5′-nucleotidase) is a 70-kDa glycosylphosphatidylinositol (GPI)-anchored protein normally expressed on endothelial cells and subsets of hematopoietic cells. CD73, together with CD39, regulates adenosine triphosphate (ATP) metabolism. CD39 (NTPDase-1) converts ATP into AMP, with only trace amounts of ADP being released, while CD73 catalyzes the conversion of AMP to adenosine.

Adenosine triphosphate (ATP) and its metabolites AMP and adenosine, have important roles in cellular metabolism, signaling and immune homeostasis. The release of extracellular adenosine triphosphates (ATP) in response to cell death or cellular stress acts to activate immune responses. However, its metabolite adenosine has immunosuppressive activity. Extracellular adenosine accumulates in cancerous tissues and constitutes an important mechanism of tumor immune escape. Among other effects, tumor-derived adenosine profoundly inhibits infiltrating effector T cells through adenylyl cyclase-activating A2A receptors.

CD73 expression has been reported in a range of tumor cells, including leukemia, bladder cancer, glioma, glioblastoma, ovarian cancer, melanoma, prostate cancer, thyroid cancer, esophageal cancer and breast cancer. CD73 expression has also been associated with a prometastatic phenotype in melanoma and breast cancer. It has been reported that therapy with an antibody that binds murine CD73 can inhibit breast tumor growth and metastasis in mice (Stagg, et al. (2010) Proc. Natl. Acad. Sci. USA 104:1547-1552). It has been shown that genetic deletion of A2A receptors can induce T cell-dependent tumor rejection (Ohta, et al., (2006) Proc Natl Acad Sci USA 103:13132-13137). Knock-down using siRNA or overexpression of CD73 on tumor cells can modulate tumor growth and metastasis (Beavis et al. (2013 Proc. Natl. Acad. Sci. USA 110:14711-716; Stagg et al. (2010), supra; Jin et al. (2010) Cancer Res. 70: 2245-55). CD73-/- mice are protected from transplanted and spontaneous tumors (Stagg et al. (2010) Cancer Res. 71: 2892-2900). In humans, high CD73 expression had been shown to be a negative prognostic for triple negative breast cancer (Loi et al. (2013) Proc. Natl. Acad. Sci. USA 110: 11091-11096).

Development of anti-CD73 antibodies is complex. While CD73 is expressed on tumor cells, it is also expressed on different cells of the immune system, notably CD4 and CD8 T cells, as well as B cells. One further complicating factor is that many of the antibodies described in the literature vary greatly in their activities and modes of action. Many of the initial reports relating to CD73 made use of small molecule inhibitors which may lack specificity and/or may be too toxic for in vivo studies. Many reports with anti-CD73 antibodies have generally been of murine isotypes that are capable of being bound by Fcγ receptors, making it difficult to separate any potential blocking effect from Fc-mediated effects. More recently, various therapeutic anti-CD73 antibodies have been reported. Some of these function by causing intracellular internalization of CD73, others inhibit membrane bound CD73 with differing degrees of efficacy and moreover with or without the ability to inhibit soluble CD73 protein.

Around 12-20% of patients with gastric cancer in the Western world overexpress the HER2 oncogene (Jan B et al.; J Clin Pathol 2010 & Tanner M et al.; Ann Oncol 2005). While trastuzumab in combination with chemotherapy is considered an option in patients with advanced metastatic HER2-positive disease. However, unlike in breast cancer, in gastric cancer the introduction of trastuzumab did not result in changing the natural history of the disease with median survival hardly exceeding one year. There is therefore for improved treatment regimens that provide maximal efficacy for the treatment of gastric cancers.

### SUMMARY OF THE INVENTION

In one embodiment, provided herein is use of an antibody that neutralizes the 5′-ectonucleotidase activity of human CD73 protein for the treatment of a HER2-positive cancer, optionally a gastric adenocarcinoma or a gastroesophageal junction adenocarcinoma, characterized by tumor cells that express HER2 at their surface.

In one embodiment, provided herein is a method of reducing or inhibiting the growth of a gastric cancer (e.g., a gastric adenocarcinoma or a gastroesophageal junction adenocarcinoma) in an individual in need thereof, comprising administering to the individual a therapeutically effective amount of each of an antibody that neutralizes the 5′-ectonucleotidase activity of human CD73 protein and an antibody that binds a human HER2 protein. Optionally, the method further comprises administering to the individual a therapeutically effective amount of a chemotherapy agent. In one embodiment, the cancer is a HER2-positive cancer. In one embodiment, the tumor or cancer is characterized by tumor cells that express at their surface HER2 protein. In one embodiment, the tumor or cancer is characterized by a tumor stroma that comprises cells that express CD73 protein at their surface.

Further provided herein is a method of treating cancer, in particular gastric cancer, in an individual in need thereof, comprising administering to the individual a therapeutically effective amount of each of an antibody that neutralizes the 5′-ectonucleotidase activity of human CD73 protein and an antibody that binds a human HER2 protein. Optionally, the method further comprises administering to the individual a therapeutically effective amount of a chemotherapy agent. In one embodiment, the tumor or cancer is characterized by tumor cells that express at their surface HER2 protein. In one embodiment, the tumor or cancer is characterized by a tumor stroma that comprises cells that express CD73 protein at their surface.

Further provided herein is a method of sensitizing an individual having a cancer, in particular gastric cancer, to treatment with an antibody that binds a human HER2 protein, the method comprising administering to the individual a therapeutically effective amount of an antibody that neutralizes the 5′-ectonucleotidase activity of human CD73 protein. Optionally, the method further comprises administering to the individual a therapeutically effective amount of the antibody that binds a human HER2 protein. In one embodiment, the antibody that neutralizes the 5′-ectonucleotidase activity of human CD73 protein is administered on the same day and/or before the antibody that binds a human HER2 protein.

Further provided herein is a method of sensitizing an individual having a cancer, particularly a gastric cancer, to treatment with an antibody that binds a human HER2 protein and/or a chemotherapy agent, wherein the individual is being treated with (or who is to be treated with) a chemotherapy agent and an antibody that binds a human HER2 protein, the method comprising administering to the individual an antibody that neutralizes the 5′-ectonucleotidase activity of human CD73 protein. In one embodiment, the method further comprises administering to the individual an antibody that binds a human HER2 protein and a chemotherapy agent. In one embodiment, the antibody that neutralizes the 5′-ectonucleotidase activity of human CD73 protein is administered on the same day and/or before the administration of the antibody that binds a human HER2 protein, optionally further on the same day and/or before the administration of the chemotherapy agent.

In any embodiment, the first administration of anti-CD73 antibody occurs before the first administration of the anti-HER2 antibody.

In one embodiment, a treatment comprises a plurality of administrations of anti-HER2 antibody and a plurality of administrations of anti-CD73 antibody, wherein each administration of anti-CD73 antibody occurs between 1 hour and 7 days before an administration of anti-HER2 antibody. In one embodiment, a treatment comprises a plurality of administrations of anti-HER2 antibody and a plurality of administrations of anti-CD73 antibody, wherein each administration of anti-HER2 antibody is preceded by 1 hour to 7 days by an administration of anti-CD73 antibody. For example the anti-CD73 antibody is administered either on the same day (e.g. 1-12 hours before the anti-HER2 antibody) or within one week preceding the administration of the anti-HER2 antibody. In one embodiment, each of the plurality of administrations of anti-CD73 antibody occur either on the same day (e.g. 1-12 hours before the anti-HER2 antibody) or about one week (e.g., 7 days) before an administration of the anti-HER2 antibody. In one embodiment, each of the plurality of administrations of anti-HER2 antibody occur either on the same day (e.g. 1-12 hours after administration of an anti-CD73 antibody) or about one week (e.g., 7 days) after an administration of an anti-CD73 antibody.

In any embodiment of a combination treatment comprising a chemotherapy agent, the first administration of anti-CD73 antibody can be specified to occur before the first administration of chemotherapy agent (e.g., optionally further before the first administration of the anti-HER2 antibody).

In one embodiment, a treatment comprises a plurality of administrations of anti-HER2 antibody, a plurality of administrations of chemotherapy agent and a plurality of administrations of anti-CD73 antibody, wherein each administration of anti-CD73 antibody occurs between 1 hour and 7 days before an administration of anti-HER2 antibody, and further wherein each administration of anti-CD73 antibody occurs between 1 hour and 7 days before an administration of chemotherapy agent. In one embodiment, a treatment comprises a plurality of administrations of anti-HER2 antibody, a plurality of administrations of chemotherapy agent and a plurality of administrations of anti-CD73 antibody, wherein each administration of anti-HER2 antibody is preceded by 1 hour to 7 days by an administration of anti-CD73 antibody and wherein each administration of chemotherapy agent is preceded by 1 hour to 7 days by an administration of anti-CD73 antibody. For example the anti-CD73 antibody is administered either on the same day (e.g. 1-12 hours before the chemotherapy agent) or within one week preceding the administration of the chemotherapy agent. In one embodiment, each of the plurality of administrations of anti-CD73 antibody occur either on the same day (e.g. 1-12 hours before the chemotherapy agent) or about one week (e.g., 7 days) before an administration of the chemotherapy agent. In one embodiment, each of the plurality of administrations of chemotherapy agent and each of the administrations of ant-HER2 antibody occur either on the same day as administration of an anti-CD73 antibody (e.g. 1-12 hours after administration of an anti-CD73 antibody) or about one week (e.g., 7 days) after an administration of an anti-CD73 antibody.

Optionally, the plurality of administrations of anti-HER2 antibody and the plurality of administrations of anti-CD73 antibody occur on the same day. In one embodiment, the anti-HER2 antibody is administered subcutaneously every three weeks and the anti-CD73 antibody is administered every three weeks, wherein the anti-HER2 antibody and anti-CD73 antibody are administered on the same day. In one embodiment, the anti-HER2 antibody is administered intravenously every three weeks and the anti-CD73 antibody is administered every three weeks, wherein the anti-HER2 antibody and anti-CD73 antibody are administered on the same day.

In one embodiment, the plurality of administrations of chemotherapy agent and the plurality of administrations of anti-HER2 antibody are each administered every 3 weeks in a staggered schedule such that the anti-HER2 antibody and of chemotherapy agent are not dosed on the same day (e.g. administrations of anti-HER2 antibody and chemotherapy are separated by at least one week), and such that each administration of anti-HER2 antibody and each administration of chemotherapy agent is preceded by 1 hour to 7 days by an administration of anti-CD73 antibody.

In any embodiment, the tumor or cancer is characterized by tumor cells that express (e.g., overexpress) at their surface HER2 protein. In one embodiment, the tumor or cancer is characterized by a tumor stroma that comprises cells that express CD73 protein at their surface.

In any embodiment, the antibody that neutralizes the 5′-ectonucleotidase activity of human CD73 protein is capable of specifically binding to a human CD73 polypeptide. In one embodiment, the antibody is an antibody fragment. In one embodiment, the antibody is a full-length antibody. In one embodiment, the antibody is capable of neutralizing the 5′-ectonucleotidase activity of a soluble human CD73 polypeptide. In one embodiment, the antibody binds a CD73 polypeptide dimer in bivalent manner; for example the antibody is capable of neutralizing the 5′-ectonucleotidase activity of a soluble human dimeric CD73 polypeptide when the antibody is provided at a 10-fold of greater molar excess to CD73 polypeptide dimer.

In one embodiment, the antibody that binds a human HER2 protein comprises the heavy and light chain CDR1, 2 and 3 regions of trastuzumab. In one embodiment, CDR are determined according to Kabat numbering. In one embodiment, the antibody that binds a human HER2 protein comprises the heavy and light chain variable regions of trastuzumab.

In one embodiment, the chemotherapy agent is an agent that induces the extracellular release of ATP from tumor cells, for example the chemotherapy agent is capable of inducing immunogenic cancer cell death. In one embodiment, the chemotherapy agent is a taxane, for example paclitaxel or an analogue thereof. In one embodiment, the chemotherapy agent is a camptothecin analogue or derivative thereof, for example SN-38, exatecan or exatecan derivative (e.g., DX-8951 derivative, DXd; see Ogitano et al. (2016) Cancer Sci 107 (2016) 1039-1046). In one embodiment, the chemotherapy agent (e.g. paclitaxel) is administered as free chemotherapy agent (not conjugated or covalently bound to another agent (e.g. antibody or active agent). In another embodiment, the chemotherapy agent (e.g., a camptothecin analogue or derivative) is administered as an antibody drug conjugate (ADC) in which the chemotherapy agent is conjugated (e.g. covalently bound) to an antibody that binds to a HER2 protein.

In one aspect, provided is a treatment comprising administering to an individual having a HER2-positive cancer (e.g., a gastric cancer) an antibody that neutralizes the 5′-ectonucleotidase activity of human CD73 protein and an antibody that binds a human HER2 protein, optionally further in combination with a chemotherapy agent, e.g. a taxane. In one embodiment, the HER2-positive cancer is a cancer known to be generally characterized by HER2 expression at the surface of tumor cells. In one embodiment, the HER2-positive cancer is a cancer known to be generally characterized by presence of soluble CD73 protein and/or CD73-expressing cells in the tumor or tumor environment (e.g. in tumor cell or in cells in the tumor stromal tissue). In one embodiment, the HER2-positive cancer is characterized by the presence of HER2-expressing tumor cells, for example as assessed by immunohistochemistry (IHC) using an anti-HER2 antibody, optionally wherein the tumor comprises increased numbers compared to a reference (e.g. healthy tissue) or frequencies of HER2-expressing cells and/or stronger HER2 staining intensity compared to a reference (e.g. healthy tissue). In one embodiment, a cancer is characterized by CD73-expressing tumor cells and/or CD73-expressing cells in the tumor stromal tissue, for example as assessed by immunohistochemistry using an anti-CD73 antibody, optionally wherein the tumor comprises increased numbers compared to a reference (e.g. healthy tissue) or frequencies of CD73-expressing cells and/or stronger CD73 staining intensity compared to a reference (e.g. healthy tissue).

In one embodiment, the individual having a HER2-positive cancer is treated with an antibody that neutralizes the 5′-ectonucleotidase activity of human CD73 protein, an antibody that binds a human HER2 protein, and a chemotherapeutic agent that induces the extracellular release of ATP from tumor cells, notably agents or treatments that induce immunogenic cancer cell death (e.g., a taxane agent, paclitaxel).

In another aspect, provided herein is a method of treating an individual having a HER2-positive gastric cancer wherein the HER2-positive gastric cancer is characterized by a tumor determined to comprise HER2-expressing cells, the method comprising administering to the individual a means for binding and inhibiting the 5′-ectonucleotidase activity of human CD73 protein (or a pharmaceutical comprising such means). Optionally, the antibody that is capable of binding and inhibiting the 5′-ectonucleotidase activity of human CD73 protein is administered in combination with an antibody that binds a human HER2 protein, and optionally further a chemotherapeutic agent that induces the extracellular release of ATP from tumor cells, notably agents or treatments that induce immunogenic cancer cell death (e.g., a taxane agent, paclitaxel).

In another aspect, provided herein is, in a method of treating an individual having cancer with an antibody that binds CD73, the improvement comprising identifying an individual having a HER2-positive gastric cancer characterized by a gastric tumor determined to comprise HER2-expressing cells and administering to the individual an antibody that is capable of binding and inhibiting the 5′-ectonucleotidase activity of human CD73 protein. Optionally, the antibody that is capable of binding and inhibiting the 5′-ectonucleotidase activity of human CD73 protein is administered in combination with an antibody that binds a human HER2 protein, and optionally further a chemotherapeutic agent that induces the extracellular release of ATP from tumor cells, notably agents or treatments that induce immunogenic cancer cell death (e.g., a taxane agent, paclitaxel).

In another aspect, provided herein is, in a method of treating an individual having cancer with an antibody that binds CD73, the improvement comprising identifying an individual having a HER2-positive gastric cancer characterized by a gastric tumor determined to comprise HER2-expressing cells and administering to the individual, in addition to the antibody that is capable of binding, and inhibiting the 5′-ectonucleotidase activity of, human CD73 protein, an antibody that binds a human HER2 protein, and optionally further a chemotherapeutic agent that induces the extracellular release of ATP from tumor cells, notably agents or treatments that induce immunogenic cancer cell death (e.g., a taxane agent, paclitaxel).

In any embodiment herein, identifying an individual having a HER2-positive gastric cancer can comprise assessing whether cancer cells in a biological sample express HER2, as determined by IHC and/or FISH.

In any embodiment herein, the individual having a HER2-positive cancer has received previous treatment (e.g. at least one previous course or cycle of treatment) with a chemotherapeutic agent that induces the extracellular release of ATP from tumor cells, notably agents or treatments that induce immunogenic cancer cell death (e.g., a taxane agent, paclitaxel). Optionally, the individual has a tumor or cancer that has relapsed following such previous treatment.

In any embodiment herein, the individual having a HER2-positive cancer has received previous treatment (e.g. at least one previous course or cycle of treatment) with an antibody that binds a human HER2 protein. Optionally, the individual has a tumor or cancer that has relapsed following such previous treatment. In further aspects, the disclosure provides advantageous 2-weekly or 3-weekly treatment regimens for administering anti-CD73 antibodies; such regimens can be particularly useful for treatment with an antibody having the light chain variable region having the amino acid sequence of SEQ ID NO: 43 and a heavy chain variable region having the amino acid sequence of SEQ ID NO: 42, or a function-conservative variant thereof. The anti-CD73 antibodies administered in such a treatment regimen can be administered in combined treatments with other therapeutic agents (e.g. a chemotherapeutic agent and/or an anti-HER2 antibody) or without combined treatment with other therapeutic agents (as monotherapy). In one aspect, provided is a method of enhancing, potentiating or inducing an anti-tumor immune response in an individual, a method of relieving immunosuppression (e.g. tumor-mediated immunosuppression) in an individual, a method of neutralizing the activity of CD73 in the tumor microenvironment, a method of decreasing the generation and/or concentration of adenosine in the tumor microenvironment, a method of neutralizing the activity of CD73 expressed by tumor cells, CD4 T cells, CD8 T cells and/or B cells, a method of potentiating the activity of lymphocytes (e.g., T cells) in an individual, or for restoring the activity of lymphocytes (e.g., T cells), and/or a method of relieving the adenosine-mediated inhibition of lymphocyte activity (e.g., T cells) in an individual, the method comprising administering to the individual an antibody disclosed herein that binds a human CD73 protein and neutralizes the 5′-ectonucleotidase activity thereof (e.g. an antibody or antibody fragment comprising a light chain variable region having the amino acid sequence of SEQ ID NO: 43 and a heavy chain variable region having the amino acid sequence of SEQ ID NO: 42, or a function-conservative variant thereof), wherein the antibody is administered to the individual (a) once every two weeks at a fixed dose (irrespective of body weight or surface area) of 1500-3600 mg, optionally at a dose of 1500 mg, optionally at a dose of 2400 mg, or (b) once every three weeks at a fixed dose of 2000-3000 mg, optionally at a dose of 2100 mg, 2200 mg, 2300 mg, 2400 mg, 2500 mg, 2600 mg, 2700 mg, 2800 mg, 2900 mg or 3000 mg.

In one aspect, provided is a method of treating a cancer in an individual, comprising administering to the individual an antibody that binds a human CD73 protein and neutralizes the 5′-ectonucleotidase activity thereof, wherein the antibody is an antibody or antibody fragment comprising a light chain variable region having the amino acid sequence of SEQ ID NO: 43 and a heavy chain variable region having the amino acid sequence of SEQ ID NO: 42, or a function-conservative variant thereof, wherein the antibody is administered to the individual once every two weeks at a fixed dose (irrespective of body weight or surface area) of 1500-3600 mg, optionally at a dose of 1500 mg, optionally at a dose of 2400 mg, or once every three weeks at a fixed dose of 2000-3000 mg, optionally at a dose of 2100 mg, 2200 mg, 2300 mg, 2400 mg, 2500 mg, 2600 mg, 2700 mg, 2800 mg, 2900 mg or 3000 mg.

These aspects are more fully described in, and additional aspects, features, and advantages will be apparent from, the description provided herein.

### BRIEF DESCRIPTION OF THE DRAWINGS

FIG. 1 left panel: CD73 staining intensity in gastric primary tumors at diagnosis (before any treatment). Staked bars showing the distribution of CD73 intensity scores on tumor cells (TC) and stromal cells (SC) expressing CD73. Intensity score from 1 to 3. FIG. 1 right panel: CD73 expression in gastric tumor cells at diagnosis according to Her2 status. Staked bars showing the distribution of CD73 proportion scores on tumors cells. Statistical analysis: nonparametric Mann-Whitney test (*p=0.0343). Score 0=no positive cells; Score 1=1-9% of positive cells; Score 2=10-50% of positive cells; Score 3=51-80% of positive cells, and Score 4=>80% positive cells.

FIG. 2: Comparison of CD73-expressing gastric tumor cells pre- and post-neoadjuvant chemotherapy. Staked bars (left panel) showing the distribution of CD73 proportion scores on tumor cells and violin plots (right panel) showing the percentages of CD73+ tumor cells. Score 0=no positive cells; Score 1=1-9% of positive cells; Score 2=10-50% of positive cells; Score 3=51-80% of positive cells, and Score 4=>80% positive cells.

FIG. 3: Comparison of CD73-expressing gastric stromal cells pre- and post-neoadjuvant chemotherapy. Staked bars (left panel) showing the distribution of CD73 proportion scores on stromal cells and violin plots (right panel) showing the percentages of CD73+ stromal cells. Score 0=no positive cells; Score 1=1-9% of positive cells; Score 2=10-50% of positive cells; Score 3=51-80% of positive cells, and Score 4=>80% positive cells.

FIG. 4: Comparison of CD73-expressing gastric stromal immune cells pre- and post-neoadjuvant chemotherapy. Staked bars (left panel) showing the distribution of CD73 proportion scores on stromal immune cells and violin plots (right panel) showing the percentages of CD73+ stromal immune cells. Score 0=no positive cells; Score 1=1-9% of positive cells; Score 2=10-50% of positive cells; Score 3=51-80% of positive cells, and Score 4=>80% positive cells.

FIGS. 5A-5C show T cell proliferation is restored by all anti-CD73 Abs. FIG. 5A shows control of T cell sub-population proliferation and its inhibition by AMP. FIG. 5B shows efficacy of H4+Lx antibodies and parental antibody HPLP to restore CD4+ and CD8+ T cell proliferation. FIG. 5C shows efficacy of 2H4+2Lx antibodies (the 2H4+ chain combined with 2L1, 2L2, 2L3 or 2L4 chains) and parental antibody 2HP2LP to restore CD4+ and CD8+ T cell proliferation. Data are expressed as mean of duplicates +/− standard deviation.

FIGS. 6A and 6B show T cell proliferation restored by humanized variants is reproducible, respectively in two representative human donors. Shown is efficacy of 2H4+2Lx anti-CD73 antibody variants to restore CD4 and CD8 T cell proliferation. Data are expressed as mean of duplicates +/− standard deviation.

FIG. 7 shows CD4+ T cell proliferation is inhibited by ATP and restored with 2H4+2Lx antibody variants. Control of CD4+ T cell proliferation and inhibition by 100 μM of ATP, shown as tested for two representative donors, D795 (FIG. 7, top panel) and D664 (FIG. 7, bottom panel). Data are expressed as mean of duplicates +/− standard deviation.

FIG. 8 shows a pharmacokinetics/pharmacodynamics model for anti-CD73 antibody.

The model includes: two-compartment distribution (from blood to periphery), characterized by an inter-compartmental clearance (Q) and distribution volumes for the central and peripheral compartments (respectively Vc and Vp); First order elimination from the central compartment, characterized by a single clearance parameter CL; Michaelis Menten saturable elimination from the central compartment, characterized by Vmax and Km. CD73 saturation levels for the PD model are projected from the Km, representing EC50, the anti-CD73 antibody serum concentration leading to 50% receptor occupancy.

FIG. 9 shows the overlay of predicted and observed anti-CD73 antibody serum concentrations in non-GLP and GLP Toxicology studies in cynomolgus monkeys. The left-hand Panel represents a model fitting to observed PK data from non-GLP toxicology study; the right hand Panel shows the model fitting to observed PK data from GLP toxicology study; Symbols represent the observed data; solid lines represent the model prediction.

FIG. 10A shows the predicted anti-CD73 antibody serum concentrations for administration every two weeks—time and CD73 occupancy—time profiles. FIG. 10B shows the predicted anti-CD73 antibody serum concentrations for administration every three weeks. Left hand panels shows predicted anti-CD73 antibody PK in humans. Right hand panels shows predicted CD73 receptor occupancy in humans. EC50 was the Michaelis-Menten constant Km estimated by PK modeling. EC90 was calculated from EC50. Lines from bottom to top correspond to the increasing doses of anti-CD73 antibody.

### DETAILED DESCRIPTION OF THE INVENTION

### Definitions

As used in the specification, “a” or “an” may mean one or more. As used in the claim(s), when used in conjunction with the word “comprising”, the words “a” or “an” may mean one or more than one. As used herein “another” may mean at least a second or more.

Where “comprising” is used, this can optionally be replaced by “consisting essentially of” or by “consisting of”.

Human CD73, also known as ecto-5′-nucleotidase and as 5-prime-ribonucleotide phosphohydrolase, EC 3.1.3.5, encoded by the NT5E gene, exhibits 5′-nucleotidase, notably AMP-, NAD-, and NMN-nucleosidase, activities. CD73 catalyzes the conversion at neutral pH of purine 5-prime mononucleotides to nucleosides, the preferred substrate being AMP. The enzyme consists of a dimer of 2 identical 70-kD subunits bound by a glycosyl phosphatidyl inositol linkage to the external face of the plasma membrane The amino acid sequence of Human CD73 preprotein (monomer), including a signal sequence at amino acids 1-26, is shown in Genbank under accession number NP_002517, the entire disclosure of which is incorporated herein by reference, and as follows:

(SEQ ID NO: 1)
MCPRAARAPA TLLLALGAVL WPAAGAWELT ILHTNDVHSR
LEQTSEDSSK CVNASRCMGG VARLFTKVQQ IRRAEPNVLL
LDAGDQYQGT IWFTVYKGAE VAHFMNALRY DAMALGNHEF
DNGVEGLIEP LLKEAKFPIL SANIKAKGPL ASQISGLYLP
YKVLPVGDEV VGIVGYTSKE TPFLSNPGTN LVFEDEITAL
QPEVDKLKTL NVNKIIALGH SGFEMDKLIA QKVRGVDVVV
GGHSNTFLYT GNPPSKEVPA GKYPFIVTSD DGRKVPVVQA
YAFGKYLGYL KIEFDERGNV ISSHGNPILL NSSIPEDPSI
KADINKWRIK LDNYSTQELG KTIVYLDGSS QSCRFRECNM
GNLICDAMIN NNLRHTDEMF WNHVSMCILN GGGIRSPIDE
RNNGTITWEN LAAVLPFGGT FDLVQLKGST LKKAFEHSVH
RYGQSTGEFL QVGGIHVVYD LSRKPGDRVV KLDVLCTKCR
VPSYDPLKMD EVYKVILPNF LANGGDGFQM IKDELLRHDS
GDQDINVVST YISKMKVIYP AVEGRIKFST GSHCHGSFSL
IFLSLWAVIF VLYQ.

In the context herein, “neutralize the enzymatic activity of CD73”, refers to a process in which the 5′-nucleotidase (5′-ectonucleotidase) activity of CD73 is inhibited. This comprises, notably the inhibition of CD73-mediated generation of adenosine, i.e. the inhibition of CD73-mediated catabolismof AMP to adenosine. This can be measured for example in a cell-free assay that measures the capacity of a test compound to inhibit the conversion of AMP to adenosine, either directly or indirectly. In one embodiment, an antibody preparation causes at least a 50% decrease in the conversion of AMP to adenosine, at least a 70% decrease in the conversion of AMP to adenosine, or at least an 80% decrease in the conversion of AMP to adenosine, referring, for example, to the assays described herein.

Whenever within this whole specification “treatment of cancer” or the like is mentioned with reference to anti-CD73 antibody, there is meant: (a) method of treatment of cancer, said method comprising the step of administering (for at least one treatment) an anti-CD73 antibody, (preferably in a pharmaceutically acceptable carrier material) to an individual, a mammal, especially a human, in need of such treatment, in a dose that allows for the treatment of cancer, (a therapeutically effective amount), preferably in a dose (amount) as specified herein; (b) the use of an anti-CD73 antibody for the treatment of cancer, or an anti-CD73 antibody, for use in said treatment (especially in a human); (c) the use of an anti-CD73 antibody for the manufacture of a pharmaceutical preparation for the treatment of cancer, a method of using an anti-CD73 antibody for the manufacture of a pharmaceutical preparation for the treatment of cancer, comprising admixing an anti-CD73 antibody with a pharmaceutically acceptable carrier, or a pharmaceutical preparation comprising an effective dose of an anti-CD73 antibody that is appropriate for the treatment of cancer; or (d) any combination of a), b), and c), in accordance with the subject matter allowable for patenting in a country where this application is filed.

The term “antibody,” as used herein, refers to polyclonal and monoclonal antibodies. Depending on the type of constant domain in the heavy chains, antibodies are assigned to one of five major classes: IgA, IgD, IgE, IgG, and IgM. Several of these are further divided into subclasses or isotypes, such as IgG1, IgG2, IgG3, IgG4, and the like. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids that is primarily responsible for antigen recognition. The terms variable light chain (VL) and variable heavy chain (VH) refer to these light and heavy chains respectively. The heavy-chain constant domains that correspond to the different classes of immunoglobulins are termed “alpha,” “delta,” “epsilon,” “gamma” and “mu,” respectively. The subunit structures and three-dimensional configurations of different classes of immunoglobulins are well known. IgG are the exemplary classes of antibodies employed herein because they are the most common antibodies in the physiological situation and because they are most easily made in a laboratory setting. Optionally the antibody is a monoclonal antibody. Particular examples of antibodies are humanized, chimeric, human, or otherwise-human-suitable antibodies. “Antibodies” also includes any fragment or derivative of any of the herein described antibodies.

The term “specifically binds to” means that an antibody can bind preferably in a competitive binding assay to the binding partner, e.g., CD73, as assessed using either recombinant forms of the proteins, epitopes therein, or native proteins present on the surface of isolated target cells. Competitive binding assays and other methods for determining specific binding are further described below and are well known in the art.

When an antibody is said to “compete with” a particular monoclonal antibody, it means that the antibody competes with the monoclonal antibody in a binding assay using either recombinant CD73 molecules or surface expressed CD73 molecules. For example, if a test antibody reduces the binding of a reference antibody to a CD73 polypeptide or CD73-expressing cell in a binding assay, the antibody is said to “compete” respectively with the reference antibody.

The term “affinity”, as used herein, means the strength of the binding of an antibody to an epitope. The affinity of an antibody is given by the dissociation constant Kd, defined as [Ab]×[Ag]/[Ab-Ag], where [Ab-Ag] is the molar concentration of the antibody-antigen complex, [Ab] is the molar concentration of the unbound antibody and [Ag] is the molar concentration of the unbound antigen. The affinity constant Ka is defined by 1/Kd. Methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y. (1992, 1993), and Muller, Meth. Enzymol. 92:589-601 (1983), which references are entirely incorporated herein by reference. One standard method well known in the art for determining the affinity of mAbs is the use of surface plasmon resonance (SPR) screening (such as by analysis with a BIAcore™ SPR analytical device).

Within the context herein a “determinant” designates a site of interaction or binding on a polypeptide.

The term “epitope” refers to an antigenic determinant, and is the area or region on an antigen to which an antibody binds. A protein epitope may comprise amino acid residues directly involved in the binding as well as amino acid residues which are effectively blocked by the specific antigen binding antibody or peptide, i.e., amino acid residues within the “footprint” of the antibody. It is the simplest form or smallest structural area on a complex antigen molecule that can combine with e.g., an antibody or a receptor. Epitopes can be linear or conformational/structural. The term “linear epitope” is defined as an epitope composed of amino acid residues that are contiguous on the linear sequence of amino acids (primary structure). The term “conformational or structural epitope” is defined as an epitope composed of amino acid residues that are not all contiguous and thus represent separated parts of the linear sequence of amino acids that are brought into proximity to one another by folding of the molecule (secondary, tertiary and/or quaternary structures). A conformational epitope is dependent on the 3-dimensional structure. The term ‘conformational’ is therefore often used interchangeably with ‘structural’.

The term “deplete” or “depleting”, with respect to CD73-expressing cells, means a process, method, or compound that results in killing, elimination, lysis or induction of such killing, elimination or lysis, so as to negatively affect the number of such CD73-expressing cells present in a sample or in a subject.

The term “internalization”, used interchangeably with “intracellular internalization”, refers to the molecular, biochemical and cellular events associated with the process of translocating a molecule from the extracellular surface of a cell to the intracellular surface of a cell. The processes responsible for intracellular internalization of molecules are well-known and can involve, inter alia, the internalization of extracellular molecules (such as hormones, antibodies, and small organic molecules); membrane-associated molecules (such as cell-surface receptors); and complexes of membrane-associated molecules bound to extracellular molecules (for example, a ligand bound to a transmembrane receptor or an antibody bound to a membrane-associated molecule). Thus, “inducing and/or increasing internalization” comprises events wherein intracellular internalization is initiated and/or the rate and/or extent of intracellular internalization is increased.

The term “agent” is used herein to denote a chemical compound, a mixture of chemical compounds, a biological macromolecule, or an extract made from biological materials. The term “therapeutic agent” refers to an agent that has biological activity.

For the purposes herein, a “humanized” or “human” antibody refers to an antibody in which the constant and variable framework region of one or more human immunoglobulins is fused with the binding region, e.g., the CDR, of an animal immunoglobulin. Such antibodies are designed to maintain the binding specificity of the non-human antibody from which the binding regions are derived, but to avoid an immune reaction against the non-human antibody. Such antibodies can be obtained from transgenic mice or other animals that have been “engineered” to produce specific human antibodies in response to antigenic challenge (see, e.g., Green et al. (1994) Nature Genet 7:13; Lonberg et al. (1994) Nature 368:856; Taylor et al. (1994) Int Immun 6:579, the entire teachings of which are herein incorporated by reference). A fully human antibody also can be constructed by genetic or chromosomal transfection methods, as well as phage display technology, all of which are known in the art (see, e.g., McCafferty et al. (1990) Nature 348:552-553). Human antibodies may also be generated by in vitro activated B cells (see, e.g., U.S. Pat. Nos. 5,567,610 and 5,229,275, which are incorporated in their entirety by reference).

The term “hypervariable region” when used herein refers to the amino acid residues of an antibody that are responsible for antigen binding. The hypervariable region generally comprises amino acid residues from a “complementarity-determining region” or “CDR” (e.g., residues 24-34 (L1), 50-56 (L2) and 89-97 (L3) in the light-chain variable domain and 31-35 (H1), 50-65 (H2) and 95-102 (H3) in the heavy-chain variable domain; Kabat et al. 1991) and/or those residues from a “hypervariable loop” (e.g., residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987; 196:901-917), or a similar system for determining essential amino acids responsible for antigen binding. Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

By “framework” or “FR” residues as used herein is meant the region of an antibody variable domain exclusive of those regions defined as CDRs. Each antibody variable domain framework can be further subdivided into the contiguous regions separated by the CDRs (FR1, FR2, FR3 and FR4).

The terms “Fc domain,” “Fc portion,” and “Fc region” refer to a C-terminal fragment of an antibody heavy chain, e.g., from about amino acid (aa) 230 to about aa 450 of human γ (gamma) heavy chain or its counterpart sequence in other types of antibody heavy chains (e.g., α, δ, ϵ and μ for human antibodies), or a naturally occurring allotype thereof. Unless otherwise specified, the commonly accepted Kabat amino acid numbering for immunoglobulins is used throughout this disclosure (see Kabat et al. (1991) Sequences of Protein of Immunological Interest, 5th ed., United States Public Health Service, National Institute of Health, Bethesda, MD).

The terms “isolated”, “purified” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under expressed or not expressed at all.

Within the context herein, the term antibody that “binds” a polypeptide or epitope designates an antibody that binds said determinant with specificity and/or affinity.

The term “identity” or “identical”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Methods for determining identity are designed to give the largest match between the sequences tested. Methods of determining identity are described in publicly available computer programs. Computer program methods for determining identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

### Treatment Methods

Described are methods useful in the diagnosis, prognosis, monitoring and treatment of cancer. In one embodiment, the cancer is characterized by tumor cells that express HER2 at their surface, wherein the treatment comprises administering to an individual an anti-CD73 antibody as described herein. In one embodiment, an individual treated according to the disclosure has a gastric cancer. In one embodiment, an individual treated according to the disclosure has a breast cancer. In any embodiment herein, a gastric cancer can be characterized as being a gastric adenocarcinoma or a gastroesophageal or gastroesophageal junction adenocarcinoma. In any embodiment, the cancer can be characterized as testing (or having tested) positive for HER2 protein, optionally wherein the cancer expresses excess HER2 protein (HER2 over-expression), optionally wherein the cancer expresses low levels of HER2 protein (lower than excess HER2 expression or HER2 overexpression).

As shown herein, patient stratification according to HER2 status in gastric cancer showed that all HER2+ gastric cancer patients had CD73-expressing tumors, and that Her2+ patients had a higher percentage of CD73+ tumor cells than Her2-patients. All patients had CD73+ stromal cells irrespective of Her2 status. Yet further, as shown herein, neoadjuvant chemotherapy (CT) did not significantly modify CD73-expressing tumor cells either in the percentage of patients expressing CD73 or in the percentage of CD73+ tumor cells. However, neoadjuvant CT increased the percentage of patients with CD73+ stromal cells from 60% pre-CT (n=12/20) to 95% post-CT (n=19/20). The percentage of CD73-expressing stromal cells was not significantly different pre- and post-CT (FIG. 15, right panel). CT increased the percentage of patients with CD73+ stromal immune cells from 30% (n=6/20) to 70% (n=14/20). The anti-CD73 antibody can therefore be used advantageously in individuals who have previously received treatment with a chemotherapy agent. Yet further, the lack of effect of chemotherapy on the percentage of CD73-expressing cells in CD73-positive samples may provide that a treatment regimen and dosage of anti-CD73 antibody (e.g. a dose designed to saturate CD73 receptors) will be effective including when administered in combination with treatment with a chemotherapy agent.

The term “HER2” (also known as HER2/neu, C-erbB-2 and ErbB-2) stands for “Human Epidermal growth factor Receptor 2”. HER2, with its variants and isoforms, is a proto-oncogene located on chromosome 17q21 that encodes a transmembrane protein with tyrosine kinase activity a member of the HER receptor family and is involved in signal transduction pathways, leading to cell growth and differentiation. In gastric and gastroesophageal cancer, the frequency of HER2 overexpression varies widely in the literature. However, HER2 testing in gastric cancer differs from testing in breast cancer because of inherent differences in tumor biology, intratumoral heterogeneity of HER2 expression and incomplete membrane staining that are commonly observed in gastric tumors.

HER2 status is typically assessed by immunohistochemistry (IHC) or in situ hybridization (ISH) assays. Both methods can be practices using formalin-fixed and paraffin-embedded biopsy tissues or surgical specimens and occasionally, cytological samples. Fluorescent in situ hybridization (FISH) is regarded to be the gold standard and allows counting of number of copies of the HER2 gene. However, FISH has a higher cost, and as a result IHC is also used widely, with 3+ scores by IHC being considered as HER2 overexpressing and equivocal (2+ score) cases by IHC being further subjected to FISH.

Assays for assessing tumor cell expression of HER2 are well-known in the art. Examples of FISH assays include HER2 IQFISH pharmDx (Agilent) or HER2 FISH pharmDx™ (Dako), the PathVysion™ HER-2 DNA Probe Kit II (Abbott) which are designed to detect amplification of the HER-2/neu gene in formalin-fixed, paraffin-embedded. Exemplary assays also include the FDA-approved SPOT-Light HER2 CISH assay. Chromogenic in situ hybridization (CISH) detects HER2 gene amplification. This technique, also referred to as Subtraction Probe Technology Chromogenic In Situ Hybridization, is a test used see if breast cancer cells overexpress HER2 receptor proteins at the cell surface. Another widely used assay for HER2 is the HercepTest™ (Dako North America, Inc.), a semiquantitative immunohistochemical assay used to determine HER2 protein overexpression in formalin-fixed, paraffin-embedded cancer tissue. For example, tumors expressing low levels of HER2 can be identified by a score of +1 to +2 via HercepTest™.

In one embodiment, an individual treated according to the disclosure has a gastric cancer (e.g., a stomach cancer or a gastroesophageal or gastroesophageal junction cancer) that tests positive for HER2 protein, optionally wherein the cancer overexpresses HER2 protein (particularly as a result of HER2 gene amplification), for example the tumor is be characterized by a 3+ score in IHC and/or is positive by FISH for gene amplification. In certain optional embodiments, the tumor is characterized by low levels of HER2 protein (lower than excess HER2 expression), for example the tumor may be characterized by a 2+ score in IHC and is negative by FISH for gene amplification. In one embodiment, the individual is treated with an antibody that neutralizes the 5′-ectonucleotidase activity of human CD73 protein, in combination with an antibody that binds HER2 polypeptides (e.g., an antibody comprising the heavy and light chain CDRs or variable regions of trastuzumab), optionally further in combination with a chemotherapy agent.

In some embodiments, provided is a method of treating a tumor in an individual having a gastric cancer, comprising: (a) assessing HER2 expression by a tumor (or tumor cells) in the individual; and (b) if the individual has a tumor (or tumor cells) characterized by HER2 expression, optionally HER2 overexpression, administering to the individual an effective amount of an antibody that neutralizes the 5′-ectonucleotidase activity of human CD73 protein, optionally in combination with an effective amount of an antibody that binds HER2 polypeptides (e.g., an antibody comprising the heavy and light chain CDRs or variable regions of trastuzumab), optionally further in combination with a chemotherapy agent.

In one embodiment, a tumor characterized by HER2 expression or HER2 overexpression, is characterized by a positive result for HER2 gene amplification, in a FISH assay. In one embodiment, a tumor is characterized by a 2+ or 3+ score by IHC. In one embodiment, a tumor is characterized by a 3+ score by IHC. In one embodiment, a HER2 overexpressing tumor is a tumor characterized by a 3+ score by IHC. In one embodiment, HER2 expression or HER2 overexpression is determined according to the ToGA FISH scoring scheme for HER2 testing in gastric and gastroesophageal junction cancer (see van Cutsem et al. Gastric Cancer. 2015; 18(3): 476-484). In one example, HER2 overexpression is characterized by a HER2 gene copy number of at least 6 signals/nucleus. In one example, HER2 overexpression is characterized by a HER2 gene copy number of greater than 6 signals/nucleus. In another example, the ratio of HER2 signals to chromosome 17 centromere signals is determined. For example for FISH amplification, a DNA probe kit can use a dual-color probe for determining the number of copies of HER2 (orange) and chromosome 17 centromeres (green). In one example, HER2 overexpression is characterized by a ratio of HER2 signals to chromosome 17 centromere of at least 2, optionally at least 2.2, or optionally greater than 2.2.

In one embodiment, the antibody that binds HER2 used in the combination treatments of the disclosure is an antibody of human isotype (e.g. human IgG1) which is not conjugated to a cytotoxic agent (e.g., the antibody is a “naked” antibody). Optionally, the method further comprises administration to the individual a therapeutically effective amount of a chemotherapy agent.

In another embodiment, the antibody that binds HER2 used in the combination treatments of the disclosure is an antibody drug conjugate (ADC). For example, the antibody that binds HER2 can be conjugated to a cytotoxic agent, optionally an auristatin, a maytansinoid (e.g. DM1) or a camptothecin (e.g. camptothecin or exatecan derivatives, DXd, or SN38); optionally wherein the ADC is trastuzumab emtansine or trastuzumab deruxtecan (DS-8201a).

Combined administration can include simultaneous administration of the compounds in different dosage forms, or separate administration of the compounds (e.g., sequential administration). Thus, a CD73-binding antibody and a HER2-binding antibody (and optionally further a chemotherapy agent) can be used in combination. The anti-CD73 antibody and the HER2-binding antibody can be formulated for separate administration and administered concurrently or sequentially. The CD73-binding antibody and a HER2-binding antibody can also be used in combination with a chemotherapy agent, for example a topoisomerase inhibitor, a taxane, paclitaxel. The anti-CD73 antibody, HER2-binding antibody and/or chemotherapy agent can each be formulated for separate administration and administered concurrently or sequentially, or when the HER2-binding antibody is conjugated (e.g. covalently bound) to the chemotherapy agent, the anti-CD73 antibody and the HER2-binding ADC can each be formulated for separate administration and administered concurrently or sequentially.

In some embodiments, a combination treatment of the disclosure comprises at least one course of treatment, wherein for each course of treatment, a plurality (e.g., two, three or four) of successive doses of the anti-CD73 antibody are administered and a plurality (e.g., two, three or four) of successive doses of anti-HER2 antibody are administered. Optionally, the treatment regimen further comprises administration of a plurality (e.g., two, three or four) of successive doses of chemotherapy agent (e.g. a taxane).

In some embodiments, a combination treatment of the disclosure comprises:
(a) a plurality of administrations (e.g. at least two, three or four) of a dose of the anti-CD73 antibody, optionally wherein the anti-CD73 antibody is administered once every two or three weeks; and
(b) a plurality of administrations (e.g. at least two, three or four) of a dose of the anti-HER2 antibody, optionally wherein the anti-HER2 antibody is administered once every week or once every three weeks. In one administration regimen, the anti-CD73 antibody is administered once every two weeks and the anti-HER2 antibody is administered once every three weeks. In another administration regimen, the anti-CD73 antibody is administered once every three weeks and the anti-HER2 antibody is administered once every three weeks, wherein the anti-CD73 antibody and anti-HER2 antibody are administered on the same day.

In some embodiments, a combination treatment of the disclosure comprises:
(a) a plurality of administrations (e.g. at least two, three or four) of a dose of the anti-CD73, optionally wherein the anti-CD73 antibody is administered once every two or three weeks; and
(b) a plurality of administrations (e.g. at least two, three or four) of a dose of chemotherapy agent (e.g. a platinum agent, a topoisomerase inhibitor, a taxane, a camptothecin), optionally wherein the chemotherapy agent is administered weekly or once every three weeks. In one exemplary administration regimen, the anti-CD73 antibody is administered once every three weeks and the chemotherapy agent is administered once every three weeks, wherein the anti-CD73 antibody and chemotherapy agent are administered on the same day.

In some embodiments, a combination treatment of the disclosure comprises:
(a) a plurality of administrations (e.g. at least two, three or four) of a dose of the anti-CD73, optionally wherein the anti-CD73 antibody is administered once every two weeks or once every three weeks;
(b) a plurality of administrations (e.g. at least two, three or four) of a dose of the anti-HER2 antibody are administered, optionally wherein the anti-HER2 antibody is administered once every week or once every three weeks; and
(c) a plurality of administrations (e.g. at least two, three or four) of a dose of chemotherapy agent (e.g. a topoisomerase inhibitor, a taxane, a camptothecin), optionally wherein the chemotherapy agent is administered weekly or once every three weeks.

In some embodiments, where the anti-HER2 antibody is conjugated to the chemotherapy agent, a combination treatment regimen the disclosure can comprise:
(a) a plurality of administrations (e.g. at least two, three or four) of a dose of the anti-CD73, optionally wherein the anti-CD73 antibody is administered once every two weeks or once every three weeks;
(b) a plurality of administrations (e.g. at least two, three or four) of a dose of the anti-HER2 antibody chemotherapy drug conjugate are administered, optionally wherein the anti-HER2 antibody chemotherapy drug conjugate is administered once every three weeks.

In any of the embodiments, a treatment of the disclosure comprises at least 4, 5 or 6 administrations of the anti-CD73 antibody. In any of the embodiments, treatment according to the regimen of the disclosure comprises at least 4, 5 or 6 administrations of the anti-HER2 antibody. In any of the embodiments, a treatment according to the disclosure comprises at least 4, 5 or 6 administrations of the chemotherapy agent. In any of the embodiments, treatment according to the regimen of the disclosure comprises at least 6 administration of the anti-CD73 antibody, at least 4 administrations of the anti-HER2 antibody, and optionally further at least 4 administrations of the chemotherapy agent.

In any of the embodiments, the first administration of anti-CD73 antibody takes place before the first administration of the anti-HER2 antibody and/or chemotherapy agent. For example, the first administration of anti-CD73 antibody can take place one week or two weeks before the first administration of the anti-HER2 antibody and/or chemotherapy agent.

In any of the embodiments, where anti-CD73 antibody and anti-HER2 antibody are administered on the same day, optionally administration of anti-CD73 antibody takes place before (e.g., 1-12 hours before) administration of the anti-HER2 antibody. In any of the embodiments, where anti-CD73 antibody and chemotherapy agent are administered on the same day, optionally administration of anti-CD73 antibody takes place before (e.g., 1-12 hours before) administration of the chemotherapy agent.

In the treatment methods, the anti-HER2 antibody and the anti-CD73 antibody can be administered separately, together or sequentially, or in a cocktail. In some embodiments, the anti-CD73 antibody is administered prior to the anti-HER2 antibody. For example, the anti-CD73 antibody can be administered approximately 1 hour to 7 or 8 days, prior to the administration of the anti-HER2 antibody. In some embodiments, an anti-CD73 antibody is administered from about 30 minutes to about 1 week, from about 1 hour to about 1 week, from about 1 hour to about 2 hours, from about 1 hour to about 6 hours, from about 1 hour to about 12 hours, from about 2 hours to about 4 hours, from about 4 hours to about 6 hours, from about 6 hours to about 8 hours, from about 8 hours to 1 day, or from about 1 to 8 days prior to the administration of the anti-HER2 antibody. In some embodiments, an anti-CD73 antibody is administered concurrently with the administration of the anti-HER2 antibody.

In any of the embodiments, an administration of anti-HER2 antibody and/or chemotherapy agent occurs no more than about 1 week after an administration of anti-CD73 antibody. For example, where a method comprises a plurality of administrations of a dose of anti-HER2 antibody and a plurality of administrations of a dose of anti-CD73 antibody, each administration of anti-HER2 antibody can occur either on the same day or about 1 week (e.g. 7 days, 8 days) after an administration of anti-CD73 antibody.

In any of the embodiments, each of a plurality of administrations of anti-HER2 antibody and each administration of chemotherapy agent occurs no more than 14 days or no more than about 2 weeks after, optionally 7 days or about 1 week after, an administration of anti-CD73 antibody. In any of the embodiments, each of a plurality of administrations of anti-HER2 antibody and each of a plurality of administrations of chemotherapy agent occurs within 14 days or about 2 weeks after, optionally 7 days or about 1 week after, an administration of anti-CD73 antibody.

Optionally, a treatment is configured such that the first administration of anti-HER2 antibody and/or a first administration of chemotherapy agent occurs within 14 days or about 2 weeks after an administration of anti-CD73 antibody, and wherein each subsequent (i.e. second or further) administration of anti-HER2 antibody and/or each subsequent administration of chemotherapy agent are either administered on the same day as an administration of anti-CD73 antibody or are preceded by an administration of anti-CD73 antibody by no more than 7 days (e.g. 1 week).

Accordingly, in any of the embodiments, each subsequent (i.e. second or further) administration of anti-HER2 antibody and/or each administration of chemotherapy agent is either administered on the same day as an administration of anti-CD73 antibody or are preceded by an administration of anti-CD73 antibody by no more than 8 days, or no more than 8 days (e.g. about 1 week).

In any of the embodiments, each subsequent (i.e. second or further) administration of anti-HER2 antibody occurs either on the same day or within 7 or 8 days or within about 1 week after an administration of anti-CD73 antibody. In any of the embodiments, each administration of anti-HER2 antibody occurs either on the same day or no more than 7 or 8 days or about 1 week after an administration of anti-CD73 antibody.

In any of the embodiments, each subsequent (i.e. second or further) administration of chemotherapy agent occurs either on the same day or within 7 or 8 days or within about 1 week after an administration of anti-CD73 antibody. In any of the embodiments, each administration of chemotherapy agent occurs either on the same day or no more than 7 or 8 days or about 1 week after an administration of anti-CD73 antibody.

Anti-CD73 antibody can thus for example be administered every two weeks, and anti-HER2 antibody (and chemotherapy agent, when used) can be administered every three weeks. An exemplary therapeutic regimen comprises administration of anti-CD73 antibody every two weeks, administration of anti-HER2 antibody (and chemotherapy agent, when used) every three weeks, wherein the first administration of anti-HER2 antibody (and chemotherapy agent, when used) occurs about two weeks, or within about two weeks, e.g. within 15 days, after the first administration of anti-CD73 antibody.

In any embodiment, the individual can be characterized as having a cancer which has progressed or relapsed following a prior therapy, or which has not responded to a prior therapy, optionally further wherein the prior therapy comprises administration of a chemotherapy agent, optionally a platinum agent (e.g. cisplatin) or a taxane agent (e.g. paclitaxel).

In any embodiment, the individual can be characterized as having a cancer which has progressed, relapsed or not responded to prior treatment with an antibody that binds HER2 polypeptides (e.g., an antibody comprising the heavy and light chain CDRs, variable regions, or polypeptide chains of trastuzumab).

In another example, the anti-CD73 antibody can be administered in an amount that achieves or maintains, for a specified period of time (e.g. at least one week, two weeks, three weeks), a concentration in circulation, optionally in an extravascular tissue of interest (e.g., the tumor or tumor environment), that is higher than the EC₅₀, optionally EC₇₀ or optionally EC₉₀, for binding to CD73-expressing cells (e.g., as assessed by titrating anti-CD73 antibody on CD73-expressing cells, for example MDA-MB-231 cells). Optionally the concentration achieved is at least 20%, 50% or 100% higher than the EC₅₀, optionally EC₇₀ or optionally EC₉₀, for binding to CD73-expressing cells.

In another example, the anti-CD73 antibody can be administered in an amount effective to achieve and/or maintain for at least one week, optionally at least two or three weeks and/or until the subsequent administration of anti-CD73 antibody) in an individual a blood concentration of at least the EC₅₀, optionally the EC₇₀, optionally substantially the EC₉₀, for inhibition of CD73-mediated catabolismof AMP to adenosine (e.g., by assessing neutralization of 5′ ectonucleotidase activity in MDA-MB-231 or A375 cells by quantifying hydrolysis of AMP to adenosine). In one embodiment, the amount of anti-CD73 antibody is an amount effective to achieve (or maintain, optionally for at least one week, optionally two weeks) the EC₅₀, optionally the EC₇₀, optionally substantially the EC₉₀, for inhibition of CD73-mediated catabolismof AMP to adenosine in an extravascular tissue of an individual.

Anti-CD73 antibodies are further described herein. For example, the antibody having the heavy and light chain CDRs of an antibody of the disclosure, for example an antibody having the respective heavy and light chain amino acid sequences of 47 and 48, or a function-conservative variant thereof, can be administered in a dose of 900-3000 mg, optionally the antibody is administered at a dose of at least 900 mg, optionally 1500 mg or at least 1500 mg, or optionally 2400 mg or at least 2400 mg. The antibody can be administered intravenously.

In another example, the antibody having the heavy and light chain CDRs the heavy and light chain variable regions of SEQ ID NOS: 42 and 43 can be administered in a dose of at least 900 mg, optionally 1500 mg or at least 1500 mg, or optionally 2400 mg or at least 2400 mg. The antibody can be administered intravenously.

The chemotherapy agent, when not conjugated to an anti-HER2 antibody, can be administered in a suitable dose for the particular agent. For example, a taxane agent can be administered. Taxanes are chemotherapeutic agents including paclitaxel and docetaxel that produce antitumor activity by causing stabilization of cellular microtubules, thereby inhibiting cell division. For example, paclitaxel can be administered every 3 weeks in a dose of 175 mg/m², body surface area via a 3 hour infusion. In one optional embodiment, the combination treatments of the disclosure comprise treatment with a chemotherapy agent (e.g. a taxane, a chemotherapy agent conjugated to an anti-HER antibody), in the absence of (treatment with) a platinum-based agent (e.g. cisplatin, oxaliplatin, carboplatin). In one optional embodiment, the combination treatments of the disclosure comprise treatment with a chemotherapy agent (e.g. a taxane, a chemotherapy agent conjugated to an anti-HER antibody), in the absence of (treatment with) an anthracycline derivative (e.g. doxorubicin, daunorubicin, epirubicin).

In any embodiment herein, a treatment of the disclosure can be specified as being in the absence of combined treatment with an antibody that neutralizes PD-1. The term “neutralizes PD-1” refers to a process in which PD-1 is inhibited in its signal transduction capacity resulting from the interaction of PD-1 with one or more of its binding partners, such as PD-L1 or PD-L2. An antibody that neutralizes PD-1 can bind to a human PD-1 protein or to a PD-L1 protein, for example. Examples include pembrolizumab (trade name Keytruda®), atezolizumab (trade name Tecentriq™), durvalumab (trade name Imfinzi™).

Several antibodies that bind specifically to human HER2 (anti-HER2) antibodies have been developed, including notably trastuzumab. Other molecular HER2-targeted antibodies have been tested or are currently being tested such as pertuzumab, margetuximab, and the antibody-drug conjugates trastuzumab-emtansine and trastuzumab-deruxtecan.

In certain embodiments, particularly for the treatment of gastric cancers, the anti-HER2 antibody can for example be administered intravenously or subcutaneously every three weeks, with (in the case of intravenous administration) or without an additional loading dose administration preceding the subsequent successive treatment doses. In any of the embodiments, optionally, the first administration of anti-HER2 antibody can be a loading dose, followed one week later by administration of subsequent treatment doses anti-HER2 antibody, wherein the subsequent treatment doses are administered once every three weeks. For example on day 1 (start of week 1) the individual can be treated with anti-CD73 antibody; on day 15 (start of week 3) the individual can be treated with anti-CD73 antibody, a loading dose of anti-HER2 antibody and a chemotherapy agent; on day 21 the individual can be treated with a treatment dose of anti-HER2 antibody; wherein subsequent doses of the anti-CD73 antibody are administered every two or three weeks after the preceding administration of the anti-CD73 antibody, wherein subsequent doses of the treatment doses anti-HER2 antibody are administered every three weeks after the preceding administration of treatment dose of the anti-HER2 antibody, and wherein subsequent doses of the chemotherapy agent are administered every three weeks after the preceding administration of the chemotherapy agent.

Anti-HER2 antibodies are further described herein. For example, an antibody can comprise the respective heavy and light chain CDRs, variable regions or full polypeptide chains of trastuzumab, administered at a dose of 2-8 mg/kg body weight (including loading doses), optionally 2-6 mg/kg body weight (for treatment doses). When present as a naked antibody of human IgG1 isotype (non-conjugated to a cytotoxic agent) such as the antibody composition approved as Herceptin™, such antibody can be administered as a loading dose (first administration) of 8 mg/kg body weight via a 90 minute infusion, followed one week later by treatment doses of 6 mg/kg body weight administered every 3 weeks via a 30 minute infusion. Trastuzumab, approved as Herceptin™, can also be used by weekly administration (particular for breast cancer), for example in an initial dose of 4 mg/kg as a 90 minute IV infusion followed by subsequent weekly doses of 2 mg/kg as 30 minute IV infusions. Trastuzumab has also been approved as Herceptin™MSC, a formulation suitable for subcutaneous administration, and containing the novel excipient (carrier for active ingredients of a medication), rHuPH20. rHuPH20 (recombinant human hyaluronidase) reversibly breaks down a gel-like substance (hyaluronan) that forms a barrier in the tissues between cells under the skin. Herceptin™MSC is administered every three weeks as a fixed dose of 600 mg in a total fix volume of 5 ml, without loading dose, over a period of up to 5 minutes.

In another example, an antibody comprising the heavy and light chain CDRs, variable regions or full polypeptide chains of trastuzumab is conjugated to a cytotoxic agent, thereby combining the anti-HER2 and chemotherapy agent in a single molecule or composition. Such an ADC, for example the ADC trastuzumab deruxtecan (DS-8201; fam-trastuzumab deruxtecan-nxki; Enhertu®, Daiichi Sankyo) can be administered at a dose of 5.4 mg/kg body weight administered every 3 weeks via intravenous infusion. The ADC trastuzumab emtansine (ado- trastuzumab emtansine; Kadcyla®, Genentech) can be administered at a dose of 3.6 mg/kg body weight administered every 3 weeks via intravenous infusion.

The treatment regimens of the disclosure can be advantageously used to treat a HER2-positive gastric gastroesophageal junction adenocarcinoma characterized by the presence of CD73-expressing cells in the tumor stromal tissue. A CD73-positive cancer can thus be characterized by presence of CD73-expressing cells in the tumor or tumor environment. As shown herein, HER2-expressing tumors are characterized by CD73 expression in the tumor stroma. Accordingly, an individual having a gastric or gastroesophageal junction adenocarcinoma can be treated with the treatment regimens of the disclosure with our without a prior detection step to assess expression of CD73 on cells in the tumor microenvironment (e.g. on endothelial cells and/or immune cells, for example B cells, CD8 T cells, naïve CD8 T cells).

Optionally, a treatment method can comprise a step of detecting a CD73 nucleic acid or polypeptide in a biological sample of a tumor from an individual (e.g., in cancer tissue, tissue proximal to or at the periphery of a cancer, cancer adjacent tissue, adjacent non-tumorous tissue or normal adjacent tissue). A determination that a biological sample is characterized by CD73 polypeptides, e.g. comprises cells expressing CD73, can indicate that the patient has a cancer that may have a strong benefit from treatment an anti-CD73 antibody and an anti-HER2 antibody (and optionally further a chemotherapy agent). A patient having a cancer suitable for treatment according to the disclosure may be determined to have tumor stroma characterized by cells that express at their surface CD73, that express CD73 at a high level (e.g. compared to a reference value, compared to a healthy individual, at a level corresponding to individuals that are poor responders for treatment with an anti-CD73 agent), or that show high intensity of staining with an anti-CD73 antibody (e.g., as determined by IHC).

In one embodiment, the method comprises determining the level of expression of a CD73 nucleic acid or polypeptide in a biological sample (e.g. comprising tumor and/or tumor stromal tissue) and comparing the level to a reference level corresponding to a healthy individual. A determination that the biological sample comprises cells expressing CD73 nucleic acid or polypeptide at a level that is increased compared to the reference level indicates that the patient has a cancer that can be advantageously treated with (e.g. can derive particular benefit from) an anti-CD73 antibody and an anti-HER2 antibody (and optionally further a chemotherapy agent). Optionally, detecting a CD73 polypeptide in a biological sample comprises detecting CD73 polypeptide expressed on the surface of a malignant cell, an endothelial cell, a T cell and/or a B cell. In one example, CD73 polypeptides will be present on at least 10%, 20% 30%, 40%, 50%, 60%, 70%, 80%, or more of the cells in tumor tissue or tumor-adjacent tissue sample (e.g. biopsy) taken from an individual.

Determining whether an individual has a cancer characterized by cells that express a CD73 polypeptide can for example comprise obtaining a biological sample (e.g. by performing a biopsy) from the individual that comprises cells from the cancer environment (e.g. tumor or tumor adjacent tissue), bringing said cells into contact with an antibody that specifically binds a human CD73 polypeptide, and detecting whether the cells express CD73 on their surface. Optionally, determining whether an individual has cells that express CD73 comprises conducting an immunohistochemistry assay.

In one embodiment, the disclosure provides a method for the treatment or prevention of a HER2-positive gastric cancer in an individual in need thereof (e.g., in a subject having a gastric or gastroesophageal junction adenocarcinoma), the method comprising:
a) detecting CD73 polypeptide (e.g. CD73-expressing cells) in a tumor tissue sample from the individual, optionally comprising tumor and/or tumor-adjacent tissue, and
b) upon a determination that tumor tissue sample comprises CD73 polypeptides (e.g. CD73-expressing cells), optionally at a level that is increased compared to a reference level, administering to the individual an anti-CD73 antibody and an anti-HER2 antibody (and optionally further a chemotherapy agent). Optionally, detecting CD73 polypeptide or CD73-expressing cells in a tumor tissue sample comprises obtaining from the individual a biological sample that comprises tumor tissue and/or tissue proximal to or at the periphery of a tumor (e.g., tumor adjacent tissue, adjacent non-tumorous tissue or normal adjacent tissue), and detecting levels of CD73 polypeptide or CD73-expressing cells. CD73-expressing cells may comprise, for example, endothelial cells, tumor cells, CD4 T cells, CD8 T cells, B cells.

In one embodiment, both HER2 and CD73 expression can be assessed. For example, in one embodiment, the disclosure provides a method for the treatment or prevention of a gastric cancer in an individual in need thereof (e.g., in a subject having a gastric or gastroesophageal junction adenocarcinoma), the method comprising:
a) detecting CD73 polypeptide (e.g. CD73-expressing cells) in the tumor environment, optionally within a tumor and/or within tumor-adjacent tissue,
b) detecting HER2 expression (e.g. expression levels) on tumor cells, and c) upon a determination that tumor environment comprises CD73 (e.g., CD73-expressing cells), optionally at a level that is increased compared to a reference level, and upon a detection of HER2-expression (e.g. overexpression) on tumor cells, administering to the individual an anti-CD73 antibody and an anti-HER2 antibody (and optionally further a chemotherapy agent). Optionally, detecting CD73 polypeptide or CD73-expressing cells within the tumor environment comprises obtaining from the individual a biological sample that comprises tumor tissue and/or tissue proximal to or at the periphery of a cancer (e.g., tumor adjacent tissue, adjacent non-tumorous tissue or normal adjacent tissue), and detecting levels of CD73 polypeptide or CD73-expressing cells. CD73-expressing cells may comprise, for example, endothelial cells, tumor cells, CD4 T cells, CD8 T cells, B cells. Optionally, detecting (or detection of) HER2-expression (e.g. overexpression) on tumor cells comprises detecting (or detection of) HER2 overexpression in tumor cells. Optionally, detecting HER2-expressing or HER2 overexpressing tumor cells comprises obtaining from the individual a biological sample that comprises tumor tissue, and detecting expression levels of HER2 polypeptide, optionally as determined by FISH (e.g. presence of increased HER2 gene copy number) and/or by IHC (e.g. a 3+ score in an IHC assay).

In one embodiment, the treatment regimens of the disclosure can advantageously be used treat an individual (or a population of individuals) who has a HER2-positive cancer and who has received a previous course of treatment with a chemotherapy agent (e.g. a platinum-based agent, carboplatin, a taxane agent, paclitaxel). Optionally, the previous course of treatment with a chemotherapy agent has been completed within the preceding 1, 2, 3, 4, 5, or 6 months. In one embodiment, in one aspect the treatment regimens of the disclosure can be specified as being in the absence of a step of prior testing of CD73 expression in cells (e.g. in a biopsy; in cells from tumor stroma, on tumor cells). In one embodiment, provided is a method for the treatment or prevention of a gastric cancer in an individual (or a population of individuals) who has (have) received a previous course of treatment with a chemotherapy agent (e.g. a platinum agent, carboplatin, a taxane agent, paclitaxel), the method comprising:
a) detecting HER2-expressing tumor cells, optionally within a tumor and/or within adjacent tissue, and
b) upon a detection of HER2-expressing (e.g. overexpressing) tumor cells, administering to the individual an anti-CD73 antibody and an anti-HER2 antibody (and optionally further a chemotherapy agent). Optionally, detecting HER2-expressing or HER2 overexpressing tumor cells comprises obtaining from the individual a biological sample that comprises cancer tissue, and detecting expression levels of HER2 polypeptide, optionally as determined by FISH (e.g. presence of increased HER2 gene copy number) and/or by IHC (e.g. a 3+ score in an IHC assay).

The efficacy and/or therapeutically effective amount of the treatment can be measured by various endpoints commonly used in evaluating cancer treatments. Exemplary endpoints include: extending survival (including OS and PFS); resulting in an objective response (including a CR or a PR); tumor regression, tumor weight or size shrinkage, longer time to disease progression, increased duration of survival, longer PFS, improved OS rate, increased duration of response, and improved quality of life and/or improving signs or symptoms of cancer. The improvements and/or efficacy can be compared to other treatments, for example to treatment comprising administration of anti-HER2 antibodies and/or chemotherapy in the absence of the anti-CD73 antibodies.

As used herein, the term “progressive disease” (PD) refers to least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. The appearance of one or more new lesions is also considered progression.

As used herein, the term “partial response,” (PR) refers to at least a 30% decrease in the sum of diameters of target lesions, taking as reference the baseline sum diameters.

As used herein, the term “complete response” (CR) refers to the disappearance of all target lesions with the short axes of any target lymph nodes reduced to <10 mm.

As used herein, the term “stable disease” (SD) refers to neither sufficient shrinkage to qualify for PR nor sufficient increase to qualify for PD, taking as reference the smallest sum of diameters while on study.

As used herein, the term “objective response rate” (ORR) is equal to the proportion of patients achieving a best overall response of partial or complete response (PR+CR) according to RECIST 1.1.

As used herein, the term “overall survival” (OS) refers to the percentage of patients remaining alive for a defined period of time, such as 1 year, 5 years, etc. from the time of diagnosis or treatment. In a preferred embodiment, OS refers to the time from the date of randomization in the Study to the date of death from any cause. If the patient is alive at the end of the follow-up period or is lost to follow-up, OS data is censored on the last date the patient is known to be alive. Overall survival is evaluated by the Kaplan-Meier method, and a 95% confidence interval (CI) is provided for the median OS in each treatment arm.

As used herein, the term “progression-free survival” (PFS) refers to the patient remaining alive without the cancer progressing or getting worse. In a preferred aspect of the invention, PFS is defined as the time from randomization in the Study until the first radiographic documentation of objective progression as defined by RECIST (Version 1.1), or death from any cause. Patients who die without a reported prior progression will be considered to have progressed on the day of their death. Patients who did not progress or are lost to follow-up will be censored at the day of their last radiographic tumor assessment.

As used herein, the term “disease control rate” (DCR) refers to lack of disease progression and rate thereof. It refers to the group of patients with a best overall response categorized as CR, PR or SD (specifically excluding the patients with PD), wherein the best overall response is the best response recorded from the start of treatment until PD.

As used herein, the term “clinical benefit rate,” refers to SD or better at the time of analysis. The tumor response rate of SD or better (i.e. CR+PR+SD) is defined as the proportion of patients with a response of SD or better, as defined by RECIST 1.1.

As used herein, the term “extending survival” is meant as increasing OS or PFS in a treated patient relative to i) an untreated patient, ii) a patient treated with less than all of the anti-tumor agents in a particular combination therapy, or iii) a control treatment protocol. Survival is monitored following the initiation of treatment or following the initial diagnosis of cancer.

As used herein, the term “best overall response” is the best response recorded from the start of the study treatment until the earliest of objective progression or start of new anticancer therapy, taking into account any requirement for confirmation. The patient's best overall response assignment will depend on the findings of both target and non-target disease and will also take into consideration the appearance of new lesions. The best overall response will be calculated via an algorithm using the assessment responses provided by the investigator over the course of the trial.

An exemplary method of treating a cancer (e.g., an advanced gastric or gastroesophageal junction adenocarcinoma) comprises administering to a patient in need thereof, an effective amount of an anti-human HER2 antibody comprising a light chain variable region having the amino acid sequence of SEQ ID NO: 24 and a heavy chain variable region having the amino acid sequence of SEQ ID NO: 23 in simultaneous, separate, or sequential combination with an effective amount of an anti-human CD73 antibody comprising a light chain variable region having the amino acid sequence of SEQ ID NO: 43 and a heavy chain variable region having the amino acid sequence of SEQ ID NO: 42; optionally wherein the anti-human HER2 antibody comprises a light chain having the amino acid sequence of SEQ ID NO: 26 and a heavy chain having the amino acid sequence of SEQ ID NO: 25; optionally, wherein the anti-human CD73 antibody comprises a light chain having the amino acid sequence of SEQ ID NO: 48 and a heavy chain having the amino acid sequence of SEQ ID NO: 47.

### Antibodies That Bind HER2

Binding HER2 and blocking HER2 signaling and limiting the number of available membrane molecules of HER2, has been the focus of several therapeutic approaches. Trastuzumab (Herceptin®, Roche) is a humanized version of murine 4D5 antibody which binds to the juxtamembrane region of HER2. Trastuzumab exerts its antiproliferative activity by mediating antibody-dependent cellular cytotoxicity (ADCC) toward tumor cells to which it binds, by causing HER2 internalization and degradation, and by inhibiting MAPK and PI3K/Akt pathways via inhibition of HER2 dimerization. Trastuzumab comprises the heavy and light chain variable regions shown below and is produced as a human IgG1 isotype. Trastuzumab full heavy and light chain polypeptide chains shown below (SEQ ID NOS: 25 and 26). Another example of an anti-HER2 antibody is margetuximab which binds the same epitope on HER2 that is bound by trastuzumab. Margetuximab has the heavy and light chain amino acid sequences shown in SEQ ID NOS: 27 and 28 (Kabat CDRs underlined).

More recently, trastuzumab conjugated to different chemotherapy agents have been developed. These immunoconjugates or ADCs include notably trastuzumab emtansine (also known as PRO132365, RG3502, T-DM1, trastuzumab-MCC-DM1, Kadcyla®), trastuzumab deruxtecan (also known as DS-8201, DS-8201a, fam-trastuzumab deruxtecan-nxki, Enhertu®), and trastuzumab duocarmazine (also known as SYD985, trastuzumab vc-seco-DUBA, trastuzumab valine-citrulline-seco-duocarmycinhydroxybenzamide-azaindole). Trastuzumab deruxtecan, trastuzumab duocarmazine and trastuzumab emtansine share the same heavy and light variable regions and full polypeptide chains of trastuzumab, with the proviso that trastuzumab emtansine lacks terminal lysine on the heavy chain of trastuzumab. Trastuzumab deruxtecan (DS8201a) is conjugated with deruxtecan, a camptothecin or exatecan derivative referred to as DXd (DX-8951 derivative, DXd) via a linker on an average of 8 cysteinyl residues (see Ogitano et al. (2016) Cancer Sci 107 (2016) 1039-1046, the disclosure of which is incorporated herein by reference). Trastuzumab deruxtecan can be prepared as described in U.S. Pat. No. 10,195,288 and PCT publication No. WO2019/044946, the disclosures of which are incorporated herein by reference. Trastuzumab emtansine is conjugated with maytansinoid DM1 via a noncleavable succinimidyl-4-(N-maleimidomethyl) cyclohexane-1-carboxylate (SMCC) linker on an average of 3-4 lysyl residues. Trastuzumab duocarmazine is conjugated with seco-DUBA via a cleavable linker N-[2-(2 maleimidoethoxy)ethoxycarbonyl]-L-valyl-L-citrullinyl-p-aminobenzyloxycarbonyl-N-[2-(2- hydroxyethoxy)ethyl]-N-[2-(methylamino)ethyl]carbamoyl on an average of 2 or 4 cysteinyl residues.

### Trastuzumab Heavy Chain Variable Region

| (SEQ ID NO: 23) | |
|---|---|
| EVQLVESGGG LVQPGGSLRL SCAASGFNIK DTYIHWVRQA PGKGLEWAVR | 50 |
| IYPTNGYTRY ADSVKGRFTI SADTSKNTAY LQMNSLRAED TAVYYCSRWG | 100 |
| GDGFYAMDYW GQGTLVTVSS. | |

### Trastuzumab Light Chain Variable Region

| (SEQ ID NO: 24) | |
|---|---|
| DIQMTQSPSS LSASVGDRVT ITCRASQDVN TAVAWYQQKP GKAPKLLIYS | 50 |
| ASFLYSGVPS RESGSRSGTD FTLTISSLQP EDFATYYCQQ HYTTPPTFGQ | 100 |
| GTKVEIK | |

### Trastuzumab Heavy Chain

| (SEQ ID NO: 25) | |
|---|---|
| EVQLVESGGG LVQPGGSLRL SCAASGENIK DTYIHWVRQA PGKGLEWVAR | 50 |
| IYPTNGYTRY ADSVKGRFTI SADTSKNTAY LQMNSLRAED TAVYYCSRWG | 100 |
| GDGFYAMDYW GQGTLVTVSS ASTKGPSVFP LAPSSKSTSG GTAALGCLVK | 150 |
| DYFPEPVTVS WNSGALTSGV HTFPAVLQSS GLYSLSSVVT VPSSSLGTQT | 200 |
| YICNVNHKPS NTKVDKKVEP KSCDKTHTCP PCPAPELLGG PSVELFPPKP | 250 |
| KDTLMISRTP EVTCVVVDVS HEDPEVKENW YVDGVEVHNA KTKPREEQYN | 300 |
| STYRVVSVLT VLHQDWLNGK EYKCKVSNKA LPAPIEKTIS KAKGQPREPQ | 350 |
| VYTLPPSREE MTKNQVSLTC LVKGFYPSDI AVEWESNGQP ENNYKTTPPV | 400 |
| LDSDGSFFLY SKLTVDKSRW QQGNVFSCSV MHEALHNHYT QKSLSLSPGK. | 450 |

### Trastuzumab Light Chain

| (SEQ ID NO: 26) | |
|---|---|
| DIQMTQSPSS LSASVGDRVT ITCRASQDVN TAVAWYQQKP GKAPKLLIYS | 50 |
| ASFLYSGVPS RESGSRSGTD FTLTISSLQP EDFATYYCQQ HYTTPPTFGQ | 100 |
| GTKVEIKRTV AAPSVFIFPP SDEQLKSGTA SVVCLLNNFY PREAKVQWKV | 150 |
| DNALQSGNSQ ESVTEQDSKD STYSLSSTLT LSKADYEKHK VYACEVTHQG | 200 |
| LSSPVTKSEN RGEC. | 214 |

### Margetuximab Heavy Chain

| (SEQ ID NO: 27) | |
|---|---|
| QVQLQQSGPE LVKPGASLKL SCTASGENIK DTYIHWVKQR PEQGLEWIGR | 50 |
| IYPTNGYTRY DPKFQDKATI TADTSSNTAY LQVSRLTSED TAVYYCSRWG | 100 |
| GDGFYAMDYW GQGASVTVSS ASTKGPSVFP LAPSSKSTSG GTAALGCLVK | 150 |
| DYFPEPVTVS WNSGALTSGV HTFPAVLQSS GLYSLSSVVT VPSSSLGTQT | 200 |
| YICNVNHKPS NTKVDKRVEP KSCDKTHTCP PCPAPELVGG PSVFLLPPKP | 250 |
| KDTLMISRTP EVTCVVVDVS HEDPEVKFNW YVDGVEVHNA KTKPPEEQYN | 300 |
| STLRVVSVLT VLHQDWLNGK EYKCKVSNKA LPAPIEKTIS KAKGQPREPQ | 350 |
| VYTLPPSRDE LTKNQVSLTC LVKGFYPSDI AVEWESNGQP ENNYKTTPLV | 400 |
| LDSDGSFFLY SKLTVDKSRW QQGNVFSCSV MHEALHNHYT QKSLSLSPGK. | 450 |

### Margetuximab Light Chain

| (SEQ ID NO: 28) | |
|---|---|
| DIVMTQSHKF MSTSVGDRVS ITCKASQDVN TAVAWYQQKP GHSPKLLIYS | 50 |
| ASFRYTGVPD RFTGSRSGTD FTFTISSVQA EDLAVYYCQQ HYTTPPTFGG | 100 |
| GTKVEIKRTV AAPSVFIFPP SDEQLKSGTA SVVCLLNNFY PREAKVQWKV | 150 |
| DNALQSGNSQ ESVTEQDSKD STYSLSSTLT LSKADYEKHK VYACEVTHQG | 200 |
| LSSPVTKSFN RGEC. | 214 |

### Antibodies That Inhibit CD73

Exemplary anti-CD73 antibodies that can be used in accordance with the combination treatment methods described herein include an antibody or antibody fragment that binds a human CD73 polypeptide comprises VH and VL frameworks (e.g., FR1, FR2, FR3 and FR4) of human origin. In one aspect, the antibody or antibody fragment comprises: a HCDR1 (heavy chain CDR1) comprising an amino acid sequence SYNMY as set forth in SEQ ID NO: 2; a HCDR2 (heavy chain CDR2) comprising an amino acid sequence YIDPYNGGSSYNQKFKG as set forth in SEQ ID NO: 12, optionally further wherein the glutamine residue (Q) at position 13 of SEQ ID NO: 12 may be substituted by a leucine residue (L), wherein the lysine residue

(K) at position 14 of SEQ ID NO: 12 may optionally be substituted by a threonine residue (T); a HCDR3 (heavy chain CDR3) comprising an amino acid sequence GYNNYKAWFAY as set forth in SEQ ID NO: 13, optionally further wherein the asparagine residue (N) at position 3 of SEQ ID NO: 13 may be substituted by a glycine residue (G); a LCDR1 (light chain CDR1) comprising an amino acid sequence KASQSVTNDVA as set forth in SEQ ID NO: 14, optionally further wherein the threonine residue (T) at position 7 of SEQ ID NO: 14 may be substituted by a serine residue (S); a LCDR2 (light chain CDR2) comprising an amino acid sequence YASNRYT as set forth in SEQ ID NO: 15, optionally wherein the asparagine residue (N) at position 4 of SEQ ID NO: 15 may be substituted by an threonine residue (T); and a LCDR3 (light chain CDR3) comprising an amino acid sequence QQDYSSLT as set forth in SEQ ID NO: 7.

In one embodiment, a HCDR2 comprises an amino acid sequence of Formula I:

Y-I-D-P-Y-N-G-G-S-S-Y-N-Xaa1-Xaa2-F-K-G

(SEQ ID NO: 3), or a subsequence thereof, wherein Xaa1 is Q (Gln) or L (Leu) and wherein Xaa2 is K (Lys) or T (Thr).

In one embodiment, a HCDR3 comprises an amino acid sequence of Formula II:

G-Y-Xaa1-N-Y-K-A-W-F-A-Y

(SEQ ID NO: 4), or a subsequence thereof, wherein Xaa1 is N (Asp) or G (Gly).

In one embodiment, a LCDR1 comprises an amino acid sequence of Formula III:

K-A-S-Q-S-V-Xaa1-N-D-V-A

(SEQ ID NO: 5), or a subsequence thereof, wherein Xaa1 is T (Thr) or S (Ser).

In one embodiment, a LCDR2 comprises an amino acid sequence of Formula IV:

Y-A-S-Xaa1-R-Y-T

(SEQ ID NO: 6), or a subsequence thereof, wherein Xaa1 is T (Thr) or N (Asn).

In one aspect, an antibody that binds a human CD73 polypeptide for used in the treatment method herein comprises: a HCDR1 comprising an amino acid sequence SYNMY as set forth in SEQ ID NO: 2; a HCDR2 comprising an amino acid sequence YIDPYNGGSSYNLTFKG as set forth in SEQ ID NO: 8; a HCDR3 comprising an amino acid sequence GYGNYKAWFAY as set forth in SEQ ID NO: 9; a LCDR1 comprising an amino acid sequence KASQSVSNDVA as set forth in SEQ ID NO: 10; a LCDR2 comprising an amino acid sequence YASTRYT as set forth in SEQ ID NO: 11; and a LCDR3 comprising an amino acid sequence QQDYSSLT as set forth in SEQ ID NO: 7.

In one aspect, an antibody that binds a human CD73 polypeptide for used in the treatment method herein comprises: a HCDR1 comprising an amino acid sequence SYNMY as set forth in SEQ ID NO: 2; a HCDR2 comprising an amino acid sequence YIDPYNGGSSYNQKFKG as set forth in SEQ ID NO: 12; a HCDR3 comprising an amino acid sequence GYNNYKAWFAY as set forth in SEQ ID NO: 13; a LCDR1 comprising an amino acid sequence KASQSVTNDVA as set forth in SEQ ID NO: 14; a LCDR2 comprising an amino acid sequence YASNRYT as set forth in SEQ ID NO: 15; and a LCDR3 comprising an amino acid sequence QQDYSSLT as set forth in SEQ ID NO: 7.

In one embodiment, the antibody comprises a heavy chain framework from the human subgroup IGHV1-3 (optionally together with IGHJ4), optionally the IGHV1-3 is IGHV1-3*01. In one embodiment, the humanized antibody comprises a light chain framework from the human subgroup IGKV1-33 (optionally together with IGKJ2), optionally from IGKV1-33*01.

The antibody may further comprise one, two, three, four, five or more amino acid substitutions across the human heavy and/or light chain frameworks, to, e.g., enhance affinity, stability, or other properties of the antibody.

Examples of VH and VL amino acid sequences of anti-CD73 antibodies are shown in Example 2 (showing the H4+ chain, and Table 3 for 2H4+2Lx antibodies), and Table 1 for L1-L4 chains.

In one aspect, the anti-CD73 antibody or antibody fragment used in the treatment method herein comprises:
(a) a CDR-H1 comprising the amino acid sequence of SEQ ID NO: 2;
(b) a CDR-H2 comprising the amino acid sequence of SEQ ID NOS: 3, 8 or 12;
(c) a CDR-H3 comprising the amino acid sequence of SEQ ID NOS: 4, 9 or 13;
(d) a CDR-L1 comprising the amino acid sequence of SEQ ID NOS: 5, 10 or 14;
(e) a CDR-L2 comprising the amino acid sequence of SEQ ID NOS: 6, 11 or 15;
(f) a CDR-L3 comprising the amino acid sequence of SEQ ID NO: 7; and
(g) human heavy and light chain framework sequences.

In one embodiment, the antibody comprises a heavy chain framework from the human subgroup IGHV1-3 together with IGHJ4, optionally the antibodies comprises IGHV1-3*01, together with IGHJ4. In one embodiment, the humanized antibody comprises a light chain framework from the human subgroup IGKV1-33, optionally IGKV1-33*01, together with IGKJ2.

Optionally a human framework comprises one or more mutations, e.g., back mutations to introduce a residue present at the particular position in a non-human mammal (e.g., a mouse).

In one aspect of any embodiment herein, the amino acid at Kabat heavy chain position 2 is an isoleucine.

In one aspect of any embodiment herein, the amino acid at Kabat heavy chain position 30 is an alanine.

In one aspect of any embodiment herein, the amino acid at Kabat heavy chain position 48 is an isoleucine.

In one aspect of any embodiment herein, the amino acid at Kabat heavy chain position 69 is a leucine.

In one aspect of any embodiment herein, the amino acid at Kabat heavy chain position 73 is a lysine.

In one embodiment, a VH comprises an isoleucine residue at Kabat position 2, an alanine at position 30, an isoleucine at position 48, a leucine at position 69 and a lysine at position 73.

In one aspect of any embodiment herein, the amino acid at Kabat light chain position 67 is a tyrosine.

In one aspect of any embodiment herein, the amino acid at Kabat light chain position 60 is serine or aspartic acid.

In one aspect of any embodiment herein, the amino acid at Kabat light chain position 2 is valine or isoleucine.

In one aspect of any embodiment herein, the amino acid at Kabat light chain position 87 is tyrosine or phenylalanine.

In one embodiment, a VL comprises a tyrosine residue at Kabat position 67, a serine at position 60, an isoleucine at position 2, and a tyrosine at position 87.

In one aspect of any embodiment herein, the amino acid at Kabat heavy chain position 28 is threonine (T).

In one aspect of any embodiment herein, the amino acid at Kabat heavy chain position 66 is arginine (R).

In one aspect of any embodiment herein, the amino acid at Kabat heavy chain position 67 is valine (V).

In one aspect of any embodiment herein, the amino acid at Kabat heavy chain position 71 is arginine (R).

Positions in the VH and VL domains herein are described using the Kabat numbering system (Kabat et al. (1991) Sequences of Protein of Immunological Interest, 5th ed., United States Public Health Service, National Institute of Health, Bethesda, MD).

In one aspect, the anti-CD73 antibody comprises a VH domain having at least about 80% sequence identity (e.g., at least about 85%, 90%, 95%, 97%, 98% or 99% identity, or 100% identity) to the VH domain of SEQ ID NOS: 37 or 42.

In one aspect, the anti-CD73 antibody or antibody fragment comprises a VL domain having at least about 80% sequence identity (e.g., at least about 85%, 90%, 95%, 97%, 98% or 99% identity, or 100% identity) to the VL domain of any of SEQ ID NOS: 33-36 or 43-46.

In one aspect, the anti-CD73 antibody or antibody fragment comprises a VH domain having at least about 80% sequence identity (e.g., at least about 85%, 90%, 95%, 97%, 98% or 99% identity, or 100% identity) to the VH domain of SEQ ID NO: 42, and a VL domain having at least about 80% sequence identity (e.g., at least about 85%, 90%, 95%, 97%, 98% or 99% identity, or 100% identity) to the VL domain of any of SEQ ID NOS: 43-46.

In one aspect, the anti-CD73 antibody or antibody fragment comprises a heavy chain having at least about 80% sequence identity (e.g., at least about 85%, 90%, 95%, 97%, 98% or 99% identity, or 100% identity) to the heavy chain of SEQ ID NO: 38 of the H4+L1 antibody. In one embodiment, the antibody or antibody fragment comprises a light chain having at least about 80% sequence identity (e.g., at least about 85%, 90%, 95%, 97%, 98% or 99% identity, or 100% identity) to the light chain of SEQ ID NO: 39 of the H4+L1 antibody.

In one aspect, the anti-CD73 antibody or antibody fragment comprises a heavy chain having at least about 80% sequence identity (e.g., at least about 85%, 90%, 95%, 97%, 98% or 99% identity, or 100% identity) to the heavy chain of SEQ ID NO: 47 of the 2H4+2L1 antibody. In one embodiment, the antibody or antibody fragment comprises a light chain having at least about 80% sequence identity (e.g., at least about 85%, 90%, 95%, 97%, 98% or 99% identity, or 100% identity) to the light chain of SEQ ID NO: 48 of the 2H4+2L1 antibody.

In one aspect, the anti-CD73 antibody or antibody fragment comprises a heavy chain variable region comprising the amino acid sequence of SEQ ID NO: 42 and a light chain variable region comprising the amino acid sequence of SEQ ID NO: 43.

In one aspect, the anti-CD73 antibody or antibody fragment comprises a heavy chain comprising the amino acid sequence of SEQ ID NO: 47 and a light chain comprising the amino acid sequence of SEQ ID NO: 48.

In other aspects, an anti-CD73 antibody for use in accordance with the treatment methods of the invention can have heavy and light chain CDR1, 2 and 3 (e.g. as determined according to Kabat numbering), or heavy and light chain variable regions derived from another suitable anti-CD73 antibody.

Each subunit of the CD73 dimer consists of two structural domains: the N-terminal domain (residues 27-317, numbering as referenced in Knapp et al. (2012)) and the C-terminal domain (residues 337-549), with the larger N-terminal domain of CD73 containing the metal ion binding site and having a four-layered a/b-b-b-a structure, including two sandwiched mixed b sheets. The C-terminal domain contains the substrate binding site and dimerization interface, and has a four-layered structure of the composition a/b-b-a-b. The two domains are linked by a single a helix (residues 318-336) comprising a small hinge region, which enables the enzyme to undergo large domain movements and thereby switch between the open and closed conformations. The active site, observed in the closed conformation, is located at the interface between the N- and C-terminal domains and is formed from residues of both domains. See, e.g., Knapp et al. (2012) Structure 20, 2161-2173, the disclosure of which is incorporated herein by reference.

A class of suitable anti-CD73 antibodies of particular interest is represented by antibodies such as 11E1, 6E1, 3C12 and 8C7 (see WO2016/055609, Innate Pharma, the disclosure of which is incorporated herein by reference) that lack the “hook effect” observed with previous anti-CD73 antibodies. Other exemplary anti-CD73 antibodies reported to lack hook effect include antibodies 350, 356, 358, 373 (e.g. 373.A), 374, 376, 377, 379 or Hu101-28 (see WO2018/237157 and WO2018137598, the disclosures of which are incorporated herein by reference). The hook effect refers to tendency of anti-CD73 antibodies to present as false positives for inhibition of CD73 as a soluble dimeric protein. Such anti-CD73 antibodies, which are believed to cause cross-linking of CD73 dimers rather than true inhibition of enzymatic activity, lose the ability to inhibit CD73 when tested at high excess to CD73 dimers, as discussed in WO2016/055609. The antibodies such as 11E1, 6E1, 3C12 and 8C7 inhibit the enzymatic activity of soluble CD73 (by binding to bivalently to one CD73 dimer) and are able to act as particularly potent non-competitive inhibitors of cell membrane-bound CD73 (without reliance on internalization) have been (see PCT publication no. WO2016/055609). In view of an ability to substantially fully neutralize CD73 (e.g. in the tumor stroma), these antibodies can be advantageously used, in particular, in treatment regimens in combination with chemotherapy agents associated with induction of expression of CD73 in the tumor stroma and/or in combination with anti-HER2 antibodies.

Antibodies preferably bind an epitope present on CD73 expressed at the surface of cells, including tumor cells, and inhibit the enzymatic (ecto-5′ nucleotidase) activity of the CD73 enzyme (e.g. membrane-bound CD73 protein expressed at the surface of cells). In one embodiment, these antibodies can be used as pure CD73 blocking antibodies, e.g., they inhibit the enzymatic activity of membrane-bound CD73 protein expressed at the surface of cells without substantially binding Fcγ receptors and/or without substantially directing ADCC toward a CD73-expressing cell. Optionally, the antibodies retain an Fc domain and retain binding to human FcRn.

In one aspect, anti-CD73 antibodies may bind an epitope on CD73 that is present on CD73 not only when not bound to substrate but also when bound to a substrate (e.g. a natural substrate such as AMP or an inhibitor or other compound that binds the active site such as an AMP analogue adenosine 5′-(α,β-methylene)diphosphate (APCP)). In one aspect, an anti-CD73 antibody does not compete with a substrate of CD73 for binding to a CD73 polypeptide. Examples of substrates of CD73 include, e.g. a natural substrate such as AMP or an inhibitor or other compound that binds the active site such as an AMP analogue adenosine 5′-(α,β-methylene)diphosphate (APCP).

In one aspect, an anti-CD73 antibody acts as an allosteric inhibitors and binds to CD73 in an intra-dimer binding mode in a 1:1 stoichiometry between an intact full-length antibody and a CD73 dimer. In one aspect, the anti-CD73 antibody is capable of binding and constraining the CD73 polypeptide in an intermediate state (between the open (inactive) and closed (active, substrate-bound) states) in which AMP cannot be hydrolysed.

In one embodiment, the antibodies inhibit the enzymatic activity of CD73, when CD73 is present as a soluble recombinant CD73 protein, e.g. the antibodies are capable of inhibiting the enzymatic activity of soluble human dimeric CD73 polypeptide when the antibodies are in a setting/configuration where they not capable of forming oligomers, e.g. when they are provided at a substantial molar excess (e.g. at least 10-fold, 20-fold, 100-fold, etc.) to the CD73 polypeptide dimers. Because residual CD73 enzymatic activity can result in sufficient adenosine generation to mediate immunosuppressive effects, high levels of antibody-mediated enzyme blockade are advantageous in order to mediate a therapeutic effect.

Examples of such antibodies are described herein as well as in PCT publication no. WO2016/055609 (e.g. antibodies 11E1, 6E1, 3C12 and 8C7). Antibodies 11E1, 6E1, 3C12 and 8C7 lose binding to CD73 mutants having a substitution at residue K136 (with reference to the CD73 polypeptide of SEQ ID NO: 1). Antibodies 11E1, 6E1, 3C12 and 8C7 also lose binding to mutants having substitutions at residues A99, E129, K133, E134 and A135 (with reference to the CD73 polypeptide of SEQ ID NO: 1), as well as to mutants having a substitution at residues K97, E125, Q153 and K330 (with reference to the CD73 polypeptide of SEQ ID NO: 1). Other example of antibodies can bind in the segment of residues 131-162 of SEQ ID NO: 1, and in particular amino acid residues L131, K136, S155, L157, K162, K330 (with reference to the CD73 polypeptide of SEQ ID NO: 1).

Antibodies 11E1, 6E1, 3C12 and 8C7 are examples of antibodies that bind the CD73 dimer in an intra-dimer mode, constraining the CD73 enzyme in an inactive state in which AMP cannot not be hydrolyzed, in contrast to other antibodies that interact in an inter-dimer mode. Assays using soluble CD73 that can be used to identify such CD73 function blocking antibodies, are provided in PCT publication nos. WO2016/055609 and WO2016/131950. In one aspect, an anti-CD73 antibody is any antibody available or otherwise known at the filing date of the application disclosing the present invention, or an antibody fragment thereof (e.g. a fragment comprising the heavy and light chain CDRs) that retains the ability to bind CD73 and to inhibit the enzymatic activity of CD73.

Accordingly, an antibody may be an allosteric inhibitor of the CD73 polypeptide, e.g. the antibody binds human CD73 polypeptide expressed at the surface of a cell, including but limited to tumor cells, and inhibits the enzymatic (ecto-5′ nucleotidase) activity CD73 polypeptide, without interfering with the ability of a substrate of the CD73 polypeptide to bind the CD73 polypeptide.

Exemplary antibodies are described herein that bind to an epitope on CD73 that is present on the same face when CD73 is present as a CD73 dimer, e.g., potentially permitting an antibody to bind bivalently to one CD73 dimer, the antibody binds to CD73 in an intra-dimer binding mode in a 1:1 stoichiometry between an intact full-length antibody and a CD73 dimer. In view of binding to ligand-bound CD73, the antibodies described herein may be useful for binding to CD73 when bound to AMP, e.g., in the tumor environment where upstream ADP and/or AMP are present at significant levels prior to treatment). The tumor microenvironment can be characterized by any appropriate parameter, for example high levels of ADP (e.g. generated by dying cells), AMP, adenosine, by presence or levels of CD73 expression or CD73-expressing cells, by presence or levels of adenosine receptor expression or adenosine-receptor expressing cells. Thus, CD73 molecules in the tumor environment may be in the substrate-bound conformation and the ability to bind and inhibit substrate-bound cellular CD73 (e.g. cells expressing CD73 pre-incubated with substrate such as AMP) in addition to non- substrate bound CD73 may provide greater ability to inhibit CD73 in vivo. Optionally, levels of ADP or AMP (and/or ATP or adenosine) can be assessed in the tumor environment prior to treatment. The antibodies may have a particular advantage for treatment in an individual having significant levels (e.g. high levels, compared to a reference) ADP, AMP, ATP or adenosine in the tumor sample.

Exemplary antibodies may bind a human CD73 polypeptide expressed at the surface of cells and that inhibits the enzymatic (ecto-5′ nucleotidase) activity of the CD73 polypeptide, wherein the antibody is capable of binding bivalently to a single CD73 polypeptide dimer (a soluble CD73 polypeptide dimer or a CD73 polypeptide dimer expressed by a cell). Optionally, the antibody binds with a first antigen binding domain to a first CD73 polypeptide within the dimer and with a second antigen binding domain to a second CD73 polypeptide.

An exemplary antibody may bind a human CD73 polypeptide expressed at the surface of cells and inhibits the enzymatic (ecto-5′ nucleotidase) activity of the CD73 polypeptide, wherein the antibody is capable of binding the CD73 polypeptide in the substrate-bound conformation.

An anti-CD73 antibody can be assessed and selected for its ability to inhibit the enzymatic activity of CD73, notably to block the 5′-nucleotidase activity of CD73 and to reduce the production of adenosine by a CD73-expressing cell, and in turn restore the activity of and/or relieve the adenosine-mediated inhibition of lymphocytes.

The ability of an antibody to inhibit the enzymatic activity of CD73 can be tested in a cell-free assay using recombinant soluble human CD73 (as dimers) and AMP, where conversion of AMP to adenosine (and/or inhibition thereof) is detected directly (e.g. by measurement of substrates and products, i.e. AMP, adenosine and/or phosphate), or indirectly. In one example, AMP and/or adenosine are detected via HPLC before and after incubation of the test compound with recombinant CD73. Recombinant CD73 is described, e.g., in WO2016/055609 and WO2016/131950.

The inhibitory activity of an antibody can also be assessed in any of a number of other ways. For example, in an indirect assay, a luciferase-based reagent is used (e.g. CellTiter-Glo® system available from Promega), to detect the disappearance of AMP. The luciferase reaction in the assay is inhibited by AMP. Adding the CD73 enzyme to the reaction degrades the AMP, and relieves the inhibition, producing a detectable signal.

The assays using soluble CD73 can advantageously involve testing at conditions where the antibodies are provided at a substantial molar excess (e.g. 10-fold, 20-fold, 50-fold, 100-fold, etc.) to the CD73 polypeptide dimers. When provided in molar excess to the enzyme, the anti-CD73 antibodies will no longer be capable of forming multimeric complexes of antibodies and CD73 dimers; antibodies that retain inhibition of the enzymatic activity of CD73 can then be selected.

The ability of an antibody to inhibit the 5′-ectonucleotidase enzymatic activity of CD73 can alternatively or in addition also be tested in a cellular assay (using cells that express CD73). Advantageously, antibodies can be tested or screened first in the cell-free assay to identify antibodies that block the activity of the enzyme to reduce likelihood of selecting antibodies that inhibit CD73 by causing internalization of CD73, and then tested as purified antibody in cellular assays. Cellular assays can be carried out as shown in WO2016/055609. For example, a CD73-expressing cell line (e.g. MDA-MB-231 cell line) are plated in flat-bottom 96 well plates in presence of anti-CD73 antibodies and incubated. AMP is added to the cells and incubated at 4° C. (to avoid CD73 down-modulation). Plates are then centrifuged and supernatant is transferred to flat bottom 96 well culture plate. Free phosphate produced by the hydrolysis of AMP into adenosine is then quantified. A decrease in hydrolysis of AMP into adenosine in the presence of antibody indicate the antibody inhibits cellular CD73.

In one embodiment, an antibody preparation causes at least a 50% decrease in the enzymatic activity of a CD73 polypeptide, preferably at least a 60%, 70% or 80% decrease in the enzymatic activity of a CD73 polypeptide (e.g. a soluble homodimeric CD73 polypeptide; CD73 expressed by cells).

The activity of an antibody can also be measured in an indirect assay for its ability to modulate the activity of lymphocytes, for example to relieve the adenosine-mediated inhibition of lymphocyte activity, or to cause the activation of lymphocyte activity. This can be addressed, for example, using a cytokine-release assay. In another example, an antibody can be evaluated in an indirect assay for its ability to modulate the proliferation of lymphocytes.

The antibody can be tested for its ability to internalize or to induce down-modulation of CD73, e.g. whether by internalization or induction of CD73 shedding from the cell surface. Whether an anti-CD73 antibody internalizes upon binding CD73 on a mammalian cell, or whether a CD73 polypeptide undergoes intracellular internalization (e.g. upon being bound by an antibody) can be determined by various assays including those described in WO2016/055609, for example, the disclosure of which is incorporated herein by reference.

In one example, antibodies can be selected for the ability to inhibit the enzymatic activity of soluble human dimeric CD73 polypeptide when the antibodies are in a setting/configuration where they not capable of forming oligomers, e.g. when they are provided at a substantial molar excess (e.g. at least 10-fold, 20-fold, 100-fold, etc.) to the CD73 polypeptide dimers. Antibodies that function by causing oligomerization fail to inhibit CD73 when the antibodies provided at a substantial molar excess to the CD73 polypeptide dimers. The antibodies furthermore bind an epitope on CD73 that is maintained when CD73 is expressed at the cell surface. Through use of this assay, antibodies can also be identified that bind bivalently to a single CD73 dimer; such antibodies may have improved CD73-binding and CD73 blocking activity in vitro and vivo in CD73-expressing cells. The antibodies identified by these methods were then tested in cellular enzymatic activity assays using purified antibody, and found to neutralize the enzymatic activity of cellular CD73. Antibodies that inhibit CD73 by inducing primarily by inducing internalization or that lose significant binding to cellular CD73 were less potent and were not able to neutralize enzymatic activity, providing at best only partial inhibition of the enzymatic activity of CD73 in cells.

The epitope on CD73 bound by these antibodies is present on CD73 polypeptides as expressed by a range of cells, e.g. cancer cells, CD4 T cells, CD8 T cells, B cells, transfected cells, and binds with high affinity as determined by flow cytometry. For example, an antibody can be characterized by an EC₅₀, as determined by flow cytometry, that is comparable to, or of no more than 2-log, optionally 1-log, greater than that of an anti-CD73 antibody described herein (e.g., antibody 6E1), or of no more than 5 μg/ml, optionally no more than 2 μg/ml, no more than 1 μg/ml, no more than 0.5 μg/ml, no more than 0.1 μg/ml or no more than 0.05 μg/ml, for binding to cells that express at their surface a CD73 polypeptide. In one embodiment the cells are cells that are made to express CD73 at their surface. In one embodiment the cells are cells that endogenously express CD73 at their surface, e.g. cancer cells.

In one embodiment, the CD73 neutralizing antibodies can be characterized by being capable of causing a decrease in cells' 5′-ectonucleotidase activity of CD73 by at least 60%, 75% or 80%. In one embodiment, the CD73-neutralizing antibodies can be characterized by an EC₅₀ for inhibition of 5′-ectonucleotidase activity of CD73 expressed by a cell that is comparable to, or of no more than that of an antibody described herein, of no more than 2-log, optionally 1-log, greater than that of an anti-CD73 antibody described herein (e.g., antibody 6E1), or no more than 1 μg/ml, optionally no more than 0.5 μg/ml, optionally no more than 0.2 μg/ml.

Optionally, inhibition of 5′-ectonucleotidase activity of CD73 expressed by a cell is determined by assessing neutralization of 5′ ectonucleotidase activity in MDA-MB-231 cells by quantifying hydrolysis of AMP to adenosine (see, e.g., Example 5 of WO2016/055609).

In one embodiment, an antibody specifically binds human CD73 at the surface of a cell and is capable of neutralizing the 5′-ectonucleotidase activity of cellular CD73 (CD73 expressed by cells). In one embodiment, an antibody specifically binds and neutralizes the 5′-ectonucleotidase activity of a human CD73 at the surface of a cell, and is not internalized into CD73-expressing cells upon binding to CD73, e.g., the antibody does not require for its CD73-neutralizing activity the multimerization and subsequence internalization of CD73. In one embodiment, the antibody is a non-depleting antibody, e.g., an Fc silent antibody. The antibody is capable of neutralizing the 5′-ectonucleotidase activity of a dimeric human CD73 polypeptide in solution, moreover without reliance on induction of oligomers of CD73 polypeptides anti-CD73 antibodies.

In one aspect, an antibody specifically binds human CD73 at the surface of a cell pre-incubated with AMP, and is capable of neutralizing the 5′-ectonucleotidase activity thereof. Optionally, neutralizing the 5′-ectonucleotidase activity is determined by assessing neutralization of 5′ ectonucleotidase activity in MDA-MB-231 cells by quantifying hydrolysis of AMP to adenosine (see, e.g., Example 5 of WO2016/055609).

Optionally, an anti-CD73 antibody can bind to a common antigenic determinant present on both soluble CD73 and CD73 expressed at the cell surface.

Optionally, an anti-CD73 antibody binds a common antigenic determinant present on CD73 when CD73 active site is not occupied by/bound to a substrate, e.g. AMP, APCP) and when CD73 active site is occupied by/bound to a substrate, e.g. AMP, APCP).

In one aspect, an anti-CD73 antibody binds an antigenic determinant within each CD73 polypeptide chain within a CD73 dimer, e.g., wherein the antigenic determinants are present on a common face of the CD73 dimer.

In one aspect, an anti-CD73 antibody has reduced binding to a CD73 polypeptide having an amino acid substitution at a residue in segment 158-171 and/or at a residue in segment 206-211, e.g. a CD73 polypeptide having an amino acid substitution at any one or more residues V170, K206 and N211 (reference to SEQ ID NO: 1).

In one aspect, an anti-CD73 antibody has reduced binding to a CD73 polypeptide having an amino acid substitution at a residue in segment 65-83 and/or at a residue in segment 157-172 (reference to SEQ ID NO: 1).

In one aspect, an anti-CD73 antibody binds an epitope on CD73 comprising residue K136 (with reference to SEQ ID NO: 1).

In one aspect, an anti-CD73 antibody binds an epitope on CD73 comprising one, two, three or four of the residues selected from the group consisting of K97, E125, Q153 and K330 (with reference to SEQ ID NO: 1).

In one aspect, an anti-CD73 antibody binds that bind an epitope on CD73 comprising one, two, three, four or five of the residues selected from the group consisting of A99, E129, K133, E134, and A135 (with reference to SEQ ID NO: 1).

In one aspect, an anti-CD73 antibody binds that bind an epitope on CD73 comprising one, two, three, four or five of the residues selected from the group consisting of Y345, D399, E400, R401 and R480 (with reference to SEQ ID NO: 1).

In one aspect, an anti-CD73 antibody binds at least partly within a domain or segment of amino acid residues on a human CD73 protein (e.g. a CD73 homodimer protein) comprising the amino acid residues K97, A99, E125, E129, K133, E134, A135, K136, Q153 and K330 (with reference to SEQ ID NO: 1). In one aspect, an anti-CD73 antibody binds an epitope on CD73 comprising at least one, two, three, four or five, or more, of the residues selected from the group consisting of K97, A99, E125, E129, K133, E134, A135, K136, Q153 and K330 (with reference to SEQ ID NO: 1).

In one aspect, an anti-CD73 antibody has reduced binding to a CD73 polypeptide having a mutation at a residue K136 (with reference to SEQ ID NO: 1); optionally, the mutant CD73 polypeptide has the mutation: K136A.

In one aspect, an anti-CD73 antibody has reduced binding to a CD73 polypeptide having a mutation at a residue selected from the group consisting of: K97, E125, Q153 and K330 (with reference to SEQ ID NO: 1); optionally, the mutant CD73 polypeptide has the mutations: K97A, E125A, Q153A and/or K330A (e.g., K97A, E125A and K330A; K97A, E125A and/or Q153A).

In one aspect, an anti-CD73 antibody has reduced binding to a CD73 polypeptide having a mutation at a residue selected from the group consisting of: A99, E129, K133, E134, and A135 (with reference to SEQ ID NO: 1); optionally, the mutant CD73 polypeptide has the mutations: A99S, E129A, K133A, E134N, and A135S.

In one aspect, an anti-CD73 antibody binds that bind an epitope on CD73 comprising one, two, three, four, five or six of the residues selected from the group consisting of L131, K136, S155, L157, K162, and K330 (with reference to SEQ ID NO: 1).

In one aspect, an anti-CD73 antibody has reduced binding to a CD73 polypeptide having a mutation at one or more (or all of) residues selected from the group consisting of: L131, K136, S155, L157 and K162 (with reference to SEQ ID NO: 1).

In one aspect, an anti-CD73 antibody has reduced binding to a CD73 polypeptide having a mutation at one or more (or all of) residues selected from the group consisting of: L131, K136, S155, L157, K162 and K330 (with reference to SEQ ID NO: 1).

In one aspect, an anti-CD73 antibody has reduced binding to a CD73 polypeptide having a mutation at one or more (or all of) residues selected from the group consisting of: Y345, D399, E400, R401 and R480 (with reference to SEQ ID NO: 1).

In one aspect an anti-CD73 antibody competes for binding to an epitope on CD73 bound by antibody 11E1, 8C7, 3C12, 6E1, 373.A and/or Hu101-28, (e.g., that competes for binding to an epitope on a CD73 polypeptide with an antibody having the heavy and light chain CDRs or variable regions of said antibody).

In one aspect of any of the embodiments herein, an antigen-binding compound binds the same epitope and/or competes for binding to an epitope on a CD73 polypeptide with monoclonal antibodies 11E1, 8C7, 3C12 and/or 6E1 (e.g., that competes for binding to a CD73 polypeptide with an antibody having the heavy and light chain CDRs or variable regions of any of 11E1, 8C7, 3C12 or 6E1). In one embodiment, an antigen-binding compound binds the same epitope and/or competes for binding to an epitope on a CD73 polypeptide with an antibody selected from the group consisting of:
(a) an antibody having respectively a VH and VL region of SEQ ID NOS: 49 and 50 (6E1);
(b) an antibody having respectively a VH and VL region of SEQ ID NOS: 51 and 52 (11E1);
(c) an antibody having respectively a VH and VL region of SEQ ID NOS: 53 and 54 (8C7); and
(d) an antibody having respectively a VH and VL region of SEQ ID NOS: 55 and 56 (3C12).

In one embodiment, an anti-CD73 antibody binds an epitope comprising one, two or three amino acid residues selected from the group consisting of the amino acid residues on CD73 bound by 11E1, 6E1, 3C12 or 8C7.

In one aspect of any of the embodiments herein, the antibody may have a heavy and/or light chain having one, two or three CDRs of the respective heavy and/or light chain of an antibody selected from the group of antibodies consisting of 11E1, 6E1, 3C12, 8C7, 373 (e.g. 373.A), or Hu101-28.

The amino acid sequence of the heavy and light chain variable regions of antibodies 11E1, 6E1, 3C12, 8C7, 373.A and Hu101-28 are listed in Table A. In a specific embodiment, the disclosure provides an antibody that binds the same or essentially the same epitope or determinant as monoclonal antibodies 11E1, 6E1, 3C12, 8C7, 373.A or Hu101-28; optionally the antibody comprises the hypervariable region of antibody 11E1, 6E1, 3C12, 8C7, 373.A or Hu101-28. In any of the embodiments herein, antibody 11E1, 6E1, 3C12, 8C7, 373.A or Hu101-28 can be characterized by the amino acid sequences and/or nucleic acid sequences encoding it. According to one embodiment, an antibody may comprise the three CDRs (e.g., according to Kabat, Chothia or IGMT numbering) of the heavy chain variable region of 11E1, 6E1, 3C12, 8C7, 373.A or Hu101-28. Also provided is a monoclonal antibody that further comprises the variable light chain variable region of 11E1, 6E1, 3C12, 8C7, 373.A or Hu101-28 or one, two or three of the CDRs (e.g., according to Kabat, Chothia or IGMT numbering) of the light chain variable region of the respective 11E1, 6E1, 3C12, 8C7, 373.A or Hu101-28 antibody. Optionally any one or more of said light or heavy chain CDRs may contain one, two, three, four or five or more amino acid modifications (e.g. substitutions, insertions or deletions). Optionally, provided is an antibody where any of the light and/or heavy chain variable regions comprising part or all of an antigen binding region of antibody 11E1, 6E1, 3C12, 8C7, 373.A or Hu101-28 are fused to an immunoglobulin constant region of the human IgG type, optionally a human constant region, optionally a human IgG1, IgG2, IgG3 or IgG4 isotype. In one embodiment, a human constant region optionally further comprising an amino acid substitution to reduce effector function (binding to human Fcγ receptors). In one embodiment, a human constant region (optionally a hinge region) optionally further comprising an amino acid substitution to increase or induce intracellular internalization of CD73.

In another aspect of any of the embodiments herein, any of the CDRs 1, 2 and/or 3 of the heavy and light chains may be characterized by a sequence of at least 4, 5, 6, 7, 8, 9 or 10 contiguous amino acids thereof, and/or as having an amino acid sequence that shares at least 50%, 60%, 70%, 80%, 85%, 90% or 95% sequence identity with the particular CDR or set of CDRs (e.g. listed in the SEQ ID NO). In any of the antibodies, e.g., 11E1, 8C7, 3C12 or 6E1, the specified variable region and CDR sequences may comprise sequence modifications, e.g. a substitution (1, 2, 3, 4, 5, 6, 7, 8 or more sequence modifications). In one embodiment, a

CDRs 1, 2 and/or 3 of the heavy and light chains comprises one, two, three or more amino acid substitutions, where the residue substituted is a residue present in a sequence of human origin. In one embodiment the substitution is a conservative modification. A conservative sequence modification refers to an amino acid modification that does not significantly affect or alter the binding characteristics of the antibody containing the amino acid sequence. Such conservative modifications include amino acid substitutions, additions and deletions. Modifications can be introduced into an antibody by standard techniques known in the art, such as site-directed mutagenesis and PCR-mediated mutagenesis. Conservative amino acid substitutions are typically those in which an amino acid residue is replaced with an amino acid residue having a side chain with similar physicochemical properties. Specified variable region and CDR sequences may comprise one, two, three, four or more amino acid insertions, deletions or substitutions. Where substitutions are made, preferred substitutions will be conservative modifications. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g. glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g. threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for retained function (i.e., the properties set forth herein) using the assays described herein.

The HCDR1, 2, 3 and LCDR1, 2, 3 of an antibody having the VH and VL sequences indicated in Table A can optionally be specified as all (or each, independently) being those of the Kabat numbering system, those of the Chothia numbering system, those of the IMGT numbering system, or any other suitable numbering system. Underlined and bold sequences indicate Kabat CDRs.

TABLE A

| | SEQ ID NO: | Amino Acid Sequence of Anti-CD73 antibodies |
|---|---|---|
| 6E1 VH | 49 | EFQLQQSGPELVKPGASVKVSCKASGYAFTSYNMYWVKQSHGKRLEWIG |
| | | YIDPYNGGSSYNQKFKGKATLTVDKSSSTAYMHLNNLTSEDSAVYYCAR |
| | | GYNNYKAWFAYWGQGTLVTVSA |
| 6E1 VL | 50 | SIVMTQTPKFLLVSAGDRVTITCKASQSVINDVAWYQQKPGQSPKLLIY |
| | | YASNRYTGVPDRFTGSGYGTDFTFTISTMQAEDLAVYFCQQDYSSLTFG |
| | | AGTKLELK |
| 11E1 VH | 51 | EIQLQQSGPELVKPGASVKVSCKASGYAFTSYNMYWVKQSHGKSLEWIG |
| | | YIDPYNGGTSYNQKFKGKATLTVDKSSSTAYMHLNSLTSEDSAVYYCAR |
| | | GYGNYKAWFAYWGQGTLVTVSA |
| 11E1 VL | 52 | DAVMTQTPKFLLVSAGDRVTITCKASQSVTNDVAWYQQKPGQSPKLLIY |
| | | YASNRYTGVPDRFTGSGYGTDFTFTISTVQAEDLAVYFCQQDYSSLTFG |
| | | AGTKLELK |
| 8C7 VH | 53 | EVQLQQSGPELVKPGASVKVSCKASGYAFASYNMNWVKQSHGKSLDWIG |
| | | YIDPYNGGSSYNLTFKGKATLTVDKSSTTAYMHLNSLTSEDSAVYYCAR |
| | | GYGNYKAWFAYWGQGTLVTVSAASTKGP |
| 8C7 VL | 54 | SIVMTPTPKFLLVSAGDRVTITCKASQSVSNDVAWYQQKPGQSPKLLIY |
| | | YASTRYTGVPDRFTGSGYGTDFTFTISTVQAEDLAVYFCQQDYSSLTFG |
| | | AGTKLELKRTVAAP |
| 3C12 VH | 55 | QIQLQQSGPELVKPGASVKVSCKASGYAFASYNMNWVKQSHGKSLDWIG |
| | | YIDPYNGGSSYNLTFKGKATLTVDKSSTTAYMHLNSLTSEDSAVYYCAR |
| | | GYGNYKAWFAYWGQGTLVTVSAASTKGP |
| 3C12 VL | 56 | DVVMTQTPKFLLVSAGDRVTITCKASQSVSNDVAWYQQKPGQSPKLLIY |
| | | YASTRYTGVPDRFTGSGYGTDFTFTISTVQAEDLAVYFCQQDYSSLTFG |
| | | AGTKLELKRTVAAP |
| 373.A VH | 57 | EVQLLESGGGLVQPGGSLRLSCAASGFTFHRYAMSWVRQAPGKGLEWVS |
| | | AISGSGMNTYYADSVKGRFTISRDNSKNTLYLQMNSLRAEDTAVYYCAR |
| | | GGLYGSGSYLSDFDLWGRGTLVTVSS |
| 373.A VL | 58 | EIVLTQSPATLSVSPGERATLSCRASQSVGSNLAWYQQKPGQAPRLLIY |
| | | GASTRATGIPARFSGSGSGTEFTLTISSLQSEDFAVYYCQQHNAFPYTF |
| | | GGGTKVEIK |
| Hu101-28 | 59 | EVQLQESGPGLVKPSETLSLTCAVSGYSITSGYYWNWIRQPPGKKLEWM |
| VH | | GYINYGGSNGYNPSLKSRITISRDTSKNQFSLKLSSVTAADTAVYYCAR |
| | | DYDAYYEALDDWGQGTTVTVSS |
| Hu101-28 | 60 | EIVLSQSPATLSLSPGERATLSCRASSRVNYMHWYQQKPGQSPRPWISA |
| VL | | TSNLASGVPARFSGSGSGTSYTLTISSLEPEDFAVYYCQQWSSNPPTFG |
| | | GGTKVEIK |

In one embodiment, the anti-CD73 antibodies can be prepared such that they do not have substantial binding to human Fcγ receptors, e.g., any one or more of CD16A, CD16B, CD32A, CD32B and/or CD64). Such antibodies may comprise constant regions of various heavy chains that are known to lack or have low binding to Fcγ receptors. Alternatively, antibody fragments that do not comprise (or comprise portions of) constant regions, such as F(ab′)2 fragments, can be used to avoid Fc receptor binding. Fc receptor binding can be assessed according to methods known in the art, including for example testing binding of an antibody to Fc receptor protein in a BIACORE assay. Also, generally any antibody IgG isotype can be used in which the Fc portion is modified (e.g., by introducing 1, 2, 3, 4, 5 or more amino acid substitutions) to minimize or eliminate binding to Fc receptors (see, e.g., WO 03/101485, the disclosure of which is herein incorporated by reference). Assays such to assess Fc receptor binding are well known in the art, and are described in, e.g., WO 03/101485.

In one embodiment, the anti-CD73 antibody can comprise one or more specific mutations in the Fc region that result in “Fc silent” antibodies that have minimal interaction with effector cells. Silenced effector functions can be obtained by mutation in the Fc region of the antibodies and have been described in the art: N297A mutation, the LALA mutations, (Strohl, W., 2009, Curr. Opin. Biotechnol. Vol. 20(6):685-691); and D265A (Baudino et al., 2008, J. Immunol. 181: 6664-69) see also Heusser et al., WO2012/065950, the disclosures of which are incorporated herein by reference. In one embodiment, an antibody comprises one, two, three or more amino acid substitutions in the hinge region. In one embodiment, the antibody is an IgG1 or IgG2 and comprises one, two or three substitutions at residues 233-236, optionally 233-238 (EU numbering). In one embodiment, the antibody is an IgG4 and comprises one, two or three substitutions at residues 327, 330 and/or 331 (EU numbering). Examples of silent Fc IgG1 antibodies are the LALA mutant comprising L234A and L235A mutation in the IgG1 Fc amino acid sequence. Another example of an Fc silent mutation is a mutation at residue D265, or at D265 and P329 for example as used in an IgG1 antibody as the DAPA (D265A, P329A) mutation (U.S. Pat. No. 6,737,056). Another silent IgG1 antibody comprises a mutation at residue N297 (e.g., N297A, N297S mutation), which results in aglycosylated/non-glycosylated antibodies. Other silent mutations include: substitutions at residues L234 and G237 (L234A/G237A); substitutions at residues S228, L235 and R409 (S228P/L235E/R409K,T,M,L); substitutions at residues H268, V309, A330 and A331 (H268Q/V309L/A330S/A331S); substitutions at residues C220, C226, C229 and P238 (C220S/C226S/C229S/P238S); substitutions at residues C226, C229, E233, L234 and L235 (C226S/C229S/E233P/L234V/L235A; substitutions at residues K322, L235 and L235 (K322A/L234A/L235A); substitutions at residues L234, L235 and P331 (L234F/L235E/P331S); substitutions at residues 234, 235 and 297; substitutions at residues E318, K320 and K322 (L235E/E318A/K320A/K322A); substitutions at residues (V234A, G237A, P238S); substitutions at residues 243 and 264; substitutions at residues 297 and 299; substitutions such that residues 233, 234, 235, 237, and 238 defined by the EU numbering system, comprise a sequence selected from PAAAP, PAAAS and SAAAS (see WO2011/066501).

In one embodiment, the anti-CD73 antibody can comprise one or more specific mutations in the Fc region. For example, such an antibody can comprise an Fc domain of human IgG1 origin, comprises a mutation at Kabat residue(s) 234, 235, 237, 330 and/or 331. One example of such an Fc domain comprises substitutions at Kabat residues L234, L235 and P331 (e.g., L234A/L235E/P331S or (L234F/L235E/P331S). Another example of such an Fc domain comprises substitutions at Kabat residues L234, L235, G237 and P331 (e.g., L234A/L235E/G237A/P331S). Another example of such an Fc domain comprises substitutions at Kabat L234, residues L235, A330 G237, and P331 (e.g., L234A/L235E/G237A/A330S/P331S). In one embodiment, the antibody comprises an Fc domain, optionally of human IgG1 isotype, comprising: a L234X1 substitution, a L235X2 substitution, and a P331X3 substitution, wherein X1 is any amino acid residue other than leucine, X2 is any amino acid residue other than leucine, and X3 is any amino acid residue other than proline; optionally wherein X1 is an alanine or phenylalanine or a conservative substitution thereof; optionally wherein X2 is glutamic acid or a conservative substitution thereof; optionally wherein X3 is a serine or a conservative substitution thereof. In another embodiment, the antibody comprises an Fc domain, optionally of human IgG1 isotype, comprising: a L234X1 substitution, a L235X2 substitution, a G237X4 substitution and a P331X4 substitution, wherein X1 is any amino acid residue other than leucine, X2 is any amino acid residue other than leucine, X3 is any amino acid residue other than glycine, and X4 is any amino acid residue other than proline; optionally wherein X1 is an alanine or phenylalanine or a conservative substitution thereof; optionally wherein X2 is glutamic acid or a conservative substitution thereof; optionally, X3 is alanine or a conservative substitution thereof; optionally X4 is a serine or a conservative substitution thereof. In another embodiment, the antibody comprises an Fc domain, optionally of human IgG1 isotype, comprising: a L234X1 substitution, a L235X2 substitution, a G237X4 substitution, G330X4 substitution, and a P331X5 substitution, wherein X1 is any amino acid residue other than leucine, X2 is any amino acid residue other than leucine, X3 is any amino acid residue other than glycine, X4 is any amino acid residue other than alanine, and X5 is any amino acid residue other than proline; optionally wherein X1 is an alanine or phenylalanine or a conservative substitution thereof; optionally wherein X2 is glutamic acid or a conservative substitution thereof; optionally, X3 is alanine or a conservative substitution thereof; optionally, X4 is serine or a conservative substitution thereof; optionally X5 is a serine or a conservative substitution thereof.

In the shorthand notation used here, the format is: Wild type residue: Position in polypeptide: Mutant residues, wherein residue positions are indicated according to EU numbering according to Kabat.

In one embodiment, an anti-CD73 antibody comprises a heavy chain constant region comprising the amino acid sequence below, or an amino acid sequence at least 90%, 95% or 99% identical thereto but retaining the amino acid residues at Kabat positions 234, 235 and 331 (underlined):

(SEQ ID NO: 16)
A S T K G P S V F P L A P S S K S T S G G T A A L
G C L V K D Y F P E P V T V S W N S G A L T S G V
H T F P A V L Q S S G L Y S L S S V V T V P S S S
L G T Q T Y I C N V N H K P S N T K V D K R V E P
K S C D K T H T C P P C P A P E A E G G P S V F L
F P P K P K D T L M I S R T P E V T C V V V D V S
H E D P E V K F N W Y V D G V E V H N A K T K P R
E E Q Y N S T Y R V V S V L T V L H Q D W L N G K
E Y K C K V S N K A L P A S I E K T I S K A K G Q
P R E P Q V Y T L P P S R E E M T K N Q V S L T C
L V K G F Y P S D I A V E W E S N G Q P E N N Y K
T T P P V L D S D G S F F L Y S K L T V D K S R W
Q Q G N V F S C S V M H E A L H N H Y T Q K S L S
L S P G K.

In one embodiment, an anti-CD73 antibody comprises a heavy chain constant region comprising the amino acid sequence below, or an amino acid sequence at least 90%, 95% or 99% identical thereto but retaining the amino acid residues at Kabat positions 234, 235 and 331 (underlined):

(SEQ ID NO: 17)
A S T K G P S V F P L A P S S K S T S G G T A A L
G C L V K D Y F P E P V T V S W N S G A L T S G V
H T F P A V L Q S S G L Y S L S S V V T V P S S S
L G T Q T Y I C N V N H K P S N T K V D K R V E P
K S C D K T H T C P P C P A P E F E G G P S V F L
F P P K P K D T L M I S R T P E V T C V V V D V S
H E D P E V K F N W Y V D G V E V H N A K T K P R
E E Q Y N S T Y R V V S V L T V L H Q D W L N G K
E Y K C K V S N K A L P A S I E K T I S K A K G Q
P R E P Q V Y T L P P S R E E M T K N Q V S L T C
L V K G F Y P S D I A V E W E S N G Q P E N N Y K
T T P P V L D S D G S F F L Y S K L T V D K S R W
Q Q G N V F S C S V M H E A L H N H Y T Q K S L S
L S P G K.

In one embodiment, an anti-CD73 antibody comprises a heavy chain constant region comprising the amino acid sequence below, or an amino acid sequence at least 90%, 95% or 99% identical thereto but retaining the amino acid residues at Kabat positions 234, 235, 237, 330 and 331 (underlined):

(SEQ ID NO: 18)
A S T K G P S V F P L A P S S K S T S G G T A A L
G C L V K D Y F P E P V T V S W N S G A L T S G V
H T F P A V L Q S S G L Y S L S S V V T V P S S S
L G T Q T Y I C N V N H K P S N T K V D K R V E P
K S C D K T H T C P P C P A P E A E G A P S V F L
F P P K P K D T L M I S R T P E V T C V V V D V S
H E D P E V K F N W Y V D G V E V H N A K T K P R
E E Q Y N S T Y R V V S V L T V L H Q D W L N G K
E Y K C K V S N K A L P S S I E K T I S K A K G Q
P R E P Q V Y T L P P S R E E M T K N Q V S L T C
L V K G F Y P S D I A V E W E S N G Q P E N N Y K
T T P P V L D S D G S F F L Y S K L T V D K S R W
Q Q G N V F S C S V M H E A L H N H Y T Q K S L S
L S P G K.

In one embodiment, an antibody comprises a heavy chain constant region comprising the amino acid sequence below, or a sequence at least 90%, 95% or 99% identical thereto but retaining the amino acid residues at Kabat positions 234, 235, 237 and 331 (underlined):

(SEQ ID NO: 19)
A S T K G P S V F P L A P S S K S T S G G T A A L
G C L V K D Y F P E P V T V S W N S G A L T S G V
H T F P A V L Q S S G L Y S L S S V V T V P S S S
L G T Q T Y I C N V N H K P S N T K V D K R V E P
K S C D K T H T C P P C P A P E A E G A P S V F L
F P P K P K D T L M I S R T P E V T C V V V D V S
H E D P E V K F N W Y V D G V E V H N A K T K P R
E E Q Y N S T Y R V V S V L T V L H Q D L W N G K
E Y K C K V S N K A L P A S I E K T I S K A K G Q
P R E P Q V Y T L P P S R E E M T K N Q V S L T C
L V K G F Y P S D I A V E W E S N G Q P E N N Y K
T T P P V L D S D G S F F L Y S K L T V D K S R W
Q Q G N V F S C S V M H E A L H N H Y T Q K S L S
L S P G K.

Fc silent antibodies result in no or low ADCC activity, meaning that an Fc silent antibody exhibits an ADCC activity that is below 50% specific cell lysis. Preferably an antibody substantially lacks ADCC activity, e.g., the Fc silent antibody exhibits an ADCC activity (specific cell lysis) that is below 5% or below 1%. Fc silent antibodies can also result in lack of FcγR-mediated cross-linking of CD73 at the surface of a CD73-expressing cell.

In one embodiment, the anti-CD73 antibody has a substitution in a heavy chain constant region at any one, two, three, four, five or more of residues selected from the group consisting of: 220, 226, 229, 233, 234, 235, 236, 237, 238, 243, 264, 268, 297, 298, 299, 309, 310, 318, 320, 322, 327, 330, 331 and 409 (numbering of residues in the heavy chain constant region is according to EU numbering according to Kabat). In one embodiment, the antibody comprises a substitution at residues 234, 235 and 322. In one embodiment, the antibody comprises a substitution at residues 234, 235 and 331. In one embodiment, the antibody comprises a substitution at residues 234, 235, 237 and 331. In one embodiment, the antibody comprises a substitution at residues 234, 235, 237, 330 and 331. In one embodiment, the Fc domain is of human IgG1 subtype. Amino acid residues are indicated according to EU numbering according to Kabat.

Optionally, in any embodiment herein, an anti-CD73 antibody can be characterized as being a function-conservative variant of any of the antibodies, heavy and/or light chains, CDRs or variable regions thereof described herein. “Function-conservative variants” are those in which a given amino acid residue in a protein or antibody has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein (e.g. heavy or light chains, or CDRs or variable regions thereof) to which it is compared. In one embodiment, the antibody comprises a heavy chain variable region that is a function-conservative variant of the heavy chain variable region of antibody 11E1, 6E1, 3C12 or 8C7, and a light chain variable region that is a function-conservative variant of the light chain variable region of the respective 11E1, 6E1, 3C12 or 8C7 antibody. In one embodiment, the antibody comprises a heavy chain that is a function-conservative variant of the heavy chain variable region of antibody 11E1, 6E1, 3C12 or 8C7 fused to a human heavy chain constant region disclosed herein, optionally a human IgG4 constant region, optionally a modified IgG (e.g. IgG1) constant region, e.g. a constant region disclosed herein, and a light chain that is a function-conservative variant of the light chain variable region of the respective 11E1, 6E1, 3C12 or 8C7 antibody fused to a human Ckappa light chain constant region.

### Kits and Formulations

Any of the active agents, e.g., anti-CD73 antibodies, anti-HER2 antibodies or chemotherapy agents, can be specified as being embodied in a composition, such as pharmaceutically acceptable compositions and kits. A pharmaceutically acceptable composition will typically comprise one or more additional ingredients that can be active ingredients or inactive ingredients that promote formulation, delivery, stability, or other characteristics of the composition (e.g., various carriers).

In a further embodiment, provided is a pharmaceutical formulation comprising a therapeutically effective amount of an anti-CD73, for use in treating a subject who has a gastric cancer (optionally a gastric adenocarcinoma or gastroesophageal junction adenocarcinoma), wherein the subject has a cancer that is HER2-positive, and wherein said pharmaceutical formulation is administered in combination with (e.g. in a combination treatment with) an anti-HER2 antibody and optionally further a chemotherapy agent (e.g. paclitaxel). In a further embodiment, provided is a pharmaceutical formulation comprising a therapeutically effective amount of an anti-HER2 antibody, for use in treating a subject who has a gastric cancer (optionally a gastric adenocarcinoma or a gastroesophageal junction adenocarcinoma), wherein the subject has a HER2-positive cancer, and wherein said pharmaceutical formulation is administered in combination with (e.g. in a combination treatment with) an anti-CD73 antibody and optionally further a chemotherapy agent (e.g. paclitaxel).

The terms “pharmaceutical composition” or “therapeutic composition” as used herein refer to a compound or composition capable of inducing a desired therapeutic effect when properly administered to a subject. In some embodiments, the disclosure provides a pharmaceutical composition comprising a pharmaceutically acceptable carrier and a therapeutically effective amount of at least one inhibitor of the disclosure. The terms “pharmaceutically acceptable carrier” or “physiologically acceptable carrier” as used herein refer to one or more formulation materials suitable for accomplishing or enhancing the delivery of one or more agents of the disclosure.

In some embodiments, the agents disclosed herein may be formulated with a pharmaceutically acceptable carrier, excipient, or stabilizer, as pharmaceutical compositions. In certain embodiments, such pharmaceutical compositions are suitable for administration to a human or non-human animal via any one or more routes of administration using methods known in the art. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also contain compatible solid or liquid fillers, diluents or encapsulating substances which are suitable for administration into a human. Other contemplated carriers, excipients, and/or additives, which may be utilized in the formulations described herein include, for example, flavoring agents, antimicrobial agents, sweeteners, antioxidants, antistatic agents, lipids, protein excipients such as serum albumin, gelatin, casein, salt-forming counterions such as sodium, and the like. These and additional known pharmaceutical carriers, excipients, and/or additives suitable for use in the formulations described herein are known in the art, e.g., as listed in “Remington: The Science & Practice of Pharmacy,” 21st ed., Lippincott Williams & Wilkins, (2005), and in the “Physician's Desk Reference,” 60th ed., Medical Economics, Montvale, N.J. (2005). Pharmaceutically acceptable carriers can be selected that are suitable for the mode of administration, solubility, and/or stability desired or required.

In one embodiment, the formulations of the disclosure are pyrogen-free formulations that are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension, and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one-hour period for intravenous drug applications (The United States Pharmacopeial Convention, Pharmacopeial Forum 26(1): 223 (2000)). In certain embodiments, the endotoxin and pyrogen levels in the composition are less than 10 EU/mg, or less than 5 EU/mg, or less than 1 EU/mg, or less than 0.1 EU/mg, or less than 0.01 EU/mg, or less than 0.001 EU/mg.

When used for in vivo administration, the formulations of the disclosure should be sterile. The formulations of the disclosure may be sterilized by various sterilization methods, including, for example, sterile filtration or radiation. Sterile compositions for injection can be formulated according to conventional pharmaceutical practice as described in “Remington: The Science & Practice of Pharmacy,” 21st ed., Lippincott Williams & Wilkins, (2005).

In some embodiments, therapeutic compositions can be formulated for particular routes of administration, such as oral, nasal, pulmonary, topical (including buccal and sublingual), rectal, vaginal, and/or parenteral administration. The terms “parenteral administration” and “administered parenterally” as used herein refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection, and infusion. The inhibitors and other actives may be mixed under sterile conditions with a pharmaceutically acceptable carrier, and with any preservatives, buffers, or propellants which may be required (see, e.g., U.S. Pat. Nos. 7,378,110; 7,258,873; and 7,135,180; U.S. Patent Application Publication Nos. 2004/0042972 and 2004/0042971).

Also provided are kits, for example kits which include:
(i) a pharmaceutical composition containing an anti-CD73 antibody,
(ii) a pharmaceutical composition containing an anti-CD73 antibody, an anti-HER2 antibody, and optionally further a chemotherapy agent (e.g. a taxane, paclitaxel), or
(iii) a first pharmaceutical composition containing an anti-CD73 antibody, and a second pharmaceutical composition containing an anti-HER2 antibody, and optionally further a third pharmaceutical composition containing chemotherapy agent (e.g. a taxane, paclitaxel), or
(iv) a pharmaceutical composition containing an anti-CD73 antibody, and instructions to administer said anti-CD73 antibody with anti-HER2 antibody and optionally further with a chemotherapy agent (e.g. a taxane, paclitaxel), or
(v) a pharmaceutical composition containing an anti-HER2 antibody, and instructions to administer said PD-1 neutralizing agent with an anti-CD73 antibody and optionally further with a chemotherapy agent (e.g. a taxane, paclitaxel).

A pharmaceutical composition may optionally be specified as comprising a pharmaceutically-acceptable carrier. An anti-CD73 antibody, anti-HER2 antibody and chemotherapy agent may optionally be specified as being present in a therapeutically effective amount adapted for use in any of the methods herein. The kits optionally also can include instructions, e.g., comprising administration schedules, to allow a practitioner (e.g., a physician, nurse, or patient) to administer the composition contained therein to a patient having a cancer (e.g., a patient having a gastric adenocarcinoma or gastroesophageal junction adenocarcinoma which is HER2-positive, or a patient having a gastric adenocarcinoma or gastroesophageal junction adenocarcinoma whose tumor cells have a low or medium level of HER2 expression, or a patient having a gastric adenocarcinoma or gastroesophageal junction adenocarcinoma whose tumor cells have HER2 overexpression. In any embodiment, a kit optionally can include instructions to administer said CD73 antibody, anti-HER2 antibody and optionally chemotherapy agent according to a treatment regimens of the disclosure. The kit also can include a syringe.

Optionally, the kits include multiple packages of the single-dose pharmaceutical compositions each containing an effective amount of anti-CD73 antibody, anti-HER2 antibody and optionally chemotherapy agent, for a single administration in accordance with the methods provided above. Instruments or devices necessary for administering the pharmaceutical composition(s) also may be included in the kits. For instance, a kit may provide one or more pre-filled syringes containing an amount of the anti-CD73 antibody or anti-HER2 antibody.

In one embodiment, the present invention provides a kit for treating a cancer or a tumor in a human patient afflicted with cervical cancer, the kit comprising:
(a) a dose of an antiCD73 antibody comprising the H-CDR1, H-CDR2 and H-CDR3 domains of a heavy chain variable region having the sequence set forth in any of SEQ ID NOS: 42, and the L-CDR1, L-CDR2 and L-CDR3 domains of a light chain variable region having the sequence set forth in SEQ ID NO: 43; and/or
(b) a dose of an anti-HER2 antibody, optionally a dose of an antibody comprising the heavy and light chain CDR1, CDR2 and CDR3 domains of trastuzumab, optionally the H-CDR1, H-CDR2 and H-CDR3 domains of a heavy chain variable region having the sequence set forth in SEQ ID NO: 23, and the L-CDR1, L-CDR2 and L-CDR3 domains of a light chain variable region having the sequence set forth in SEQ ID NO: 24; and/or
(c) optionally, and a dose of a chemotherapy agent, optionally a dose of a taxane, optionally a dose of paclitaxel; and/or
(d) optionally, instructions for using said anti-CD73 antibody, said anti-HER antibody and/or said chemotherapy agent in any of the methods described herein.

In some embodiments, the anti-CD73 antibody comprises a heavy chain having the amino acid sequence of SEQ ID NO: 47 and a light chain having the amino acid sequence of SEQ ID NO: 48, the anti-HER2 antibody is trastuzumab or comprises a heavy chain having the amino acid sequence of SEQ ID NO: 25 and a light chain having the amino acid sequence of SEQ ID NO: 26, and the chemotherapy agent is paclitaxel. In some embodiments, the anti-CD73 antibody administered at a fixed dose (irrespective of body weight, for example a fixed dose of at least 900 mg, optionally 1500 mg or at least 1500 mg, or optionally 2400 mg or at least 2400 mg) every two weeks. In some embodiments, the anti-CD73 antibody administered at a fixed dose (e.g. a fixed dose of between 2000 and 3000 mg, for example at a fixed dose of 2100 mg, 2200 mg, 2300 mg, 2400 mg, 2500 mg, 2600 mg, 2700 mg, 2800 mg, 2900 mg or 3000 mg) every three weeks. In some embodiments, the anti-HER2 antibody is administered intravenously at dose of 3-6 mg/kg body weight, optionally 6-8 (e.g. 6 or 8) mg/kg body weight every three weeks (optionally wherein the first administration of anti-HER2 antibody is a loading dose of 8 mg/kg administered one week prior to the start of 3-weekly administration of 6 mg/kg), and the chemotherapy agent comprises paclitaxel administered at a fixed dose of 175 mg/m² body surface area. In some embodiments, the anti-HER2 antibody is administered intravenously at an initial dose of 4 mg/kg followed by subsequent weekly doses of 2 mg/kg. In some embodiments, the anti-HER2 antibody is administered subcutaneously at a fixed dose of 600 mg every three weeks.

In one embodiment, the anti-CD73 antibody and anti-HER2 antibody (and optionally chemotherapy agent) are administered at the following doses:
(a) (i) 900-3600 mg, optionally 1500-3600 mg, optionally 1500-2400 mg, optionally 900, 1500 or 2400 mg anti-CD73 antibody every 2 weeks, and (ii) 3-6 mg/kg (e.g., 5-6 or 6 mg/kg) body weight anti-HER2 antibody every 3 weeks, optionally wherein the first administration of anti-HER2 antibody is preceded by a loading dose (e.g., of 8 mg/kg body weight) anti-HER2 antibody administered one week earlier;
(b) (i) 900-3600 mg, optionally 1500-3600 mg, optionally 1500-2400 mg, optionally 900, 1500 or 2400 mg anti-CD73 antibody every 2 weeks, and (ii) a fixed dose of 600 mg anti-HER2 antibody administered subcutaneously (e.g. Herceptin™MSC) every 3 weeks;
(c) (i) 2000-3000 mg, optionally a fixed dose of 2100 mg, 2200 mg, 2300 mg, 2400 mg, 2500 mg, 2600 mg, 2700 mg, 2800 mg, 2900 mg or 3000 mg anti-CD73 antibody every three weeks, and (ii) 3-6 mg/kg (e.g., 5-6 or 6 mg/kg) body weight anti-HER2 antibody every 3 weeks, optionally wherein the first administration of anti-HER2 antibody is preceded by a loading dose (e.g., of 8 mg/kg body weight) anti-HER2 antibody administered one week earlier;
(d) (i) 2000-3000 mg, optionally a fixed dose of 2100 mg, 2200 mg, 2300 mg, 2400 mg, 2500 mg, 2600 mg, 2700 mg, 2800 mg, 2900 mg or 3000 mg anti-CD73 antibody every three weeks, and (ii) a fixed dose of 600 mg anti-HER2 antibody administered subcutaneously (e.g. Herceptin™MSC) every 3 weeks;
(e) (i) 900-3600 mg, optionally 1500-3600 m, optionally 1500-2400 mg, optionally 900, 1500 or 2400 mg anti-CD73 antibody every 2 weeks, (ii) 3-6 mg/kg (e.g., 5-6 or 6 mg/kg) body weight anti-HER2 antibody every 3 weeks, optionally wherein the first administration of anti-HER2 antibody is preceded by a loading dose (e.g., of 8 mg/kg body weight) anti-HER2 antibody administered one week earlier, and (iii) 175 mg/m² body surface area paclitaxel administered every three weeks;
(f) (i) 1500 mg anti-CD73 antibody every 2 weeks, and (ii) 6 mg/kg body weight anti-HER2 antibody every 3 weeks, optionally wherein the first administration of anti-HER2 antibody is preceded by a loading dose (e.g., of 8 mg/kg body weight) anti-HER2 antibody administered one week earlier;
(g) (i) 1500 mg anti-CD73 antibody every 2 weeks, (ii) 6 mg/kg body weight anti-HER2 antibody every 3 weeks, optionally wherein the first administration of anti-HER2 antibody is preceded by a loading dose (e.g., of 8 mg/kg body weight) anti-HER2 antibody administered one week earlier, and (iii) 175 mg/m² body surface area paclitaxel administered every three weeks;
(h) (i) 2400 mg anti-CD73 antibody every 2 weeks, and (ii) 6 mg/kg body weight anti-HER2 antibody every 3 weeks, optionally wherein the first administration of anti-HER2 antibody is preceded by a loading dose (e.g., of 8 mg/kg body weight) anti-HER2 antibody administered one week earlier; or
(i) (i) 2400 mg anti-CD73 antibody every 2 weeks, (ii) 6 mg/kg body weight anti-HER2 antibody every 3 weeks, optionally wherein the first administration of anti-HER2 antibody is preceded by a loading dose (e.g., of 8 mg/kg body weight) anti-HER2 antibody administered one week earlier, and (iii) 175 mg/m² body surface area paclitaxel administered every three weeks.

Without limiting the disclosure, a number of embodiments of the disclosure are described herein for purpose of illustration.

### EXAMPLES

### Methods

### Immunostaining in Human Gastric Cancer Samples

CD73 immunostainings were performed on a Ventana Benchmark Ultra. Briefly, 5 μm-thick FFPE sections were dewaxed and antigen retrieval performed using CC1 for 64 minutes at 100° C. Then, anti-CD73 antibody diluted to 1 μg/mL in the discovery antibody diluent was applied for 1 h at 37° C., and revealed using ULTRAVIEW-RT. Formalin-fixed hCD73 cell pellets were used as negative and positive controls in each IHC run. Immunostaining analysis was performed on tumor cells, stromal cells and immune cells, recording the percentage of cells expressing CD73. These percentages were then expressed as proportion score, as follows: 0=no positive cells; 1=<10% positive cells; 2=between 10 and 50% positive cells; 3=between 51 and 80% positive cells; 4=>80% positive cells.

A semi-quantitative intensity of CD73 staining on tumor cells and stromal cells was also recorded as 0, 1+, 2+ and 3+. For CD73 on tumor cells an H-score was also calculated. The percentage of cells at each staining intensity level was calculated, and finally, an H-score was assigned using the following formula: [1×(% cells 1+)+2×(% cells 2+)+3×(% cells 3+)].

The final score, ranging from 0 to 300, gives more relative weight to higher-intensity membrane staining in a given tumor sample. CD73 expression was also recorded on endothelial cells as staining positive control.

Cloning, Production and Purification of Recombinant huCD73

### Molecular Biology

The huCD73 protein was cloned from MIAPACA-2 cDNA using the following primers TACGACTCACAAGCTTGCCGCCACCATGTGTCCCCGAGCCGCGCG (SEQ ID NO: 20) (Forward) and CCGCCCCGACTCTAGAtcaGTGATGGTGATGATGGTGcttgatccgaccttcaactg SEQ ID NO: 21) (Reverse). The purified PCR product was then cloned into an expression vector using the InFusion cloning system. A 6xHis tag was added in the C-terminal part of the protein for the purification step.

Amino acid sequence of the cloned huCD73:

(SEQ ID NO: 22)
MCPRAARAPATLLLALGAVLWPAAGAWELTILHTNDVHSRLEQTSEDSS
KCVNASRCMGGVARLFTKVQQIRRAEPNVLLLDAGDQYQGTIWFTVYKG
AEVAHEMNALRYDAMALGNHEFDNGVEGLIEPLLKEAKEPILSANIKAK
GPLASQISGLYLPYKVLPVGDEVVGIVGYTSKETPFLSNPGTNLVFEDE
ITALQPEVDKLKTLNVNKIIALGHSGFEMDKLIAQKVRGVDVVVGGHSN
TFLYTGNPPSKEVPAGKYPFIVTSDDGRKVPVVQAYAFGKYLGYLKIEF
DERGNVISSHGNPILLNSSIPEDPSIKADINKWRIKLDNYSTQELGKTI
VYLDGSSQSCRFRECNMGNLICDAMINNNLRHTDEMFWNHVSMCILNGG
GIRSPIDERNNGTITWENLAAVLPFGGTFDLVQLKGSTLKKAFEHSVHR
YGQSTGEFLQVGGIHVVYDLSRKPGDRVVKLDVLCTKCRVPSYDPLKMD
EVYKVILPNFLANGGDGFQMIKDELLRHDSGDQDINVVSTYISKMKVIY
PAVEGRIKHHHHHH.

Expression and purification of the huCD73 proteins

After validation of the sequence cloned, cells were nucleofected and the producing pool was then sub-cloned to obtain a cell clone producing the huCD73 protein. Supernatant from the huCD73 clone grown in roller was harvested and purified using Ni-NTA column and eluted using 250 mM imidazole. The purified proteins were then loaded onto a S200 size exclusion chromatography column. The purified protein corresponding to a dimer was formulated in a Tris 20 mM pH 7.5, NaCl 120 mM and CaCI2 4 mM buffer for enzyme activity assays, while formulation buffer is supplemented with 20% glycerol.

### SPR Analysis to Assess Ab KD on Recombinant CD73 Protein

SPR measurements were performed on a Biacore™ T200 apparatus at 25° C. Protein-A (GE Healthcare) was immobilized on a Sensor Chip CM5 (GE Healthcare). The chip surface was activated with EDC/NHS (N-ethyl-N′-(3-dimethylaminopropyl) carbodiimide hydrochloride and N-hydroxysuccinimide (Biacore™, GE Healthcare). Protein-A was diluted to 10 μg/mL in coupling buffer (10 mM acetate, pH 5.6) and injected until the appropriate immobilization level was reached (i.e. 2000 RU). Deactivation of the remaining activated groups was performed using 100 mM ethanolamine pH 8 (Biacore™, GE Healthcare). Affinity study was carried out according to a standard Capture-Kinetic protocol recommended by the manufacturer (Biacore ™, GE Healthcare kinetic wizard). Serial dilutions of human recombinant soluble CD73 protein, ranging from 1.23 to 300 nM were sequentially injected over the captured anti-CD73 antibodies and allowed to dissociate for 10 min before regeneration. The entire sensorgram sets were fitted using the 1:1 kinetic binding model. Bivalent affinities and kinetic association and dissociation rate constants are calculated.

### Flow Cytometry Analysis to Assess Anti-CD73 Recognition by Antibodies

10⁵ MDA-MB-231 cells resuspended in FCSB were distributed into round-bottom 96W-microplates (50 μL per well). A dose range of anti-CD73 mAbs was added to the plates and cells were incubated for 1 h at +5+3° C. Cells were washed three times in FCSB by spinning plates at 400 g for 3 min at 4° C. PE-conjugated Goat F(ab′)2 Anti-Human IgG (H+L) secondary Ab diluted in FCSB was added to the cells and plates were incubated for 30 additional minutes at +5+3° C. Cells were washed three times as described above and analyzed on a flow cytometer.

Median of fluorescence intensity vs. mAb concentration was plotted on graphs and EC₅₀ was calculated using GraphPad Prism™ program.

### In Vitro Enzymatic Assay With Recombinant Human or Cynomolgus CD73

Briefly, recombinant human or cynomolgus CD73 was incubated in white 96W flat-bottom microplates in the presence of a dose-range of anti-CD73 or isotype control mAbs. Plates were incubated for 1h at +37+1° C. ATP (12.5 μM) and AMP (125 μM) were added to each well and plates were incubated at +37+1° C. for 30 additional minutes. Luciferase/luciferin-containing CellTiter-Glo™ (Promega) was added into wells, plates were incubated for 5 minutes at RT in the dark and emitted light was measured using an Enspire™ apparatus (Perkin Elmer).

### Conditions:

ATP+AMP: maximal luciferase inhibition (100%)
CD73+ATP+AMP: no luciferase inhibition (0%)

The residual enzyme activity vs. anti-CD73 Ab concentration was plotted on graphs using GraphPad Prism7™ software.

### T Cell Proliferation Assay

Peripheral blood from healthy donors was obtained and mononuclear cells were isolated on a Ficoll gradient. Lymphocytes were further enriched on a 52% Percoll™ gradient (cell pellets) and stained with a 2 μM CellTrace™ Violet (Thermofisher). 5×10⁴ to 1×10⁵ of stained cells were distributed in 96w round-bottom plates, incubated for 1h at 37° C. with anti-CD73 Abs and activated for 3 to 5 days by addition of anti-CD3/anti-CD28-coated beads. Inhibition of T cell proliferation was achieved by addition of 200 μM AMP. T cell proliferation and the ability of Abs to block the immune suppressive effect of AMP were assessed by flow cytometry by quantifying the dye dilution on proliferating T cells. Percentage of proliferating T cells vs. anti-CD73 Ab concentration was plotted on graphs using GraphPad Prism™ software. Some experiments were done on whole PBMC from healthy donors or cancer patients; the protocol was as described above except that T cell suppression was achieved by addition of 0.5 to 1 mM ATP.

In order to compare donors or patients, T cell proliferation was normalized using the following formula:

$$\frac{( Activated cells + AT ( M ) P + Ab ) - ( Activated cells + AT ( M ) P )}{( Activated cells ) - ( Activated cells + AT ( M ) P )} \times 100$$

### Allogeneic Mixed Lymphocyte Reaction (MLR) Assay

Mononuclear cells from healthy donors were isolated on a Ficoll gradient and monocytes were purified by immunomagnetic selection using CD14 microbeads (Miltenyi Biotec). Monocytes were differentiated into dendritic cells (MoDC) by 5-7 days of culture in presence of GM-CSF (400 ng/ml) and IL-4 (20 ng/ml). The day of DC recovery, CD4+ T cells from allogeneic donors were purified by immunomagnetic depletion of non-CD4+ T cells (Miltenyi Biotec) and stained with Cell Trace™ Violet. DC (10⁴ cells/well) and T cells (5×10⁴ cells/well) were mixed in 96W round bottom microplates in presence of a dose-ranges of anti-huCD73 Abs and a fixed dose of ATP. T cell proliferation and Ab ability to reverse ATP-mediated suppression was assessed as described for T cell proliferation assay.

### Example 1

### CD73 and HER2 Expression in Gastric Cancer

For this study in a gastric cancer cohort the following tumor samples were investigated: a total of 60 surgical samples from primary tumor at diagnosis, of which 10 surgical samples were from primary tumors and the 10 surgical samples were from related metastasis at first relapse, 20 were biopsies at diagnosis, and 20 were surgical samples of the corresponding tumor after neoadjuvant chemotherapy (Epirubicin, Cisplatin & 5-FU).

At diagnosis (before any treatment) CD73 was found to be expressed on tumor cells (TC) in 83% of patients (n=50/60), with a percentage of tumor cells expressing CD73 varying from 5 to 80% across patients. Staining on tumor cells was mainly polarized at the apical level (luminal part) of the cells. 95% of patients had CD73-expressing stromal cells (SC) with a percentage of stromal cells expressing CD73 ranging from 1 to 50%. Only 5% of patients had CD73-expressing intratumoral immune cells (n=3/60) whereas 78% of patients had CD73-expressing immune cells in the stroma, with a range of percentage from 1 to 20% of CD73+.

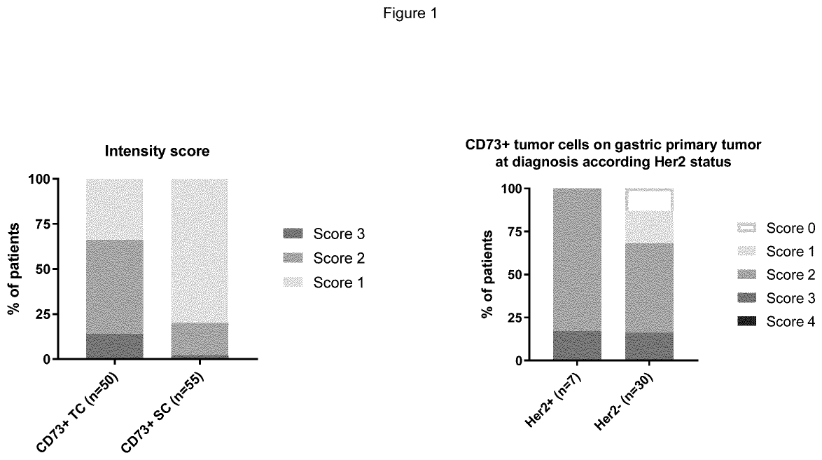

The H-score of CD73 on tumor cells varied from 0 to 210 with a median score of 37.5 (FIG. 1, left panel).

### CD73 Expression According to Her2 Status

CD73 expression on tumor cells and stromal cells was analyzed according to Her2 status. Her2 scoring evaluated by IHC staining was indicated as 1+, 2+, 3+ and the amplification of Her2 gene was evaluated by FISH. For the data analysis, and in accordance with the pathologist, patients were considered Her2 positive only when their “Her2 status for analysis (−/+)” was recorded as 3+ by IHC.

Analysis showed that all Her2+ patients had CD73-expressing tumor cells (FIG. 1, right panel). There was a lower number of Her2+ patients (n=7) compared to Her2− (n=30), however Her2+ patients had a significantly higher percentage of CD73+ tumor cells (FIG. 1, right panel). CD73-expressing stromal cells did not vary with Her2 status.

In conclusion, patient stratification according to Her2 status highlighted that all Her2+ patients had CD73-expressing tumor, and that Her2+ patients had a significantly higher percentage of CD73+ tumor cells than Her2−. All patients had CD73+ stromal cells irrespective of Her2 status.

### Comparison of CD73 Expression in Gastric Tumor Pre- and Post-neoadjuvant Chemotherapy

CD73 expression was compared between biopsies from primary tumor before neoadjuvant chemotherapy (CT) and tumor resection after neoadjuvant chemotherapy (Epirubicin, Cisplatin & 5-FU).

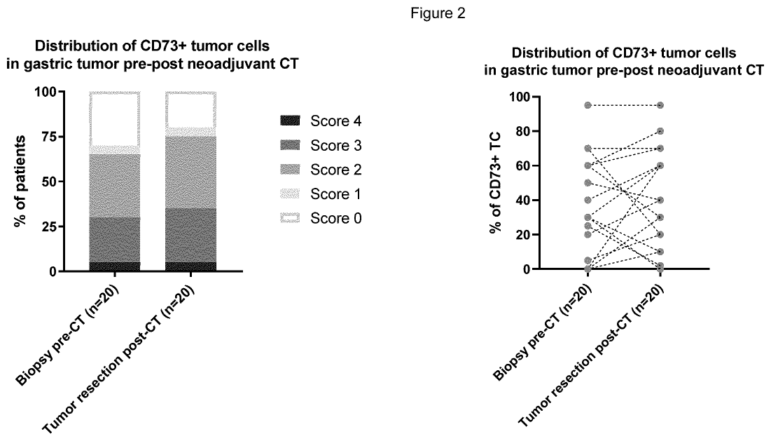

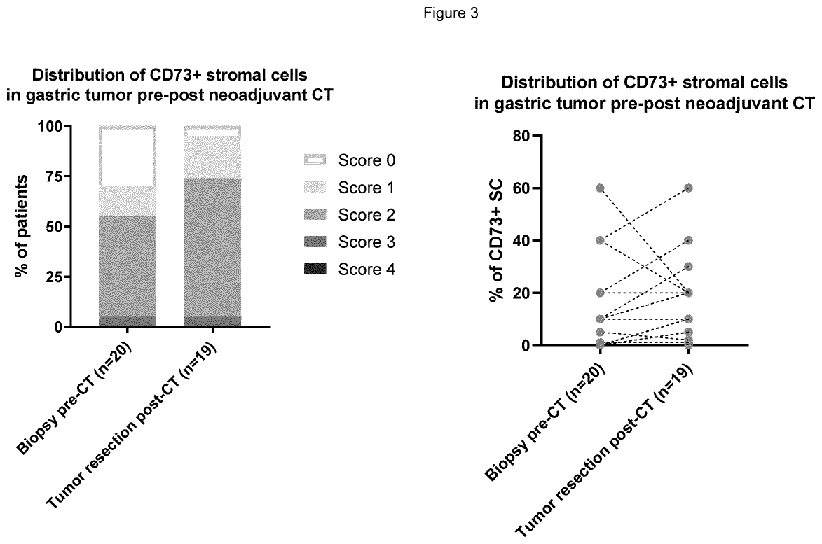

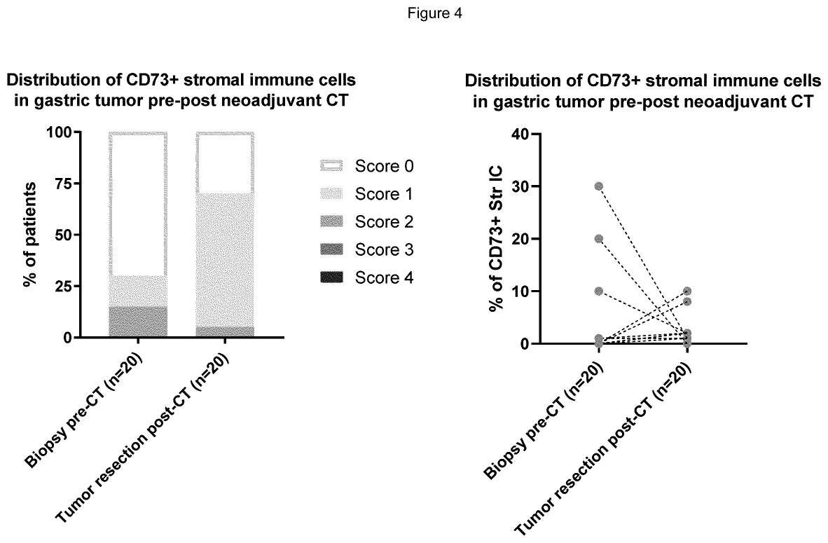

Neoadjuvant CT did not significantly modify CD73-expressing tumor cells either in the percentage of patients expressing CD73 (FIG. 2, left panel) or in the percentage of CD73+ tumor cells (FIG. 2, right panel). However, neoadjuvant CT increased the percentage of patients with CD73+ stromal cells from 60% pre-CT (n=12/20) to 95% post-CT (n=19/20) (FIG. 3, left panel). The percentage of CD73-expressing stromal cells was not significantly different pre- and post-CT (FIG. 3, right panel). CT increased the percentage of patients with CD73+ stromal immune cells from 30% (n=6/20) to 70% (n=14/20) (FIG. 4, left panel). A closer analysis of the percentage of CD73+ stromal immune cells indicated that CT tended to decrease this percentage when initially high whereas it induced CD73 on immune cells when initially negative (FIG. 4, right panel).

### Example 2

### Design of Humanized Anti-CD73 Antibodies

Humanized anti-CD73 antibodies were prepared as described in co-pending PCT application no. PCT/EP2020/060955 filed 20 Apr. 2020, the disclosure of which is incorporated herein by reference, based on murine antibodies that were observed to bind to CD73 in an intra-dimer binding mode in a 1:1 stoichiometry between an intact full-length antibody and a CD73 dimer.

Briefly, antibodies were modified by the introduction into the VH of heavy chain frameworks (FR1, FR2, FR3) from the human subgroup IGHV1-3*01 together with IGHJ4*01 (FR4), and the introduction into the VL of light chain frameworks (FR1, FR2, FR3) from the human subgroup IGKV1-33*01, together with IGKJ2*01 (FR4).

For a first set of humanized antibodies, four different humanized heavy chains and four different humanized light chains were designed. A first heavy chain variant (H1) having the amino acid sequence shown in SEQ ID NO: 29 had a V2I and a T73K substitution. A second heavy chain (H2) variant having the amino acid sequence shown in SEQ ID NO: 30 had the substitutions V2I, T28A, R71V and T73K. A third heavy chain variant (H3) having the amino acid sequence shown in SEQ ID NO: 31 had the substitutions V2I, T28A, M48I, R66K, V67A, R71V and T73K. A fourth heavy chain variant (H4) having the amino acid sequence shown in SEQ ID NO: 32 had the substitutions V2I, T28A, T30A, M48I, R66K, V67A, I69L, R71V and T73K. Numbering of substitutions is according to Kabat.

A first light chain variant (L1) having the amino acid sequence shown in SEQ ID NO: 33 had the substitution S67Y. A second light chain (L2) variant having the amino acid sequence shown in SEQ ID NO: 34 had the substitutions S60D and S67Y. A third light chain variant (L3) having the amino acid sequence shown in SEQ ID NO: 35 had the substitutions I2V, S60D and S67Y. A fourth light chain variant (L4) having the amino acid sequence shown in SEQ ID NO: 36 had the substitutions I2V, S60D, S67Y and Y87F. Numbering of substitutions is according to Kabat.

The amino acid sequences of respective heavy (“H” chains in Table 1) and light (“L” chains in Table 1) chain variable regions are shown in the Table 1 below.

TABLE 1

| | SEQ | Sequence |
|---|---|---|
| Chain | ID NO | (amino acid substitutions in bold, Kabat CDRs underlined) |
| H1 | 29 | QIQLVQSGAEVKKPGASVKVSCKASGYTFTSYNMYWVRQAPGQRLEWMGYIDPYNGGS |
| | | SYNLTFKGRVTITRDKSASTAYMELSSLRSEDTAVYYCARGYGNYKAWFAYWGQGTLV |
| | | TVSS |
| H2 | 30 | QIQLVQSGAEVKKPGASVKVSCKASGYAFTSYNMYWVRQAPGQSLEWMGYIDPYNGGS |
| | | SYNLTFKGRVTITVDKSASTAYMELSSLRSEDTAVYYCARGYGNYKAWFAYWGQGTLV |
| | | TVSS |
| H3 | 31 | QIQLVQSGAEVKKPGASVKVSCKASGYAFTSYNMYWVRQAPGQSLEWIGYIDPYNGGS |
| | | SYNLTFKGKATITVDKSASTAYMELSSLRSEDTAVYYCARGYGNYKAWFAYWGQGTLV |
| | | TVSS |
| H4 | 32 | QIQLVQSGAEVKKPGASVKVSCKASGYAFASYNMYWVRQAPGQSLEWIGYIDPYNGGS |
| | | SYNLTFKGKATLTVDKSASTAYMELSSLRSEDTAVYYCARGYGNYKAWFAYWGQGTLV |
| | | TVSS |
| L1 | 33 | DIQMTQSPSSLSASVGDRVTITCKASQSVSNDVAWYQQKPGKAPKLLIYYASTRYTGV |
| | | PSRFSGSGYGTDFTFTISSLQPEDIATYYCQQDYSSLTFGQGTKLEIK |
| L2 | 34 | DIQMTQSPSSLSASVGDRVTITCKASQSVSNDVAWYQQKPGKAPKLLIYYASTRYTGV |
| | | PDRFSGSGYGTDFTFTISSLQPEDIATYYCQQDYSSLTFGQGTKLEIK |
| L3 | 35 | DVQMTQSPSSLSASVGDRVTITCKASQSVSNDVAWYQQKPGKAPKLLIYYASTRYTGV |
| | | PDRFSGSGYGTDFTFTISSLQPEDIATYYCQQDYSSLTFGQGTKLEIK |
| L4 | 36 | DVQMTQSPSSLSASVGDRVTITCKASQSVSNDVAWYQQKPGKAPKLLIYYASTRYTGV |
| | | PDRFSGSGYGTDFTFTISSLQPEDIATYFCQQDYSSLTFGQGTKLEIK |

The antibodies having the heavy and light chain combinations shown in Table 2 below were produced.

TABLE 2

Light chains

| | | L0 | L1 | L2 | L3 | L4 |
|---|---|---|---|---|---|---|
| Heavy | H0 | H0L0 | H0L1 | H0L2 | H0L3 | H0L4 |
| chains | H1 | H1L0 | H1L1 | H1L2 | H1L3 | H1L4 |
| | H2 | H2L0 | H2L1 | H2L2 | H2L3 | H2L4 |
| | H3 | H3L0 | H3L1 | H3L2 | H3L3 | H3L4 |
| | H4 | H4L0 | H4L1 | H4L2 | H4L3 | H4L4 |

Surprisingly, studies showed that there was no direct correlation between flow cytometry titration and potency in inhibition of the enzymatic activity of CD73. Interestingly, while H1, H2, H3 and H4 variants were essentially indistinguishable based on binding affinity to CD73 recombinant protein in SPR assays and in flow cytometry on CD73-expressing cells, the H2 and H3 variants were less potent in the functional assays than H4 variants in the ability to restore T cell proliferation. One possibility is that the mutations introduced into the H2 and H3 frameworks which were detrimental to potency in inhibiting CD73 (but were not detrimental to binding affinity for CD73) were compensated by mutations introduced into the H4 variant frameworks. Consequently, new variants with substitutions in heavy chain framework and/or CDR residues were designed. Based on these observations, the substitutions made in the H2, H3 and H4 chains were classified as either residues that are believed to be critical for function, residues likely not to affection function, residues that negatively impact function, and residues that restore function.

A new heavy chain variant, referred to as “H4+”, having the amino acid sequence shown below (SEQ ID NO: 37; amino acid substitutions in bold, Kabat CDRs underlined), was designed and were combined with L1, L2, L3 and L4 light chains (of SEQ ID NOS: 33, 34, 35 and 36, respectively) to generate four new antibodies, H4+L1, H4+L2, H4+L3 and H4+L4. H4+ VH:

(SEQ ID NO: 37)
QIQLVQSGAEVKKPGASVKVSCKASGYTFASYNMYWVRQAPGQRLEWIG
YIDPYNGGSSYNLTFKGRVTLTRDKSASTAYMELSSLRSEDTAVYYCAR
GYGNYKAWFAYWGQGTLVTVSS.

The amino acid sequence of the full H4+ heavy chain comprising a human IgG1 Fc domain with L234A/L235E/G237A/A330S/P331S substitutions is shown below:

(SEQ ID NO: 38)
QIQLVQSGAEVKKPGASVKVSCKASGYTFASYNMYWVRQAPGQRLEWIG
YIDPYNGGSSYNLTFKGRVTLTRDKSASTAYMELSSLRSEDTAVYYCAR
GYGNYKAWFAYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKRVEPKSCDKTHTCPPCPAPEAEGAPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPSSIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK.

The H4+L1 antibody contained a L1 light chain comprising a human Ckappa domain, the full sequence of which is shown below:

(SEQ ID NO: 39)
DIQMTQSPSSLSASVGDRVTITCKASQSVSNDVAWYQQKPGKAPKLLIY
YASTRYTGVPSRFSGSGYGTDFTFTISSLQPEDIATYYCQQDYSSLTFG
QGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC.

In order to investigate how the same framework substitutions would impact an antibody having differences in its heavy and light chain CDRs, a further series of four antibodies was produced having a common heavy chain and one of four different light chains. The common heavy chain designated “2H4+” had a glutamine at Kabat position 59 (Q59) in HCDR2 (i.e. L59Q substitution, compared to the H4+ chain), a lysine at Kabat residue 60 (K60) in HCDR2 (i.e. a T60K substitution, compared to the H4+ chain), and an asparagine at Kabat position 97 (N97) in HCDR3 (i.e. a G97N substitution, compared to the H4+ chain). The four light chains designated 2L1, 2L2, 2L3 and 2L4 had the same framework residues as L1, L2, L3 and L4 chains, respectively, and differed in their CDRs from L1-L4 chains by presence of a threonine at Kabat position 30 (T30) in LCDR1 (i.e. a S30T substitution, compared to the L1-L4 chains), and an asparagine at Kabat residue 53 (N53) in LCDR2 (i.e. a T53N substitution, compared to the L1-L4 chains).

The resulting four new antibodies were designated 2H4+2L1, 2H4+2L2, 2H4+2L3 and 2H4+2L4 (see Table 4 below for combinations of heavy and light chains, and Table 3 below for amino acid sequences of the individual chains). A parental antibody sharing the CDRs in murine frameworks was produced with heavy and light chain variable regions designated 2HP and 2LP (see Table 3, below).

TABLE 3

| | SEQ | Variable region amino acid sequence |
|---|---|---|
| Chain | ID NO | (amino acid substitutions in bold, Kabat CDRs underlined) |
| 2HP | 40 | EIQLQQSGPELVKPGASVKVSCKASGYAFTSYNMYWVKQSHGKRLEWIGYIDPYNG |
| | | GSSYNQKFKGKATLTVDKSSSTAYMHLNNLTSEDSAVYYCARGYNNYKAWFAYWGQ |
| | | GTLVTVSA |
| 2LP | 41 | DVVMTQTPKFLLVSAGDRVTITCKASQSVTNDVAWYQQKPGQSPKLLIYYASNRYT |
| | | GVPDRFTGSGYGTDFTFTISTMQAEDLAVYFCQQDYSSLTFGAGTKLELK |
| 2H4+ | 42 | QIQLVQSGAEVKKPGASVKVSCKASGYTFASYNMYWVRQAPGQRLEWIGYIDPYNG |
| | | GSSYNQKFKGRVTLTRDKSASTAYMELSSLRSEDTAVYYCARGYNNYKAWFAYWGQ |
| | | GTLVTVSS |
| 2L1 | 43 | DIQMTQSPSSLSASVGDRVTITCKASQSVTNDVAWYQQKPGKAPKLLIYYASNRYT |
| | | GVPSRFSGSGYGTDFTFTISSLQPEDIATYYCQQDYSSLTFGQGTKLEIK |
| 2L2 | 44 | DIQMTQSPSSLSASVGDRVTITCKASQSVTNDVAWYQQKPGKAPKLLIYYASNRYT |
| | | GVPDRFSGSGYGTDFTFTISSLQPEDIATYYCQQDYSSLTFGQGTKLEIK |
| 2L3 | 45 | DVQMTQSPSSLSASVGDRVTITCKASQSVTNDVAWYQQKPGKAPKLLIYYASNRYT |
| | | GVPDRFSGSGYGTDFTFTISSLQPEDIATYYCQQDYSSLTFGQGTKLEIK |
| 2L4 | 46 | DVQMTQSPSSLSASVGDRVTITCKASQSVTNDVAWYQQKPGKAPKLLIYYASNRYT |
| | | GVPDRFSGSGYGTDFTFTISSLQPEDIATYFCQQDYSSLTFGQGTKLEIK |

TABLE 4

| mAb reference | VH | VL |
|---|---|---|
| 2H4 + L1 | 2H4 + | 2L1 |
| | (SEQ ID NO: 42) | (SEQ ID NO: 43) |
| 2H4 + L2 | 2H4 + | 2L2 |
| | (SEQ ID NO: 42) | (SEQ ID NO: 44) |
| 2H4 + L3 | 2H4 + | 2L3 |
| | (SEQ ID NO: 42) | (SEQ ID NO: 45) |
| 2H4 + L4 | 2H4 + | 2L4 |
| | (SEQ ID NO: 42) | SEQ ID NO: 46 |
| 2HP2LP | 2HP | 2LP |
| | (SEQ ID NO: 40) | (SEQ ID NO: 41) |

All antibodies were produced as human IgG1 isotype with L234A/L235E/G237A/A330S/P331S substitutions to reduce Fc receptor binding.

The amino acid sequence of the full 2H4+ heavy chain comprising a human IgG1 Fc domain with L234A/L235E/G237A/A330S/P331S substitutions is shown below:

(SEQ ID NO: 47)
QIQLVQSGAEVKKPGASVKVSCKASGYTFASYNMYWVRQAPGQRLEWIG
YIDPYNGGSSYNQKFKGRVTLTRDKSASTAYMELSSLRSEDTAVYYCAR
GYNNYKAWFAYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKRVEPKSCDKTHTCPPCPAPEAEGAPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPSSIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK.

The amino acid sequence of the full 2L1 light chain of the antibody comprising a human Ckappa domain is shown below:

(SEQ ID NO: 48)
DIQMTQSPSSLSASVGDRVTITCKASQSVTNDVAWYQQKPGKAPKLLIY
YASNRYTGVPSRFSGSGYGTDFTFTISSLQPEDIATYYCQQDYSSLTFG
QGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC.

### Example 3

### Study of New Variants in Vitro Enzymatic Assay With Recombinant CD73 Protein

The efficacy of the humanized variants were compared to the parental antibody to inhibit the enzymatic activity of the recombinant CD73 protein. Briefly, high levels of luminescence was measured when ATP and CTG substrates were mixed. When AMP was added to the reaction mix, the CTG reaction was inhibited resulting in a decrease of luminescence. In the presence of recombinant human CD73 protein that hydrolyzes AMP, the level of luminescence was restored.

All of the new humanized variants produced in Example 2 potently inhibited the activity of the CD73 protein.

The H4+Lx antibodies were as efficient as their parental counterpart. Furthermore, all of the 2H4+2Lx (2H4+L1, 2H4+L2, 2H4+L3 and 2H4+L4) variants showed an increase in potency in CD73 inhibition compared to the H4+Lx variants. The 2H4+2Lx variants inhibited the activity of CD73 with an efficacy similar to that of the parental 2HP2LP antibody.

An additional experiment was performed with different concentrations of the CD73 protein (50, 100, 200, 400 ng/ml). The aim of this experiment was to study the ability of the humanized antibodies to block the activity of high amounts of CD73 protein. Again, the 2H4+2Lx variants were as potent as the parental 2HP2LP antibody.

### Example 4

### Study of new Variants for Efficacy on Cynomolgus CD73 Protein

An experiment on recombinant rec cyCD73 protein was performed with 2H1Lx and 2H4+2Lx variants. Experimental conditions were the same as in experiments on human protein except for the concentration of cyCD73 protein that was used (400 ng/mL).

The efficacy of the humanized variants to block enzymatic activity of cyCD73 protein was the same as that of the parental (chimeric) antibody.

### Example 5

### Study of new Variants in T Cell Proliferation Assay

Humanized variants of the different antibodies were tested for their ability to restore T cell proliferation inhibited by AMP. Briefly, Peripheral blood from healthy donors (HD) was obtained from EFS (anticoagulant: citrate), and mononuclear cells were isolated on a Ficoll gradient. Lymphocytes were further enriched on a 52% Percoll gradient prepared in PBS solution. Cells were stained with a Cell Trace dye. Stained cells were distributed in 96 round-bottom plates, incubated for 1 h at +37÷1° C. with anti-huCD73 Abs and activated for 3 to 5 days by addition of anti-CD3/anti-CD28 beads (bead to cell ratio=1:4). Inhibition of T cell proliferation was achieved by addition of AMP (800 μM). T cell proliferation and ability of Abs to block the immune suppressive effect of AMP were assessed by flow cytometry by quantifying the dye dilution on proliferating T cells.

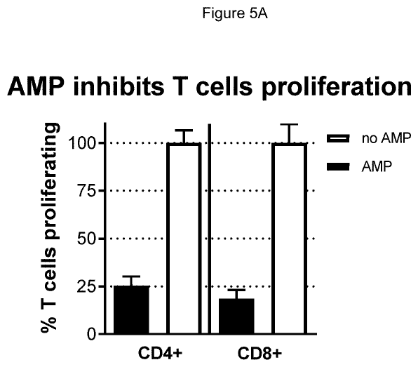

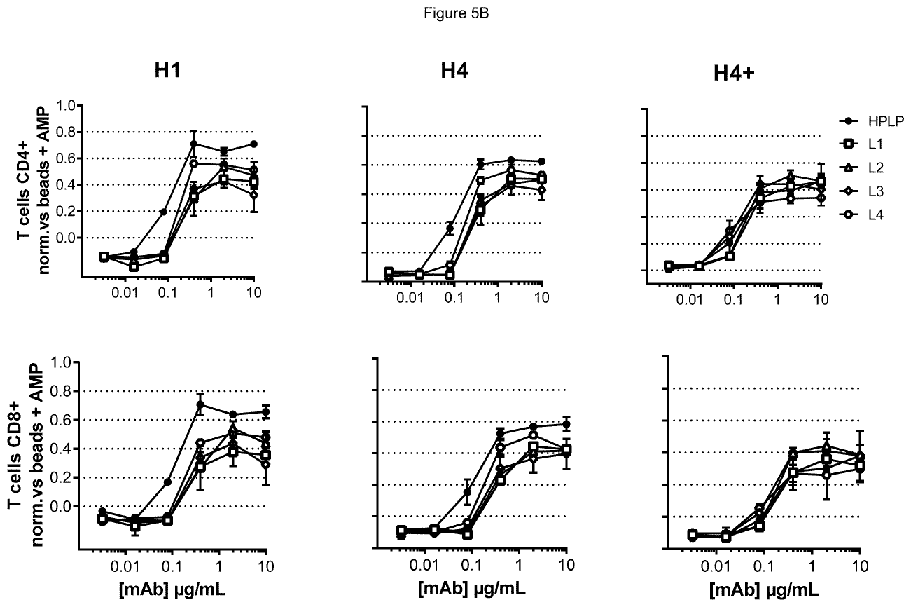

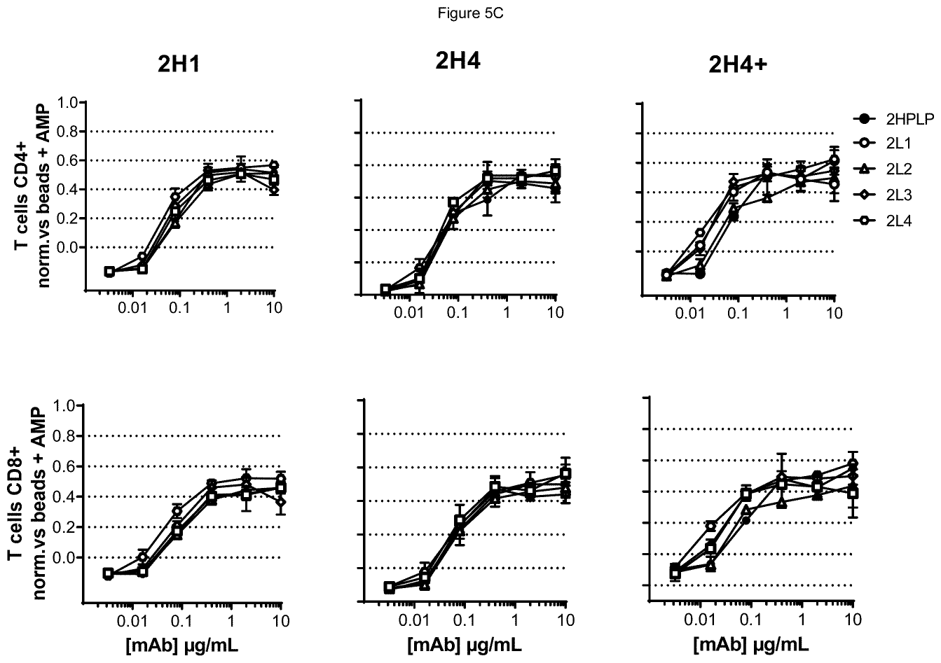

FIG. 5A shows the inhibitory effect of AMP on T cell proliferation. All humanized variants potently blocked inhibitory effect of AMP on T cell proliferation (FIGS. 5B and C). While most humanized variants generally appear to be as efficient as their chimeric parental counterpart in reversing AMP-mediated T cell suppression, the 2H4+2Lx variants were the most potent among all variants, and surprisingly they were even more potent than the parental 2HP2LP antibody to block the suppressive effects of AMP.

### Example 6

### Comparative Study of New Variants in T cell Proliferation Assay in two Human Donors

According to results previously obtained, the 2H4+2Lx humanized variants were further characterized in further series of T cell proliferation experiments.

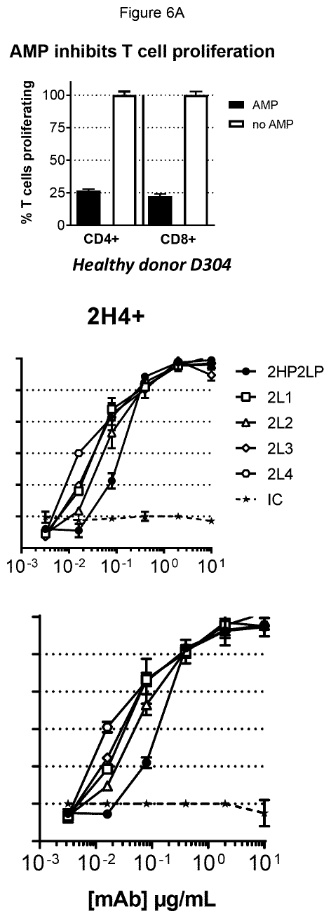

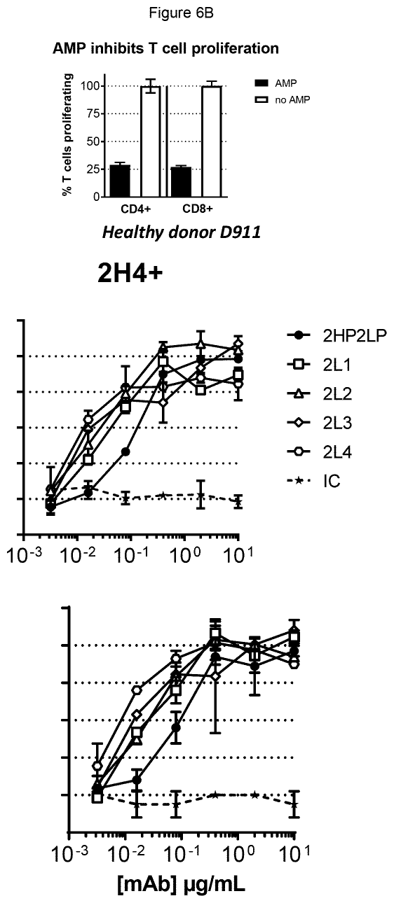

FIGS. 6A and 6B shows results obtained in a T cell proliferation assay using cells from two healthy donors, respectively. As observed previously, 2H4+2Lx variants were each more potent than the parental 2HP2LP to restore T cell proliferation (FIG. 6A and B, right panels). No clear differences among the different 2H4+2Lx variants were observed. Thus, once again, the 2H4+2Lx variants were more potent than the parental 2HP2LP antibody to block the suppressive effects of AMP.

Taken together, the results obtained on T cell proliferation assay indicate that the humanized variants having the frameworks of the H4++ chains are the most potent antibody variants, and that the 2H4+2Lx variants overall have the greatest potency amongst all the antibodies.

### Example 7

### Study of new Variants in Allogeneic Mixed Lymphocyte Reaction (MLR) Assay

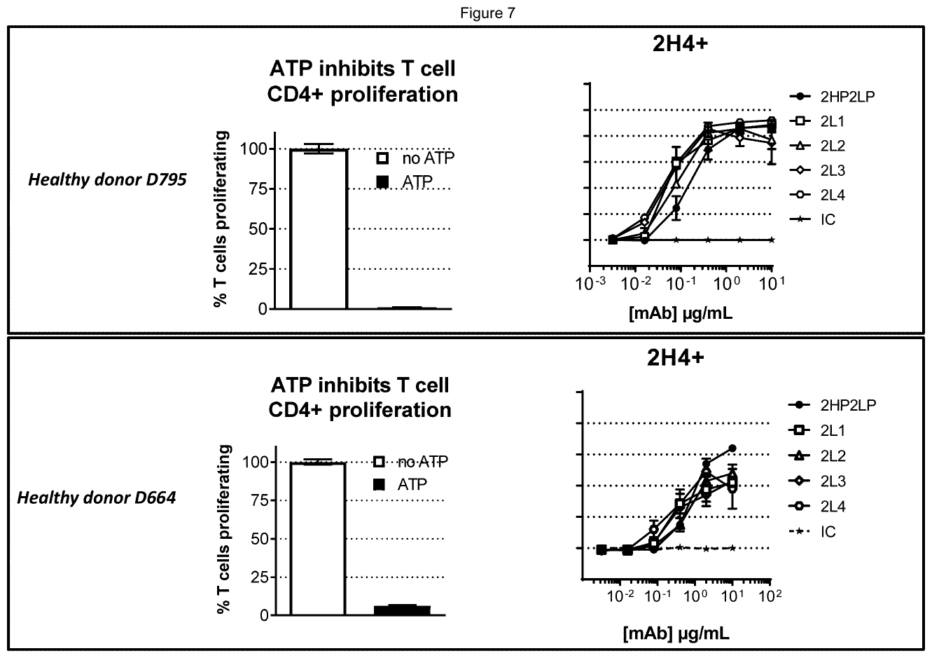

The 2H4+2Lx antibody variant was tested in an allogenic MLR in order to confirm the results obtained previously in T cell proliferation assays. Briefly, PBMC from healthy human donors were enriched through a Ficoll density gradient and monocytes were purified by positive immunomagnetic selection (Miltenyi Biotec). Monocytes were differentiated into dendritic cells (DC) by 5-7 days of culture in presence of GM-CSF (400 ng/mL) and IL-4 (10 ng/ml). The day of DC recovery, CD4+ T cells from allogeneic donors were purified by immunomagnetic depletion of non-CD4+ T cells (Miltenyi Biotec) and stained with Cell Trace dye. DC and T cells were mixed in 96W round bottom microplates in presence of a dose range of anti-huCD73 mAbs and a fixed dose of 100UM ATP. T cell proliferation and mAb ability to reverse ATP-mediated suppression was then assessed after 6 days of co-culture. T cell proliferation was inhibited by adding 100 μM of ATP (which is degraded into ADP and AMP by CD39), shown in FIG. 7 for two human donor samples.

As shown in FIG. 7, left panel, T cell proliferation was inhibited by addition of ATP. T cell proliferation was restored in the presence of the anti-CD73 antibodies (middle and right hand panels). All of the 2H4+2Lx humanized variants were able to restore T cell proliferation with comparable or better efficacy comparable to or better than the parental 2HP2LP antibody. All 2H4+2Lx variants were more potent than the parental 2HP2LP to restore T cell proliferation in these settings. These results were in accordance with those obtained in T cell proliferation assay using AMP as inhibitor.

In summary, the substitutions introduced in the H4+ and 2H4+ variable regions together with the substitutions introduced into the L1 and 2L1 chains (and the L2, L3, L4, 2L2, 2L3, 2L4 chains) appear to restore (and even improve on) the important functional properties of their parental murine antibodies, yet have human framework regions and therefore lower risk of immunogenicity in humans. The 2H4+2Lx antibodies are the most potent of all.

### Example 8

### Human Dose Projections of Anti-CD73 Antibodies

A PK/PD model was built to characterize the relationship between serum concentrations of the 2H4+2L1 anti-CD73 antibody (heavy chain of SEQ ID NO: 47 and light chain of SEQ ID NO: 48) and CD73 occupancy in blood. First, a PK model was built to describe the anti-CD73 antibody serum concentrations, using the data from the non-GLP and GLP toxicology studies in cynomolgus monkeys.

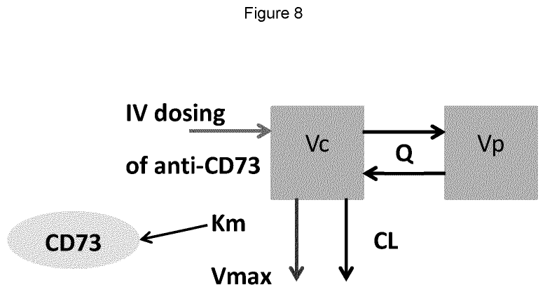

Pharmacokinetics of therapeutic mAbs were modelled using a two-compartment model (Deng et al. 2011 MAbs 3(1): 61-66). Based on preclinical PK results in cynomolgus monkeys, anti-CD73 antibody was expected to display PK properties similar to other therapeutic mAbs in humans, except for compound-specific target-mediated drug disposition (TMDD). This TMDD effect can was modelled by an additional non-linear elimination added to the model (Wang et al. 2016 Biopharm. Drug Dispos. 37: 51-65). A two-compartment model with parallel first order (linear) and saturable (non-linear, Michaelis-Menten) elimination from the central compartment was developed to adequately describe the observed PK of anti-CD73 antibody following repeated IV administration in cynomolgus monkeys, as illustrated in FIG. 8.

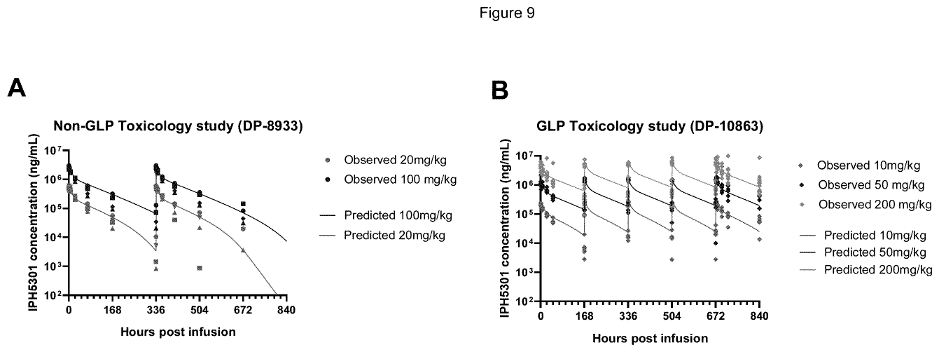

The overlay of predicted and observed anti-CD73 antibody concentrations in non-GLP and GLP toxicology studies is shown in FIG. 9. Panel A represents a model fitting to observed PK data from non-GLP toxicology study; Panel B shows the model fitting to observed PK data from GLP toxicology study; Symbols represent the observed data; solid lines represent the model prediction.

The PK model developed for cynomolgus monkeys was used for human PK prediction. In two anti-CD73 antibody toxicology studies performed in cynomolgus monkeys, linear PK parameters for anti-CD73 antibody were found close to those found in NHP studies described in the literature (Deng et al., 2011), except a higher clearance (CL). Hence, IgG1 PK parameters in human were used for the prediction of the linear component of the human PK, except for both the Clearance and the compound-specific TMDD.

CD73 saturation levels for the PD model are projected from the Km, representing EC₅₀, the anti-CD73 antibody serum concentration leading to 50% receptor occupancy.

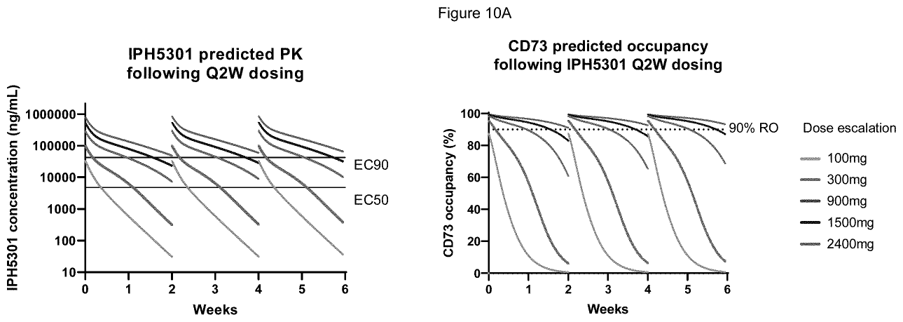

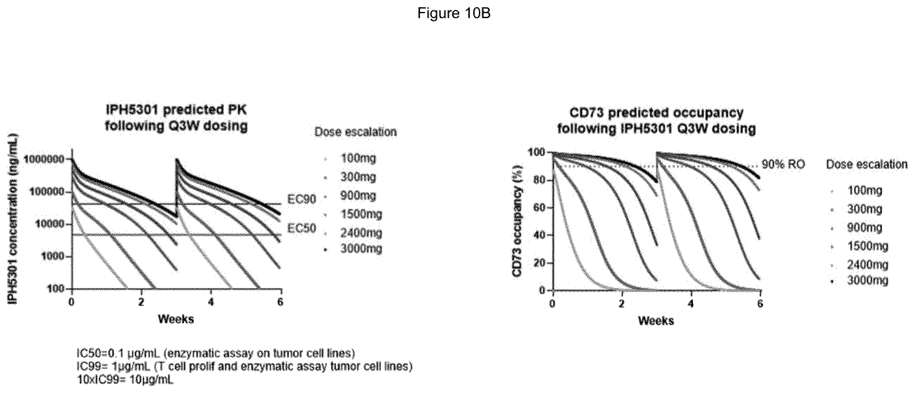

The predicted human PK and CD73 receptor occupancy profiles over the proposed clinical dose for administration every two weeks are shown in FIG. 10A (lines correspond, from bottom to top, to increasing doses). The predicted human PK and CD73 receptor occupancy profiles over different doses for administration every three weeks are shown in FIG. 10B (lines correspond, from bottom to top, to increasing doses).

The starting dose of 100 mg is predicted to have a Cmax of 32.5 μg/mL, which is below the EC90 (target saturation at 90%) of 42.3 μg/mL, estimated by the Michaelis Menten constant Km from the PK model. At this starting dose of 100 mg, approximately 90% of CD73 occupancy is predicted at Cmax, with a return to 0 before the next dose.

Moreover, at this starting dose of 100 mg (2-weekly), anti-CD73 antibody concentration is predicted to return to lower than 50 ng/ml before the next dose. This concentration mediates lower than 100% enzymatic inhibition, in in vitro assays on human serum (IC50=12.3ng/ml), on A375 tumor cells (IC50=34 ng/ml), on MDA-MB231 tumor cells (IC50=126.8 ng/ml) and on reversion of AMP inhibited T cell proliferation on CD4+ and CD8+ T cells (IC50=8.1 ng/ml and 7.7 ng/ml respectively). This concentration is also similar to, or lower than, EC50 values of anti-CD73 antibody binding to CD73+ tumor cells, and to anti-CD73 antibody affinity for recombinant soluble human CD73. This further suggests that the starting dose will result in loss of full saturation of CD73 before the next dose.

The three first escalating dose of 100, 300 and 900 mg when administered every two weeks are predicted to result in CD73 occupancy lower than 80% before the next dose, with anti-CD73 antibody concentration below the EC90 before the next dose. The two highest dose levels of 1500 mg and 2400 mg when administered every two weeks are predicted to maintain CD73 occupancy above 90% throughout the treatment period.

### Example 9

### A phase 1 First-in-human Study of anti-CD73 in Combination With Chemotherapy With Trastuzumab in Patients With Advanced Solid Tumors

2H4+2L1 anti-CD73 antibody (IPH5301) is a humanized IgG1 antagonist monoclonal antibody, with a functionally silent Fc domain. It specifically binds to CD73 and is designed to enhance anti-tumor immune responses by inhibiting the enzymatic activity of CD73 expressed on cells in the tumor microenvironment. Preclinical models have shown differentiated and superior in vitro activity compared to other agents targeting CD73 that are currently in clinical testing. This is the first-in-human clinical trial investigating IPH5301.

The primary objective is to evaluate the safety profile and the maximum tolerated dose (MTD)/recommended phase 2 dose (RP2D) of IPH5301 in combination with chemotherapy with or without trastuzumab in patients with selected advanced solid tumors. The secondary objectives will include evaluation of the preliminary clinical activity of IPH5301 in combination with chemotherapy with or without trastuzumab.

The study will include patients with incurable advanced and/or metastatic cancer, eligible for treatment with paclitaxel and trastuzumab (cohort 2), with no restrictions on number of prior systemic therapies. Patients with HER2-positive breast or gastric carcinoma will be included. Eligibility is based on HER2+ overexpression (+3 by immunohistochemistry or positive by FISH) as determined locally. Prior treatment with paclitaxel, carboplatin and/or trastuzumab in the advanced setting is allowed, unless patient had experienced disease progression within the first 3 months of previous treatment.

Five escalating dose levels of IPH5301 will be evaluated as follows: (100 mg, 300 mg, 900 mg, 1500 mg and 2400 mg). All patients will receive IPH5301 alone on Day 1 (Week 1). On day 15 (start of week 3), chemotherapy and trastuzumab will be added to IPH5301 as follows:
Paclitaxel (175 mg/m², 3 hours infusion) every 3 weeks, and
Trastuzumab (8 mg/kg body weight loading dose, 90 minutes infusion then 6 mg/kg every 3 weeks, 30 minutes).

IPH5301 will be administered as an intravenous infusion over 1 hour irrespective of the tested dose followed by trastuzumab and then chemotherapy. Chemotherapy (in combination with trastuzumab) will be given for a maximum of 6 cycles. During this period, IPH5301 will be administered every two weeks irrespective of the dose level. Patients who show no evidence of disease progression at the end of 6 cycles can continue to receive IPH5301 and trastuzumab for the HER2-positive cohort, both administered on 3-weekly basis, as long as the patient is deriving clinical benefit.

1-42. (canceled)

43. A method of treating a gastric adenocarcinoma or a gastroesophageal junction adenocarcinoma in an individual, comprising administering to the individual a therapeutically effective amount of each of an antibody that binds a human CD73 protein and neutralizes the 5′-ectonucleotidase activity thereof and an antibody that binds a human HER2 protein.

44. The method of claim 43, wherein the antibody binds a human CD73 protein is used in a combination treatment with a chemotherapy agent.

45. The method of claim 43, wherein the treatment comprises administering: (i) the antibody that binds a human CD73 protein every two weeks or every three weeks, (ii) the antibody that binds a human HER2 protein every week or every 3 weeks.

46. The method of claim 44, wherein the treatment comprises administering: (i) the antibody binds a human CD73 protein every two weeks or every three weeks, (ii) the antibody that binds a human HER2 protein every 3 weeks, and (iii) paclitaxel every three weeks.

47. The method of claim 44, wherein a loading dose of antibody that binds a human HER2 protein antibody is administered one week prior to the first three-weekly administration of antibody that binds a human HER2 protein.

48. A method of treating a HER2-positive cancer in an individual in need thereof, comprising administering to the individual a therapeutically effective amount of each of (i) an antibody that binds a human CD73 protein and neutralizes the 5′-ectonucleotidase activity thereof and (ii) an antibody that binds a human HER2 protein.

49. A method of treating a cancer in an individual, comprising administering to the individual an antibody that binds a human CD73 protein and neutralizes the 5′-ectonucleotidase activity thereof, wherein the antibody is an antibody or antibody fragment comprising a light chain variable region having the amino acid sequence of any one of SEQ ID NOS: 43, 44, 45 or 46 and a heavy chain variable region having the amino acid sequence of SEQ ID NO: 42, wherein the antibody is administered to the individual (a) every 2 weeks at a fixed dose (irrespective of body weight or surface area) of 1500-3000 mg, optionally at a dose of 1500 mg, optionally at a dose of 2400 mg, or (b) every 3 weeks at a fixed dose of 2000-3000 mg, optionally at a dose of 2100 mg, 2200 mg, 2300 mg, 2400 mg, 2500 mg, 2600 mg, 2700 mg, 2800 mg, 2900 mg or 3000 mg.

50. The method of claim 49, wherein the treatment comprises administering to the individual:
(i) an antibody that binds a human CD73 protein, administered (a) every 2 weeks at a dose of 1500-3000 mg or (b) every three weeks at a dose of 2000-3000 mg, and
(ii) an antibody that binds a human HER2 protein, administered every 3 weeks by intravenous administration at a dose of 3-6 mg/kg body weight or by subcutaneous administration at a dose of 600 mg, and optionally
(iii) paclitaxel, administered every 3 weeks at a dose of 175 mg/m² body surface area.

51. The method of claim 49, wherein the treatment comprises administering to the individual:
(i) an antibody that binds a human CD73 protein (a) every 2 weeks at a dose of 1500-3000 mg or (b) every three weeks at a dose of 2000-3000 mg, and
(ii) an antibody that binds a human HER2 protein, administered in a loading dose of 8 mg/kg followed one week later by treatment doses of 6 mg/kg body weight administered every 3 weeks; and, optionally,
(iii) paclitaxel, every 3 weeks at a dose of 175 mg/m² body surface area.

52. The method of claim 51, wherein the first administration of antibody that binds a human HER2 protein and the first administration of paclitaxel occur two weeks after the first dose of antibody that binds a human CD73 protein.

53. The method of claim 49, wherein the treatment comprises administering to the individual:
(i) an antibody that binds a human CD73 protein, (a) every 2 weeks at a dose of 1500 or 2400 mg or (b) every three weeks at a dose of 2000-3000 mg, and
(ii) an antibody that binds a human HER2 protein, every 3 weeks, at a dose of 3-6 mg/kg body weight, and optionally
(iii) paclitaxel, every 3 weeks at a dose of 175 mg/m² body surface area.

54. The method of claim 49, wherein the treatment comprises administering to the individual:
(i) an antibody that binds a human CD73 protein, (a) every 2 weeks at a dose of 1500 or 2400 mg or (b) every three weeks at a dose of 2000-3000 mg, and
(ii) an antibody that binds a human HER2 protein, administered in a loading dose of 8 mg/kg followed one week later by treatment doses of 6 mg/kg body weight administered every 3 weeks; and, optionally,
(iii) paclitaxel, every 3 weeks at a dose of 175 mg/m² body surface area.

55. The method of claim 50, wherein said antibody that binds a human CD73 protein, antibody that binds a human HER2 protein and/or chemotherapy agent are administered intravenously.

56. The method of claim 50, wherein the antibody that binds a human CD73 protein and the antibody that binds a human HER2 protein are formulated for separate administration.

57. The method of claim 49, wherein the cancer is a HER2 positive cancer and is a breast cancer, a gastric adenocarcinoma or a gastroesophageal junction adenocarcinoma.

58. The method of claim 49, wherein the individual has received a prior course of therapy with a chemotherapy agent, or with an antibody that binds a human HER2 protein.

59. The method of claim 49, wherein the antibody that binds a human CD73 protein is an antibody or antibody fragment comprising a light chain variable region having the amino acid sequence of SEQ ID NO: 43 and a heavy chain variable region having the amino acid sequence of SEQ ID NO: 42, or a function-conservative variant thereof.

60. The method of claim 49, wherein the antibody that binds a human HER2 protein is an antibody or antibody fragment comprising a light chain variable region having the amino acid sequence of SEQ ID NO: 24 and a heavy chain variable region having the amino acid sequence of SEQ ID NO: 23.

61. The method of claim 60, wherein the antibody that binds a human HER2 protein comprises a light chain having the amino acid sequence of SEQ ID NO: 26 and a heavy chain having the amino acid sequence of SEQ ID NO: 25.

62. The method of claim 59, wherein the antibody that binds a human CD73 protein comprises a light chain having the amino acid sequence of SEQ ID NO: 48 and a heavy chain having the amino acid sequence of SEQ ID NO: 47.
